(19) United States
(12) Patent Application Publication
(10) Pub. No.: US 2018/0064712 A1
(43) Pub. Date: Mar. 8, 2018

(54) METHODS OF REDUCING IMMUNE CELL ACTIVATION AND USES THEREOF

(71) Applicant: Washington University, St. Louis, MO (US)

(72) Inventors: Erika L. Pearce, St. Louis, MO (US); Michael D. Buck, St. Louis, MO (US); David O'Sullivan, St. Louis, MO (US); Francesc Baixauli, Munchen (DE)

(73) Assignee: Max Planck Institute, Munchen (DE)

(21) Appl. No.: 15/617,911

(22) Filed: Jun. 8, 2017

Related U.S. Application Data
(60) Provisional application No. 62/347,403, filed on Jun. 8, 2016.

(51) Int. Cl. A61K 31/517; A61K 31/15; A61K 45/06

(52) U.S. Cl. CPC A61K 31/517; A61K 45/06; A61K 31/15

(57) ABSTRACT

The present invention encompasses methods of reducing inflammatory immune cell activation and inflammation via inhibiting mitochondrial fission.

### CROSS REFERENCE TO RELATED APPLICATION

This application claims priority to U.S. provisional application No. 62/347,403, filed on Jun. 8, 2016, entitled “Methods Of Reducing Immune Cell Activation And Uses Thereof” which is expressly incorporated by reference herein in its entirety.

### GOVERNMENTAL RIGHTS

This invention was made with government support under R01 CA181125, R01 AI091965 and DGE-1143954 awarded by National Institutes of Health (NIH) and National Science Foundation (NSF). The government has certain rights in the invention.

### FIELD OF THE INVENTION

The present invention encompasses methods of reducing inflammation.

### BACKGROUND OF THE INVENTION

Inflammation is the body's protective response to injury and infection. It is a complex process involving many cell types, as well as different components of blood. The inflammatory process works quickly to destroy and eliminate foreign and damaged cells, and to isolate the infected or injured tissues from the rest of the body. Inflammatory disorders arise when inflammation becomes uncontrolled, and causes destruction of healthy tissue. There are dozens of inflammatory disorders. Many occur when the immune system mistakenly triggers inflammation in the absence of infection, such as inflammation of the joints in rheumatoid arthritis. Others result from a response to tissue injury or trauma but affect the entire body. The etiology of diseases and syndromes such as rheumatoid arthritis, inflammatory bowel diseases, sepsis, obesity, type II diabetes, atherosclerosis, and multiple sclerosis have been attributed to inflammation induced by aberrant immune cell responses to endogenous and exogenous stimuli. Current treatments that have found varying levels of success target the effector molecules produced from immune cells that mediate inflammatory response, such as IL-1 beta. However, there is a need in the art for new, effective methods of treating inflammation to alleviate many common diseases.

### SUMMARY OF THE INVENTION

One aspect the disclosure encompasses a method of reducing immune cell activation in a subject by administering a composition that includes compounds that inhibit mitochondrial fission, compounds that promote mitochondrial fusion, or a combination of mitochondrial fission inhibitors and mitochondrial fusion promoters to the subject. The compounds that inhibit mitochondrial fusion may be inhibitors of Drp1, such as Mdivi-1. A compound that promotes mitochondrial fusion may be M1. The immune cells activated may be T cells, macrophages, or dendritic cells.

Another aspect of the disclosure encompasses a method of reducing inflammation in a subject by administering a composition that may include compounds that inhibit mitochondrial fission, compounds that promote mitochondrial fusion, or a combination of mitochondrial fission inhibitors and mitochondrial fusion promoters to the subject. The compounds that inhibit mitochondrial fusion may be inhibitors of Drp1, such as Mdivi-1. A compound that promotes mitochondrial fusion may be M1. The inflammation may be due to induction of aerobic glycolysis, sepsis, obesity, rheumatoid arthritis, multiple sclerosis, Crohn's disease, irritable bowel disease, colitis, psoriasis, inflammatory liver disease, or nonalcoholic fatty liver disease. Administering the drug may also decrease the number of inflammatory T cell subset, such a Th17 cells and decrease the number of T regulatory cells.

Another aspect of the disclosure encompasses a method of reducing detectable markers of infection by administering a composition that may include compounds inhibit mitochondrial fission, compounds that promote mitochondrial fusion, or a combination of mitochondrial fission inhibitors and mitochondrial fusion promoters to the subject. The markers of inflammation that are reduced may be cytokines, immune cells, lactate, C-reactive protein, mitochondrial structure, mitochondrial function, and metabolic function of immune cells. The markers of inflammation may be detected in a biological sample of the subject. The compounds that inhibit mitochondrial fusion may be inhibitors of Drp1, such as Mdivi-1.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, FIG. 1C, FIG. 1D, and FIG. 1E depict images and immunoblots showing that effector and memory T cells possess distinct mitochondrial morphologies. (FIG. 1A) C57BL/6 mice were infected i.p. with 1×10⁷ CFU LmOVA. Effector (T_{E}, CD44^{hi}CD62L^{lo}, 7 days post infection) and memory T (T_{M}, CD44^{hi} CD62L^{hi}, 21 days post infection) cells were sorted and analyzed by EM as well as (FIG. 1B) IL-2 T_{E} and IL-15 T_{M} cells generated from differential culture of OT-I cells activated with OVA peptide and IL-2 using IL-2 or IL-15, scale bar=0.5 μm. (FIG. 1C, FIG. 1D) Mitochondrial morphology was analyzed in live OT-I PhAM cells over time before and after αCD3/CD28 activation and differential cytokine culture by spinning disk confocal microscopy. Mitochondria are green (GFP) and nuclei are blue (Hoechst). (FIG. 1C) Scale bar=5 μm, (FIG. 1D) Scale bar=1 μm. (FIG. 1E) Immunoblot analysis of cell protein extracts from (FIG. 1C), probed for Mfn2, Opa1, Drp1, phosphorylated Drp1 at Ser616 (Drp1p^{S616}), and β-actin. Results representative of 2 experiments. See also FIG. 7.

FIG. 2A, FIG. 2B, FIG. 2C, FIG. 2D, FIG. 2E, FIG. 2F, FIG. 2G, and FIG. 2H depict graphs, images and flow cytometry plots showing that memory T cell development and survival, unlike effectors, requires mitochondrial fusion. (FIG. 2A and FIG. 2B) Relative in vitro survival ratios of Mfn1, Mfn2, or Opa1 deficient (CD4 Cre⁺, −/−) to wild-type control (CD4 Cre⁻, +/+) OT-I IL-2 T_{E} and IL-15 T_{M} cells (*p=0.0465). Data normalized from 2-3 independent experiments shown as mean±SEM. (FIG. 2C) Mitochondrial morphology of OT-I Opa1 wild-type and Opa1 knockout IL-2 T_{E} and IL-15 T_{M} cells analyzed by EM (scale bar=0.5 μm, one experiment represented) and (FIGS. 2D and 2F) Seahorse EFA. (FIG. 2D) bar graph represents ratios of O₂ consumption rates (OCR, an indicator of OXPHOS) to extracellular acidification rates (ECAR, an indicator of aerobic glycolysis) at baseline and (FIG. 2E) spare respiratory capacity (SRC) (% max OCR after FCCP injection of baseline OCR) of indicated cells (*p<0.03, **p=0.0079). Data from 3 experiments shown as mean±SEM. (FIG. 2F, FIG. 2G, and FIG. 2H) 10⁴ OT-I Opa1^{+/+} or Opa1^{-/-} T cells were transferred i.v. into C57BL/6 CD90.1 mice infected i.v. with 1×10⁷ CFU LmOVA. Blood was analyzed by flow cytometry at indicated time points post infection. After 21 days, mice were challenged i.v. with 5×10⁷ CFU LmOVA and blood analyzed post challenge (p.c.). (FIG. 2F) % Donor K^{b}/OVA⁺ and CD90.2⁺ live cells shown in representative flow plots and (FIG. 2G) line graph with mean±SEM. (*p=0.0238, **p<0.005). (FIG. 2H) Number of donor K^{b}/OVA⁺ cells isolated from spleens of infected mice shown as mean±SEM (*p=0.0126). (FIG. 2F, FIG. 2G, FIG. 2H) Results representative of 2 experiments (n=9-11 per group). See also FIG. 8.

FIG. 3A, FIG. 3B, FIG. 3C, FIG. 3D, FIG. 3E, FIG. 3F, FIG. 3G, FIG. 3H, FIG. 3I, FIG. 3J, FIG. 3K, FIG. 3L, FIG. 3M, FIG. 3N, and FIG. 3O depict a schematic, graphs and images showing that enhancing mitochondrial fusion promotes the generation of memory-like T cells. (FIG. 3A, FIG. 3B, FIG. 3C, FIG. 3D, FIG. 3E, FIG. 3F, FIG. 3I, FIG. 3J, FIG. 3K, FIG. 3L, FIG. 3M, FIG. 3N, and FIG. 3O) OVA peptide and IL-2 activated OT-I cells were differentiated into IL-2 T_{E} or IL-15 T_{M} cells for 3 days in the presence of DMSO control or fusion promoter M1 and fission inhibitor Mdivi-1 (M1+Mdivi-1) as shown in (FIG. 3A) pictorially. (FIG. 3B) Representative spinning disk confocal images from 3 experiments of live cells generated from OT-I PhAM mice. Mitochondria are green (GFP) and nuclei are blue (Hoechst), scale bar=5 μm. (FIGS. 3C, and 3D) Cells stained with MitoTracker Green and analyzed by flow cytometry. Relative MFI (left) from 6 experiments shown as mean±SEM (*p=0.0394, **p=0.0019) with representative histograms (right). (FIG. 3D and FIG. 3F) Baseline OCR and SRC of indicated cells from 3-4 experiments shown as mean±SEM (*p=0.0485, ***p<0.0001). (FIG. 3G and FIG. 3H) CD62L expression analyzed by flow cytometry of indicated cells. Relative MFI (left) from 7 experiments shown as mean±SEM (*p=0.0325, **p=0.0019, ***p<0.0001) with representative histograms (right). (FIG. 3I) OCR of indicated cells at baseline and in response to PMA and ionomycin stimulation (PMA+iono), oligomycin (Oligo), FCCP, and rotenone plus antimycin A (R+A). Data represents 2 experiments shown as mean±SEM. (FIG. 3J and FIG. 3K) OT-I cells were transduced with either empty (Control), Mfn1, Mfn2, or Opa1 expression vectors, sorted, and cultured to generate IL-2 T_{E} cells. (FIG. 3J) Histograms representative of 4 experiments of cells stained for MitoTracker Deep Red and (FIG. 3K) OCR data at baseline of transduced cells from 2 experiments. (FIG. 3L, FIG. 3M, FIG. 3N, FIG. 3O) 1-2×10⁶ IL-2 T_{E} cells cultured with DMSO (gray diamonds) or M1+Mdivi-1 (blue squares) were transferred into congenic C57BL/6 recipient mice. Cell counts of donor cells recovered 2 days later from the (FIG. 3L) spleen (***p=0.005) and (FIG. 3M) peripheral lymph nodes (pLNs, ***p=0.0006). (FIG. 3N) Blood from recipient mice analyzed for % donor K^{b}/OVA⁺ cells post transfer and challenge with 1×10⁷ CFU LmOVA by flow cytometry (*p=0.0150, n=5 per group). (FIG. 3O) Donor K^{b}/OVA⁺ cells recovered from recipient spleens 6 days post challenge (*p=0.0383). (FIG. 3L, FIG. 3M, FIG. 3N, FIG. 3O) Data represents 2 experiments shown as mean±SEM. See also FIG. 9.

FIG. 4A, FIG. 4B, FIG. 4C, FIG. 4D, FIG. 4E, and FIG. 4F depict graphs and images showing that mitochondrial fusion improves adoptive cellular immunotherapy against tumors. (FIG. 4A, FIG. 4B) C57BL/6 mice were inoculated s.c. with 1×10⁶ EL4-OVA cells. (FIG. 4A) After 5 or (FIG. 4B) 12 days, 1×10⁶ or 5×10⁶ OT-I IL-2 T_{E} cells cultured with DMSO or M1+Mdivi-1 were transferred i.v. into recipient mice and tumor growth assessed. Data represents 2 experiments shown as mean±SEM (n=5 per group, *p<0.05, **p<0.005). (FIG. 4C, FIG. 4D, FIG. 4E, FIG. 4F) Human CD8⁺ PBMCs were activated with αCD3/CD28+IL-2 to generate IL-2 T_{E} cells. (FIG. 4C) Confocal images of indicated treated cells where mitochondria are green (MitoTracker) and nuclei are blue (Hoechst). Representative images from 2 of 4 biological donors, scale bar=5 μm. (FIG. 4D, FIG. 4E) OCR/ECAR ratios and SRC of indicated cells from 2 separate donors shown as mean±SEM (*p=0.0303, **p<0.005, ***p<0.0001). (FIG. 4F) MitoTracker Green staining and CD62L, CD45RO, and CCR7 expression analyzed by flow cytometry shown with representative histograms from 4-6 biological replicates. See also FIG. 10.

FIG. 5A, FIG. 5B, FIG. 5C, FIG. 5D, and FIG. 5E depict graphs showing that fusion promotes memory T cell metabolism, but Opa1 is not required for FAO. OCR measured at baseline and in response to media, etomoxir (Eto) and other drugs as indicated of (FIG. 5A) IL-2 T_{E} cells cultured in DMSO or M1+Mdivi-1, (FIG. 5B) control or Opa1 transduced IL-2 T_{E} cells, (FIG. 5C) Opa1^{+/+} and Opa1^{-/-} IL-2 T_{E} cells cultured in DMSO or M1+Mdivi-1 (FIG. 5D) or without drugs, and (FIG. 5E) ex vivo donor OT-I Opa1^{+/+} and Opa1^{-/-} day 7 T_{E} cells derived from LmOVA infection. Data representative of 2 independent experiments shown as mean±SEM (***p<0.0001). See also FIG. 11.

FIG. 6A, FIG. 6B, FIG. 6C, FIG. 6D, FIG. 6E, FIG. 6F, FIG. 6G, FIG. 6H, FIG. 6I, FIG. 6J, FIG. 6K, FIG. 6L, FIG. 6M, and FIG. 6N depict graphs and images showing that mitochondrial cristae remodeling signals metabolic pathway engagement. (FIG. 6A) Basal ECAR of OT-I Opa1^{+/+} and Opa1^{-/-} IL-2 T_{E} cells (left) and day 7 T_{E} cells isolated ex vivo after adoptive transfer from LmOVA infection (right). Data combined from 2-3 experiments (*p=0.0412, ***p<0.0001). (FIG. 6B) OCR at baseline and after indicated drugs, representative of 2 experiments shown as mean±SEM, and (FIG. 6C) D-Glucose-¹³C1,2 trace analysis of OT-I Opa1^{+/+} and Opa1^{-/-} IL-2 T_{E} cells. Each lane represents separate mice with a technical replicate. (FIG. 6D) EM analysis of mitochondrial cristae from T_{E} and T_{M} cells isolated after LmOVA infection and (FIG. 6E) in vitro cultured IL-2 T_{E} and T_{M} cells. Data representative of 2 experiments, scale bar=0.25 μm. Relative proton leak (ΔOCR after oligomycin and subsequent injection of rotenone plus antimycin A) of (FIG. 6F) Opa1^{+/+} and Opa1^{-/-} IL-2 T_{E}, (FIG. 6G) infection elicited T_{E} and T_{M}, and (FIG. 6H) IL-2 T_{E} and IL-15 T_{M} cells. (FIG. 6F, FIG. 6G, FIG. 6H) Data combined from 2-4 experiments shown as mean±SEM (p**<0.005, ***p<0.0001). (FIG. 6I) EM analysis of IL-15 T_{M} cell-mitochondrial cristae before and after αCD3/CD28-conjugated bead stimulation over hours, scale bar=0.2 μm and represents one experiment. (FIG. 6J) Immunoblot analysis of ER protein Calnexin and ETC complexes (CI-NDUFB8, CII-SDHB, CIII-UQCRC2, CIV-MTC01, CV-ATP5A). Equivalent numbers of IL-2 T_{E} and IL-15 T_{M} cells were lysed in native lysis buffer followed by digitonin solubilization of intracellular membranes. Pellet (P) and solubilized supernatant (S) fractions were resolved on a denaturing gel. Data representative of 2 experiments. (FIG. 6K) IL-15 T_{M} cell, (FIG. 6L, FIG. 6M) bone marrow-derived dendritic cell (BM-DCs) and macrophage (BM-Macs) % ECAR measured at baseline and after media, αCD3/CD28-conjugated bead, LPS, or LPS+IFN-γ injection as indicated. Data are baselined prior to or right after injection with stimuli. (FIG. 6N) BM-Macs stained for intracellular Nos2 protein by flow cytometry with MFI values (left) and representative histogram (right). (FIG. 6K, FIG. 6L, FIG. 6M, FIG. 6N) Data shown as mean±SEM and represent 2-3 experiments (***p<0.0001). See also FIG. 12.

FIG. 7 depicts a schematic showing the in vitro differentiation of IL-2 T_{E} and IL-15 T_{M} cells approximate T cell response conditions that generate T_{E} and T_{M} cells in vivo (Related to FIG. 1). OT-I cells were activated with IL-2 and either OVA peptide or αCD3/CD28 for 3 days and then differentially cultured in IL-2 or IL-15 for an additional 3 days to generate IL-2 T_{E} and IL-15 T_{M} cells, respectively.

FIG. 8A, FIG. 8B, FIG. 8C, FIG. 8D, FIG. 8E, FIG. 8F and FIG. 8G depict graphs, immunoblots and flow cytometry plots showing the assessment of genetic deletion of mitochondrial fusion proteins in IL-2 T_{E} and IL-15 T_{M} cells and of donor T_{E} cells generated from infection. (Related to FIG. 2). (FIG. 8A, FIG. 8B, FIG. 8C) IL-2 T_{E} and IL-15 T_{M} cells were cultured from (FIG. 8A) OT-I Mfn1 floxed, (FIG. 8B) OT-I Mfn2 floxed, (FIG. 8C) OT-I Opa1 floxed mice crossed to CD4 Cre transgenic mice to generate T cells conditionally deleted for proteins that mediate mitochondrial fusion (+/+ are CD4 Cre⁻ and −/− are CD4 Cre+). Efficiency of deletion by cre recombinase analyzed by (FIG. 8A) qPCR and (FIG. 8B, FIG. 8C) immunoblot. (FIG. 8D, FIG. 8E, FIG. 8F) Flow cytometry analysis of short-lived effector cells (SLEC, KLRG1^{hi} CD127^{lo}) and memory precursor effector cells (MPEC, KLRG1^{lo} CD127^{hi}) generated at day 7 post infection from OT-I Opa1^{+/+} and OT-I Opa1^{-/-} cells transferred into congenic recipients infected with LmOVA. Representative flow dot plots and scatter dot plots with mean±SEM bars. Each dot represents individual mice (n=8-9 per genotype), ***p<0.0001. (FIG. 8G) OCR analysis of day 10 post-infection OT-I Opa1^{+/+} and Opa1 donor cells at baseline and after oligomycin (Oligo), FCCP, and rotenone plus antimycin A (R+A) injections. Data is representative of 2 experiments shown as mean±SEM.

FIG. 9A, FIG. 9B, FIG. 9C, FIG. 9D, FIG. 9E, FIG. 9F, FIG. 9G, and FIG. 9H, FIG. 9I, FIG. 9J, FIG. 9J, FIG. 9K, FIG. 9L, and FIG. 9M depict graphs showing the assessment of T cell phenotype and metabolism following pharmacological or enhancement of mitochondrial fusion (Related to FIG. 3). (FIG. 9A, FIG. 9B, FIG. 9C, FIG. 9D, FIG. 9E, FIG. 9F) IL-2 T_{E} and IL-15 T_{M} cells generated from OT-I mice were treated with DMSO control or M1+Mdivi-1. (FIG. 9A) ECAR of indicated cells at baseline and after PMA and ionomycin (PMA+iono) stimulation, oligomycin (Oligo), FCCP, and rotenone plus antimycin A (R+A). (FIG. 9B) qPCR analysis of relative mitochondrial DNA (mtDNA) to nuclear DNA (nDNA) ratios of indicated cells. (FIG. 9C, FIG. 9D) ECAR (left) and OCR/ECAR ratios (right) of indicated cells under basal conditions. (FIG. 9E) Histograms of membrane potential (CMxROS, TMRM) and mitochondrial ROS (MitoSOX) using indicated fluorescent dyes and (FIG. 9F) KLRG1, CD127, CCR7, and CD25 surface marker expression of indicated cells analyzed by flow cytometry. (FIG. 9G, FIG. 9H, FIG. 9I, FIG. 9J, FIG. 9J, FIG. 9K, FIG. 9L, and FIG. 9M) OT-I IL-2 T_{E} cells were activated and transduced with empty vector (Control), Mfn1, Mfn2, or Opa1 expressing retrovirus. (FIG. 9G, FIG. 9H, FIG. 9I) ECAR, OCR/ECAR, and SRC analyzed by Seahorse EFA, (FIG. 9J) KLRG1, CD127, CCR7, CD25 and PD-1 surface marker expression assessed by flow cytometry, and (FIG. 9K, FIG. 9L, and FIG. 9M) gene expression analysis by qPCR. (FIG. 9A, FIG. 9B, FIG. 9C, FIG. 9D, FIG. 9E, FIG. 9F, FIG. 9G, FIG. 9H, FIG. 9I, FIG. 9J, FIG. 9J) Data are shown as mean±SEM and are representative or (FIG. 9B, FIG. 9C, FIG. 9D, FIG. 9G, FIG. 9H, FIG. 9I) combined from 2-3 experiments, not significant (ns), “p<0.001, ***p<0.0001.

FIG. 10A, FIG. 10B, FIG. 10C, FIG. 10D, FIG. 10E and FIG. 10F depict graphs and flow cytometry plots showing the examination of mouse and human IL-2 T_{E} cells after enforcing mitochondrial fusion with drugs (Related to FIG. 4). (FIG. 10A, FIG. 10B, FIG. 10C) Flow cytometry analyses of IL-2 T_{E} cells previously cultured with DMSO or M1+Mdivi-1 combined from 3 biological replicates. Cells were not subjected to further treatment with DMSO or M1+Mdivi-1 during experiment assays. (FIG. 10A) Cytolysis of EL4-OVA target cells at indicated concentrations. (FIG. 10B) Proliferation after restimulation with αCD3/CD28. (FIG. 10C, FIG. 10D, FIG. 10E) Intracellular cytokine staining after 4 hours stimulation with PMA and ionomycin. Relative MFI (left) with mean±SEM and representative contour plots (right) with percentage of cytokine positive cells indicated in gated cells and MFI in bold, *p<0.05. Gates based on unstimulated cells (not shown). (FIG. 10F) Human CD8+ PBMCs were activated with αCD3/CD28+IL-2 to generate IL-2 T_{E} cells and subjected to DMSO or M1+Mdivi-1 treatment. KLRG1, CD127, CD45RA, and CD25 surface marker expression analyzed by flow cytometry shown with representative histograms from 4-6 biological replicates.

FIG. 11A, FIG. 11B, and FIG. 11C depict graphs showing bioenergetics analysis after promoting mitochondrial fusion in T cells and macrophages (Related to FIG. 5). (FIG. 11A) OCR of IL-15 T_{M} DMSO or M1+Mdivi-1 treated cells measured at baseline and in response to media, etomoxir (Eto), oligomycin (Oligo), FCCP, and rotenone plus antimycin A (R+A). (FIG. 11B) Bone marrow derived macrophages were cultured overnight with IL-4 (M2) or without (M0) and either DMSO or M1+Mdivi-1 overnight. OCR analyzed at baseline and after injection of mitochondrial inhibitors as indicated. (FIG. 11C) Baseline ECAR relative to DMSO controls of IL-2 T_{E} Opa1^{+/+} and Opa1^{-/-} cells cultured in DMSO or M1+Mdivi-1. Data are shown as mean±SEM and are representative of 2-3 experiments.

FIG. 12A, FIG. 12B, FIG. 12C, FIG. 12D, FIG. 12E, FIG. 12F, FIG. 12G, and FIG. 12H depict images, graphs and immunoblots showing T cell activation induces cristae remodeling that regulates metabolism (Related to FIG. 6). (FIG. 12A) Activated Opa1^{+/+} and Opa1^{-/-} IL-2 T_{E} cells were cultured overnight with D-Glucose-¹³C₁₂ and traced for incorporation by mass spectrometry. Heat map representation of % labeled carbons in listed metabolites. (FIG. 12B, FIG. 12C, FIG. 12D, FIG. 12E) Spleens from either polyclonal wild-type (+/+) or Opa1 deficient T cell animals (−/− Opa1 T) were isolated and surface marker expression assessed (CD44, CD62L on CD3⁺ CD8⁺ gates) by flow cytometry (top) or were further purified for CD8 T cells to assess OCR and ECAR at baseline (below) by Seahorse EFA. Data presented with mean±SEM from n=6 per genotype, ***p<0.0001. (FIG. 12F) EM images of IL-15 T_{M} cell mitochondria over time before and after PMA and ionomycin stimulation from one experiment. Scale bar=0.5 pm. (FIG. 12G, FIG. 12H) Immunoblot analysis of ETC complexes (CI-NDUFB8, CII-SDHB, CIII-UQCRC2, CIV-MTC01, CV-ATP5A) and OMM protein Tom20. Equivalent numbers of IL-2 T_{E} and IL-15 T_{M} cells lysed in native lysis buffer followed by digitonin solubilization of intracellular membranes. Pellet (P) and solubilized supernatant (S) fractions were resolved on a denaturing gel. Second experiment represented from FIG. 6J.

FIG. 13A, FIG. 13B, FIG. 13C, FIG. 13D, FIG. 13E, FIG. 13F, FIG. 13G, FIG. 13H, FIG. 13I, FIG. 13J, FIG. 13K, FIG. 13L depict graphs showing analysis of naïve mice lacking Opa1 in polyclonal T cells compared to wild-type. (FIG. 13A, FIG. 13B, FIG. 13C, FIG. 13D) Percentages of CD3⁺, CD3⁺CD4⁺ (CD4 T), CD3⁺CD8⁺ (CD8 T) cells in the blood from Opa1^{+/+} and Opa1^{-/-} T cell animals (left) and −/− to +/+ ratio of CD4 and CD8 T cells (right). (FIG. 13E) Percentage of CD44^{hi}CD62L^{hi} cells in the blood of indicated animals. (FIG. 13F, FIG. 13G, FIG. 13H) Cell counts of indicated cell populations from the spleen. (FIG. 13I, FIG. 13J, FIG. 13K, FIG. 13L) Histograms (left) and MFI values (right) of surface markers expressed on CD8 T cells from the spleen of Opa1^{+/+} (black) and Opa1^{-/-} (light gray) T cell animals. Data analyzed by flow cytometry and shown with mean±SEM, *p<0.05, **p<0.01, ***p<0.001, ****p<0.0001 (unpaired t test). Dots represent individual mice.

FIG. 14 depicts a graph showing analysis of T cell response from mice lacking Opa1 in polyclonal T cells compared to wild-type after infection. Opa1^{+/+} and Opa1^{-/-} cell mice were infected with 1×10⁷ CFU of LmOVA ΔactA i.p. and serially bled over time to analyze the percentage of K^{b}/OVA bound cells by flow cytometry (n=4-5 per genotype). Mice were rechallenged with 5×10⁷ CFU LmOVA ΔactA i.p. 3 weeks after priming. Data shown as mean±SEM.

FIG. 15 depicts a graph showing analysis of FAO in naïve polyclonal Opa1 wild-type and knockout CD8 T cells. Seahorse analysis of CD8 T cells isolated from Opa1^{+/+} and Opa1 T cell animals. Data shown as mean±SEM combined from 6 biological replicates per genotype.

FIG. 16A, FIG. 16B, and FIG. 16C depict graphs showing polyclonal CD8 T_{E} cell survival and expansion in glucose versus galactose. Polyclonal CD8 T cells were isolated from Opa1^{+/+} and Opa1^{-/-} T cell animals and activated with αCD3/CD28+IL-2 for 3 days in TCM prepared with dialyzed FBS and 11 mM glucose (Glc). After 3 days, the cells were kept in identical media conditions or switched into TCM+IL-2+dialyzed FBS with 11 mM galactose (Gal). (FIG. 16A) Survival was assessed using 7AAD exclusion by flow cytometry. (FIG. 16B, FIG. 16C) Cell number expansion was assessed by acquiring cells that were previously plated at equal concentrations at identical times and speed. Data shown as mean±SEM.

FIG. 17A and FIG. 17B depicts graphs showing the effects of differential concentrations of IL-2 and IL-15 on T cell metabolism. OT-I cells were activated with OVA peptide+IL-2 (100 U/mL) for 3 days and then differentially cultured in IL-2 (100 U/mL) to make IL-2 T_{E} cells, IL-15 (10 ng/mL) to make IL-15 T_{M} cells, or in 10 U/mL IL-2 or 100 ng/mL (IL-15) for 3 days. OCR (FIG. 17A) and ECAR (FIG. 17B) plots from Seahorse analysis of indicated cultured cells at baseline and after oligomycin (Oligo), FCCP, and rotenone plus antimycin A (R+A). Data shown as mean±SEM.

FIG. 18A, FIG. 18B, FIG. 18C, and FIG. 18D depicts images showing the effects of IL-2 withdrawal on mitochondrial morphology in T cells. OT-I PhAM cells were activated with αCD3/28+IL-2 for 3 days (FIG. 18A and FIG. 18B) and then withdrawn from IL-2 for 24 hours (FIG. 18C, and FIG. 18D). Green is GFP (mitochondria) and blue is Hoechst nuclear staining. Scale bar=5 μM.

FIG. 19A and FIG. 19B depict immunoblots showing membrane protein solubility and Drp1 activation of IL-15 T_{M} cells post stimulation. IL-15 T_{M} cells were restimulated with PMA and ionomycin (PMA/iono) and assessed for (FIG. 19A) calnexin and ETC complex proteins membrane solubilization with 1% digitonin (pellet, P and supernatant, S) and (FIG. 19B) Drp1, phosphorylated Drp1 at Ser616 (Drp1^{pS616}) and loading control Tubulin protein expression over time by immunoblot.

FIG. 20A and FIG. 20B depicts graphs showing analysis of protective immunity in mice that received previously primed T cells. 10⁴ OT-I Opa1^{+/+} or Opa1^{-/-} T cells were adoptively transferred i.v. into congenic C57BL/6 recipients and primed i.v. with 10⁷ CFU LmOVA ΔactA. After one week, donor cells were isolated and 10⁶ cells of the previously primed donor cells were transferred i.v. into new congenic C57BL/6 recipients. After donor cells were allowed to contract for one week, the new recipient mice were challenged i.v. with 10⁶ CFU LmOVA and assessed 3 days later for infectious bacterial burden in the spleen (FIG. 20A) and liver (FIG. 20B). Each dot represents individual mice with mean±SEM (*p=0.03, **p=0.0007).

FIG. 21A, FIG. 21B, FIG. 21C, FIG. 21D, FIG. 21E, FIG. 21F, FIG. 21G, FIG. 21H, and FIG. 21I depict the analysis of the regulation of T cell differentiation by mitochondrial dynamics. Naive CD4 T cells were isolated and differentiated in vitro towards Th1, Th2, Th17 and regulatory T cells in the presence of absence of the profusion drugs M1 and Mdivi1. At day 6, cells were reestimulated with PMA/Ion and the level of cytokine production analyzed. Dot plots on the left show the expression of cytokines of vehicle (DMSO) or Mdivi1+M1 treated CD4 T cells analyzed by flow cytometry in Th1 (FIG. 21A, FIG. 21B, FIG. 21C), Th17 (FIG. 21D, FIG. 21E, FIG. 21F), Treg (FIG. 21G, FIG. 21H, and FIG. 21I) culture conditions. Right graphs show quantification of the expression of cytokines in control and Mdivi+M1 treated CD4 T cells in the different polarizations tested.

FIG. 22A, FIG. 22B, FIG. 22C, FIG. 22D, and FIG. 22E depict grafts showing that Targeting Drp1 reduces IL17 production while increasing Foxp3 expression in naive Drp1^{-/-} CD4⁺ T cells skewed into pro-inflammatory Th17 T cells. Naive CD4 T cells from wild-type, Drp1−/+ and Drp−/− mice were isolated and differentiated in vitro towards Th17. At day 6, cells were reestimulated with PMA/Ion and the level of cytokine production analyzed. Dot plots show the expression of IL-17 and Foxp3 in CD4+ analyzed by flow cytometry (FIG. 22A, FIG. 22B, and FIG. 22C, for wild-type, Drp1-1+ and Drp−/−, respectively). Graphs show quantification of the expression of IL-17 (FIG. 22D) and Foxp3 (FIG. 22E).

### DETAILED DESCRIPTION OF THE INVENTION

The present disclosure provides methods of modulating inflammation, immune cell activation, and immune cell number by compounds that modulate mitochondrial dynamics.

Mitochondrial dynamics, or mitochondrial fusion, fission, and cristae remodeling, can signal and control the engagement of specific metabolic pathways. Specific to this application, exposure of T cells, macrophages, and dendritic cells to the Drp1 inhibitor Mdivi-1 in vitro (dampening fission mediated by Drp1) blocks their shift to aerobic glycolysis and as such prevents activation and ensuing effector functions in these cells. Accordingly, blocking mitochondrial fission (directly or via enhancing fusion with other agents such as M1, a promoter of fusion) may be used to treat a variety of inflammatory conditions where an induction of aerobic glycolysis correlates with disease state and/or progression, and an abrogation of this metabolism correlates with a better disease outcome. As such, provided herein are method of reducing immune cell activation and methods of reducing inflammation in a subject.

Various compositions and methods of the invention are described herein below.

### I. Compositions

In an aspect, the present disclosure provides a composition comprising one or more compounds that induces inner mitochondrial membrane remodeling. In another aspect, the present disclosure provides a composition comprising one or more compounds to inhibit mitochondrial fission.

### (a) Composition Comprising One or More Compounds to Induce Inner Mitochondrial Membrane Remodeling

Mitochondrial fusion and fission are dynamic processes that govern efficient cellular metabolism, as well as mitochondrial biogenesis, repair and death. Mitochondrial fission and fusion processes are both mediated by large guanosine triphosphatases (GTPases) in the dynamin family that are well conserved between yeast, flies, and mammals. Their combined actions divide and fuse the two lipid bilayers that surround mitochondria. Fission is mediated by a cytosolic dynamin family member (Drp1 in worms, flies, and mammals and Dnm1 in yeast). Fusion between mitochondrial outer membranes is mediated by membrane-anchored dynamin family members named mitofusin 1 (Mfn1) and mitofusin 2 (Mfn2) in mammals, whereas fusion between mitochondrial inner membranes is mediated by a single dynamin family member called optic atrophy 1 (Opa1) in mammals or Mgm1 in yeast. In addition to promoting mitochondrial fusion, Opa1 has been shown to regulate apoptosis by controlling cristae remodeling and cytochrome c redistribution. Mitochondrial fission and fusion machineries are regulated by proteolysis and posttranslational modifications. Fusion rescues stress by allowing functional mitochondria to complement dysfunctional mitochondria by diffusion and sharing of components between organelles. Mitochondrial fusion can therefore maximize oxidative capacity in response to toxic stress. Fission and fusion events also regulate metabolism, longevity and cell fitness.

In an embodiment, a composition of the disclosure comprises one or more compounds that induce inner mitochondrial membrane remodeling. For example, a compound can alter the localization or movement of mitochondria along cytoskeletal structures. Such an alteration can result in changes in mitochondrial dynamics. Further a compound can alter Kinesins, dynamin motor proteins, or receptor/adaptor proteins (such as Miro 1/2 and Trak1/2). Such proteins are associated with tethering motor proteins to the mitochondria surface and cause alterations in mitochondria localization or structure. In another embodiment, a compound can directly alters interaction of kinesins, dynamin motor proteins or their receptor/adaptor proteins with fission and fusion machinery (e.g. MFNs) which can lead to alterations in mitochondria localization or structure. Still further, a compound that induces inner mitochondrial membrane remodeling can be a compound that affects calcium signaling. Additionally, MFF and/or Fis1 may be targeted to induce inner mitochondrial membrane remodeling.

In an embodiment, a composition of the disclosure comprises one or more compounds to inhibit mitochondrial fission. For example, compounds disclosed in WO 2012158624, the disclosure of which is hereby incorporated by reference in its entirety, may be used. In one embodiment, a compound that inhibits mitochondrial fission may be a compound that selectively inhibits the mitochondrial division dynamin. In another embodiment, a compound that inhibits mitochondrial fission may be a compound that inhibits Drp1 (Dnm1). Non-limiting examples of inhibitors of Drp1 include compounds disclosed in US 2005/0038051, US 2008/0287473 and Cassidy-Stone et al. Developmental Cell 2008; 14(2): 193-204, the disclosures of which are hereby incorporated by reference in their entireties. In still another embodiment, a compound that inhibits mitochondrial fission may be a compound that attenuates Drp1 (Dnm1) self-assembly. In yet another embodiment, a compound that inhibits mitochondrial fission may be a compound that causes the formation of mitochondrial net-like structures. In still another embodiment, a compound that inhibits mitochondrial fission may be a compound that retards apoptosis by inhibiting mitochondrial outer membrane permeabilization. In a different embodiment, a compound that inhibits mitochondrial fission may be a compound that blocks Bid-activated Bax/Bak-dependent cytochrome c release from mitochondria. In certain embodiments, a compound that inhibits mitochondrial fission may be a compound comprising a quinazoline moiety with an unblocked sulfhydryl moiety on the 2-position of the quinazolinone and limited rotation about the 3-position nitrogen-phenyl bond. For example, see compounds A, B, C, D, E, F, G, H, and I as disclosed in Cassidy-Stone et al. Developmental Cell 2008; 14(2): 193-204, which is hereby incorporated by reference in its entirety. Specifically, compound A (Mdivi-1) and compound B are said to have full efficacy relative to Mdivi-1, compounds C, D, and E are said to have moderate efficacy and compounds F, G, H and I are said to have poor efficacy. In a specific embodiment, a compound that inhibits mitochondrial fission is Mdivi-1.

In other embodiments, a compound that inhibits mitochondrial fission does so indirectly. For example, a compound that inhibits mitochondrial fission may be a compound that promotes mitochondrial fusion. Accordingly, in certain embodiments, a composition of the disclosure comprises one or more compounds to promote mitochondrial fusion. In other embodiments, a composition of the disclosure comprises one or more compounds to inhibit mitochondrial fission and further comprises one or more compounds to promote mitochondrial fusion. In one embodiment, a compound that promotes mitochondrial fusion may be a compound that promotes mitochondrial remodeling. In another embodiment, a compound that promotes mitochondrial fusion may be a compound that enhances the activity of one or more of Mfn1, Mfn2 and Opa1 (Mgm1). A compound that enhances the activity of Opa1 may also promote mitochondrial remodeling. In still another embodiment, a compound that promotes mitochondrial fusion may be a compound that induces mitochondrial elongation. In still yet another embodiment, a compound that promotes mitochondrial fusion may be a compound that increases mitochondrial connectivity and integrity. In a different embodiment, a compound that promotes mitochondrial fusion may be a compound that increases ATP5A/B protein levels. In yet another embodiment, a compound that promotes mitochondrial fusion may be a compound that protects cells from MPP+ induced mitochondrial fragmentation and cell death. In certain embodiments, a compound that promotes mitochondrial fusion may be a compound comprising a hydrazone or acylhydrazone moiety. For example, see compounds 1-17 as disclosed in Wang et al. Angew Chem Int Ed 2012; 51: 9302-9305, which is hereby incorporated by reference in its entirety. In a specific embodiment, a compound that promotes mitochondrial fusion may be hydrazone M1.

In certain embodiments, a composition of the disclosure comprises one or more compounds to inhibit mitochondrial fission and/or promote mitochondrial fusion. For example, a composition of the disclosure may comprise 1, 2, 3, 4, 5, 6, 7, 8, 9, or 10 compounds to inhibit mitochondrial fission and/or promote mitochondrial fusion. In a specific embodiment, a composition of the disclosure comprises one compound to inhibit mitochondrial fission. In another embodiment, a composition of the disclosure comprises two compounds to inhibit mitochondrial fission and/or promote mitochondrial fusion. In one embodiment, a first compound may be a compound that inhibits mitochondrial fission and a second compound may be a compound that promotes mitochondrial fusion. In a specific embodiment, a composition of the disclosure comprises Mdivi-1 and M1.

### (b) Components of the Composition

The present disclosure also provides pharmaceutical compositions. The pharmaceutical composition comprises one or more compounds to inhibit mitochondrial fission and/or promote mitochondrial fusion, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980).

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising one or more compounds to inhibit mitochondrial fission and/or promote mitochondrial fusion is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of one or more compounds to inhibit mitochondrial fission and/or promote mitochondrial fusion in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the one or more compounds to inhibit mitochondrial fission and/or promote mitochondrial fusion may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying one or more compounds to inhibit mitochondrial fission and/or promote mitochondrial fusion may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the disclosure may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. one or more compounds to inhibit mitochondrial fission and/or promote mitochondrial fusion may be encapsulated in a microemulsion by any method generally known in the art.

In yet another embodiment, one or more compounds to inhibit mitochondrial fission and/or promote mitochondrial fusion may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### II. Methods

In an aspect, the disclosure provides a method of reducing immune cell activation in a subject. The method generally comprises administering to the subject a composition comprising one or more compounds to reduce mitochondrial fission or increase mitochondrial fusion. The method may comprise inner mitochondrial membrane remodeling; and detecting in the subject a marker for inflammation. More specifically, the method generally comprises administering to the subject a composition comprising one or more compounds to inhibit mitochondrial fission; and detecting in the subject a marker for inflammation. Non-limiting examples of immune cells include lymphocytes such as B cells (plasma cells and memory cells), T cells (T helper cells (T_{H} cells), cytotoxic or effector T cells (T_{C} cells, T_{E} cells or CTLs), memory T cells (T_{M} cells), suppressor T cells (T_{reg} cells), natural killer T cells (NKT cells), mucosal associated invariant T cells, and gamma delta T cells (γδ T cells)) and natural killer cells and granulocytes such as neutrophils, eosinophils, basophils, mast cells, and monocytes (dendritic cells and macrophages). In a specific embodiment, the immune cells are selected from the group consisting of T cells, macrophages and dendritic cells. Immune cell activation may be reduced by greater than 5% relative to a subject not administered a composition of the disclosure or relative to the same subject prior to administration of a composition of the disclosure. For example, immune cell activation may be reduced by greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, or greater than 95% relative to a subject not administered a composition of the disclosure or relative to the same subject prior to administration of a composition of the disclosure.

In another aspect, the disclosure provides a method of modulating the T cells subset numbers in a subject by administering to the subject a composition comprising one or more compounds to induce mitochondrial remodeling. The compounds that inhibit mitochondrial fission may reduce the number of inflammatory T cell subset and increase the regulatory T cell subset. Non-limiting examples of inflammatory T cell subset include T helper 1 (Th1), T helper 2 (Th2), and T helper 17 (Th17) T cell subset. In an aspect the compounds that inhibit mitochondrial fission may increase the number of Th17 cells. Th17 cells are inflammatory T cells and are characterized by production of inflammatory factors for example, but not limited to interleukins such as IL-17A, IL-17F, IL-21, and IL-22. The compounds that inhibit mitochondrial fission may decrease the differentiation of Th17, and decrease the number of Th17 cells. The compounds that inhibit mitochondrial fission may increase the number of Tregs. Treg are a regulatory T cell subset and have an immunosuppressive function. Treg may suppress the harmful effects of the helper T cells such as Th17 cells. Foxp3, a transcription factor is a marker for Tregs, and increase in Foxp3 expression may indicate an increase in Treg cells. Compounds that inhibit mitochondrial fission may increase Foxp3 expression. In an aspect, the compounds that mitochondrial fission may alter the balance between regulatory and Th17 T cells in favor of regulatory T cells.

In another aspect, the disclosure provides a method of reducing inflammation in a subject. The method generally comprises administering to the subject a composition comprising one or more compounds to induce inner mitochondrial membrane remodeling; and detecting in the subject a marker for inflammation. More specifically, the method generally comprises administering to the subject a composition comprising one or more compounds to inhibit mitochondrial fission; and detecting in the subject a marker for inflammation. Generally, inflammation is due to infiltration or activation of immune cells. The inflammation may be acute inflammation or chronic inflammation. As used herein, “acute inflammation” refers to inflammation that starts rapidly (rapid onset) and quickly becomes severe. Signs and symptoms are only present for a few days, but in some cases may persist for a few weeks. As used herein, “chronic inflammation” refers to long-term inflammation, which can last for several months and even years. Chronic inflammation may result from: failure to eliminate whatever was causing an acute inflammation, an autoimmune response to a self antigen, or a chronic irritant of low intensity that persists. Acute and chronic inflammation may lead to various diseases or disorders associated with inflammation. Non-limiting examples of diseases or disorders associated with inflammation include bronchitis, infected ingrown toenail, sore throat, scratch/cut, exercise, acne vulgaris, appendicitis, bursitis, dermatitis, eczema, cystitis, phlebitis, rhinitis, tonsillitis, meningitis, sinusitis, asthma, peptic ulcer, tuberculosis, periodontitis, pancreatitis, colitis, ulcerative colitis, Crohn's disease, inflammatory bowel disease, irritable bowel syndrome, diverticulitis, sinusitis, polymyalgia rheumatica, rheumatoid arthritis, lupus, psoriasis, psoriatic arthritis, gouty arthritis, osteoarthritis, fibromyalgia, tendonitis, scleroderma, atherosclerosis, vasculitis, hay fever, allergies, autoimmune diseases (for a non-limiting list of autoimmune diseases see www.aarda.org/autoimmune-information/list-of-diseases/), autoinflammatory diseases, celiac disease, prostatitis, nephritis, glomerulonephritis, kidney failure, hypersensitivities, pelvic inflammatory disease, reperfusion injury, sarcoidosis, transplant rejection, infection, sepsis, multiple sclerosis, obesity, non-alcoholic fatty liver disease, hepatitis, insulin resistance, diabetes, ankylosing spondylitis, anemia, autism, congestive heart failure, fibrosis, gall bladder disease, GERD, Guillain-Barre, Hashimoto's thyroiditis, heart attack, stroke, and surgical complications. In certain embodiments, a composition of the disclosure may be used to treat an inflammatory disease or disorder in which an induction of aerobic glycolysis correlates with disease state and/or progression, and an abrogation of this metabolism correlates with a better disease outcome. In a specific embodiment, the inflammation is due to obesity, rheumatoid arthritis, multiple sclerosis, Crohn's disease, irritable bowel disease, colitis, psoriasis, inflammatory liver disease, or nonalcoholic fatty liver disease.

Inflammation may be reduced by greater than 5% relative to a subject not administered a composition of the disclosure or relative to the same subject prior to administration of a composition of the disclosure. For example, inflammation may be reduced by greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, or greater than 95% relative to a subject not administered a composition of the disclosure or relative to the same subject prior to administration of a composition of the disclosure.

In still another aspect, the disclosure provides a method of treating sepsis in the subject. The method generally comprises administering to the subject a composition comprising one or more compounds to induce inner mitochondrial membrane remodeling; and detecting in the subject a marker for inflammation. More specifically, the method comprises administering to the subject a composition comprising one or more compounds to inhibit mitochondrial fission; and detecting in the subject a marker for inflammation. As used herein, the term “treating”, “treat” or “treatment” may mean to reduce or alleviate the signs or symptoms of disease, to prevent the signs or symptoms of disease, eliminate the signs or symptoms of disease, and/or to reduce, alleviate, prevent or eliminate the disease. Non-limiting examples of signs or symptoms of sepsis include fever, increased heart rate, increased breathing rate, and confusion. In certain embodiments, the method further comprises treating with standard treatment for sepsis. Non-limiting examples of standard treatments for sepsis include antibiotics and fluids (such as saline, albumin, dextran). Non-limiting examples of additional possible treatments for sepsis include corticosteroids, drotrecogin alfa, kidney dialysis, mechanical ventiliation, oxygen, and vasopressors.

According to the methods disclosed herein, a marker for inflammation is detected in the subject. In an embodiment, more than one marker for inflammation may be detected in the subject. For example, 1, 2, 3, 4, 5, 6, 7, 8, 9, 10, 11, 12, 13, 14, 15, 16, 17, 18, 19 or 20 or more markers for inflammation may be detected. As used herein, a “marker for inflammation” is any substance or feature that may be used to measure the degree of inflammation in a subject. For example, a marker for inflammation may be any substance or feature used to measure the degree of infiltration or activation of immune cells. Non-limiting examples of markers for inflammation include reduced glutathione (GSH) levels, reduced vitamin D and other antioxidants, elevated oxidized glutathione (GSSG) levels, elevated malondialdehyde (a marker for oxidative stress, formed when fats are oxidized), increased lipid peroxidation, elevated homocysteine, elevated c-reactive protein (CRP), elevated fructosamine, isoprostanes (a marker for oxidative stress, formed when fats are oxidized), elevated extracellular acidification rate (ECAR, an indicator of aerobic glycolysis), increased nitric oxide synthase 2 (Nos2) protein expression, increased immune cells (examples of immune cells are described above), elevated lactate, elevated pro-inflammatory cytokines, repressed anti-inflammatory cytokines, altered mitochondrial structure, altered mitochondrial function, increased erythrocyte sedimentation rate (ESR), increased plasma viscosity (PV), pain, heat, redness, swelling, and loss of function. In certain embodiments, a marker for inflammation is selected from the group consisting of cytokines, immune cells, lactate, C-reactive protein (CRP), mitochondrial structure, mitochondrial function, and metabolic function of immune cells. The term “pro-inflammatory cytokine” is a cytokine which promotes systemic inflammation. A skilled artisan would be able to determine those cytokines that are pro-inflammatory. Non-limiting examples of pro-inflammatory cytokines include IL-1α, IL-1β, IL-1Ra, IL-2, IL-3, IL-6, IL-7, IL-9, IL-12, IL-15, IL-17, IL-18, IL-21, IL-23, IL-33, IL-36Ra, IL-36α, IL-36β, IL-36γ, IL-37, IL-38, IFN-α, IFN-γ, TNF-α, MIF, iNOS, Cox-2, G-CSF, and GM-CSF. The term “anti-inflammatory cytokine” is a cytokine that counteracts various aspects of inflammation, for example cell activation or the production of pro-inflammatory cytokines, and thus contributes to the control of the magnitude of the inflammatory response. A skilled artisan would be able to determine those cytokines that are anti-inflammatory. Non-limiting examples of anti-inflammatory cytokines include IL-4, IL-5, IL-10, IL-11, IL-13, IL-16, IL-35, IFN-α, TGF-β, and G-CSF. The cytokine to be detected may be chosen based on the specific inflammatory disease or condition.

In a subject experiencing inflammation, a marker of inflammation may be altered following administration of a composition of the disclosure. For example, if the marker of inflammation is increased or present during inflammation, then administration a composition of the disclosure reduces, alleviates or eliminates the marker of inflammation. Alternatively, if the marker of inflammation is decreased or absent during inflammation, then administration of a composition of the disclosure increases the marker of inflammation. An increase or decrease may be measured relative to a subject not administered a composition of the disclosure or relative to the same subject prior to administration of a composition of the disclosure. In a specific embodiment, extracellular acidification rate (ECAR) is measured as a marker of inflammation. In such an embodiment, ECAR is suppressed in immune cells from a subject following administration of a composition of the disclosure. For example, ECAR may be suppressed about 5% or more in immune cells from a subject following administration of a composition of the disclosure relative to immune cells from a subject not administered a composition of the disclosure or relative to immune cells from the same subject prior to administration of a composition of the disclosure. For example, ECAR may be suppressed about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, or about 95% or more in immune cells from a subject following administration of a composition of the disclosure relative to immune cells from a subject not administered a composition of the disclosure or relative to immune cells from the same subject prior to administration of a composition of the disclosure. In another specific embodiment, nitric oxide synthase 2 (Nos2) protein expression is measured as a marker of inflammation. In such an embodiment, Nos2 protein expression is suppressed in immune cells from a subject following administration of a composition of the disclosure. For example, Nos2 protein expression may be suppressed about 1.2-fold or more in immune cells from a subject following administration of a composition of the disclosure relative to immune cells from a subject not administered a composition of the disclosure or relative to immune cells from the same subject prior to administration of a composition of the disclosure. For example, Nos2 protein expression may be suppressed about 1.2-fold or more, about 1.3-fold or more, about 1.4-fold or more, about 1.5-fold or more, about 1.6-fold or more, about 1.7-fold or more, about 1.8-fold or more, about 1.9-fold or more, about 2-fold or more, about 3-fold or more, about 4-fold or more, about 5-fold or more, about 6-fold or more, about 7-fold or more, about 8-fold or more, about 9-fold or more, about 10-fold or more, about 15-fold or more, about 20-fold or more, about 25-fold or more, about 30-fold or more, about 35-fold or more, about 40-fold or more, about 45-fold or more, about 50-fold or more, about 100-fold or more, about 200-fold or more, about 500-fold or more, or about 1000-fold or more in immune cells from a subject following administration of a composition of the disclosure relative to immune cells from a subject not administered a composition of the disclosure or relative to immune cells from the same subject prior to administration of a composition of the disclosure.

The marker of inflammation may be measured visually by inspecting the subject or imaging the subject. Methods of imaging a subject to detect markers of inflammation are known in the art. For example, imaging techniques may include ultrasonography, CT, MRI, endoscopic techniques, PET, planar scintigraphy, and SPECT. For methods of imaging inflammation, see for example Gotthardt et al. J Nucl Med 2010; 51: 1937-1949, the disclosure of which is hereby incorporated by reference in its entirety. A subject may be visually inspected for the presence of heat, redness, and swelling.

Alternatively, the marker of inflammation may be measured in a biological sample obtained from the subject. As used herein, the term “biological sample” refers to a sample obtained from a subject. Any biological sample containing a marker of inflammation is suitable. Numerous types of biological samples are known in the art. Suitable biological samples may include, but are not limited to, tissue samples or bodily fluids. In some embodiments, the biological sample is a tissue sample such as a tissue biopsy. The biopsied tissue may be fixed, embedded in paraffin or plastic, and sectioned, or the biopsied tissue may be frozen and cryosectioned. In other embodiments, the sample may be a bodily fluid. Non-limiting examples of suitable bodily fluids include blood, plasma, serum, peripheral blood, bone marrow, urine, saliva, sputum, and cerebrospinal fluid. In a specific embodiment, the biological sample is blood, plasma, serum. In another specific embodiment, the biological sample is blood. The fluid may be used “as is”, the cellular components may be isolated from the fluid, or a nucleic acid or protein fraction may be isolated from the fluid using standard techniques.

As will be appreciated by a skilled artisan, the method of collecting a biological sample can and will vary depending upon the nature of the biological sample and the type of analysis to be performed. Any of a variety of methods generally known in the art may be utilized to collect a biological sample. Generally speaking, the method preferably maintains the integrity of the sample such that the marker of inflammation can be accurately detected and the amount measured according to the disclosure.

In some embodiments, a single sample is obtained from a subject to detect a marker of inflammation in the sample. Alternatively, a marker of inflammation may be detected in samples obtained over time from a subject. As such, more than one sample may be collected from a subject over time. For instance, 2, 3, 4, 5, 6, 7, 8, 9, 10, 11, 12, 13, 14, 15, 16 or more samples may be collected from a subject over time. In some embodiments, 2, 3, 4, 5, or 6 samples are collected from a subject over time. In other embodiments, 6, 7, 8, 9, or 10 samples are collected from a subject over time. In yet other embodiments, 10, 11, 12, 13, or 14 samples are collected from a subject over time. In other embodiments, 14, 15, 16 or more samples are collected from a subject over time.

When more than one sample is collected from a subject over time, samples may be collected every 1, 2, 3, 4, 5, 6, 7, 8, 9, 10, 11, 12 or more hours. In some embodiments, samples are collected every 1, 2, 3, 4, or 5 hours. In other embodiments, samples are collected every 5, 6, 7, 8, or 9 hours. In yet other embodiments, samples are collected every 9, 10, 11, 12 or more hours. Alternatively, when more than one sample is collected from a subject over time, samples may be collected every 1, 2, 3, 4, 5, 6, 7, 8, 9, 10, 11, 12 or more days. In some embodiments, samples are collected every 1, 2, 3, 4, or 5 days. In other embodiments, samples are collected every 5, 6, 7, 8, or 9 days. In yet other embodiments, samples are collected every 9, 10, 11, 12 or more days. In still other embodiments, samples are collected a month apart, 3 months apart, 6 months apart, 1 year apart, 2 years apart, 5 years apart, 10 years apart or more.

As used herein, “subject” or “patient” is used interchangeably. Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In specific embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In a preferred embodiment, the subject is human.

### (a) Administration

In certain aspects, a therapeutically effective amount of a composition of the disclosure may be administered to a subject. Administration is performed using standard effective techniques, including peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

Specific methods of administration may include transporter facilitated drug uptake. In such an embodiment, a metabolite may be conjugated to a compound of the disclosure to enhance delivery of the compound to a specific target cell population. For example, a metabolite that binds to immune cells may be conjugated to a compound of the disclosure to enhance delivery of the compound to immune cells. Additionally, administration may include controlled or sustained release of a compound of the disclosure from biodegradable nanospheres. In such an embodiment, a compound of the disclosure may be encapsulated in a biodegradable nanosphere (e.g. PLGA nanoparticles). In further embodiments, the compound loaded nanospheres may be further conjugated to a specific antibody for specific delivery to a target cell population. For example, an antibody that binds to immune cells may be conjugated to the nanospheres for targeted delivery to immune cells. Non-limiting examples of antibodies include anti-CD8.

For therapeutic applications, a therapeutically effective amount of a composition of the disclosure is administered to a subject. A “therapeutically effective amount” is an amount of the therapeutic composition sufficient to produce a measurable response (e.g., reduced immune cell activation, reduced inflammation, change in a marker of inflammation). Actual dosage levels of active ingredients in a therapeutic composition of the disclosure can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, age, the inflammatory disease, the symptoms, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The frequency of dosing may be once, twice, three times or more daily or once, twice, three times or more per week or per month, as needed as to effectively treat the symptoms or disease. In certain embodiments, the frequency of dosing may be once, twice or three times daily. For example, a dose may be administered every 24 hours, every 12 hours, or every 8 hours. In other embodiments, the frequency of dosing may be once, twice or three times weekly. For example, a dose may be administered every 2 days, every 3 days or every 4 days. In a different embodiment, the frequency of dosing may be one, twice, three or four times monthly. For example, a dose may be administered every 1 week, every 2 weeks, every 3 weeks or every 4 weeks.

Duration of treatment could range from a single dose administered on a one-time basis to a life-long course of therapeutic treatments. The duration of treatment can and will vary depending on the subject and the inflammation to be treated. For example, the duration of treatment may be for 1 day, 2 days, 3 days, 4 days, 5 days, 6 days, or 7 days. Or, the duration of treatment may be for 1 week, 2 weeks, 3 weeks, 4 weeks, 5 weeks or 6 weeks. Alternatively, the duration of treatment may be for 1 month, 2 months, 3 months, 4 months, 5 months, 6 months, 7 months, 8 months, 9 months, 10 months, 11 months, or 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, duration of treatment may be 5 days, then no treatment for 9 days, then treatment for 5 days.

Typical dosage levels can be determined and optimized using standard clinical techniques and will be dependent on the mode of administration. For example, compounds of the disclosure may be administered at doses ranging from about 0.1 mg/kg to about 500 mg/kg. For example, the dose of compounds of the disclosure may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of compounds of the disclosure may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of compounds of the disclosure may be about 300 mg/kg, about 325 mg/kg, about 350 mg/kg, about 375 mg/kg, about 400 mg/kg, about 425 mg/kg, about 450 mg/kg, about 475 mg/kg or about 500 mg/kg.

The method may further comprise administration of agents standard in the art for treating inflammation. Such agents may depend on the type and severity of inflammation, as well as the general condition of the patient. Non-limiting examples of treatment of inflammation include administration of anti-inflammatory pain reliever drugs (NSAIDs such as aspirin, ibuprofen, naproxen, or Celebrex), acetaminophen, corticosteroids (such as prednisone), immune selective anit-inflammatory derivatives (ImSAIDS) and other medications such as chemotherapy, disease modifying treatments, biologic therapy, narcotic pain relievers, or herbs (such as Harpagophytum procumbens, Hyssop Hyssopus, ginger, turmeric, cannabis), heat therapy, cryotherapy, fish oil, green tea, tart cherries, electrical stimulation, traction, massage, and acupuncture. Additionally, see patient.info/medicine/medicines-used-to-treat-inflammation-1281 for a list of medicines used to treat inflammation. Additional, the method may further comprise administration of agents standard in the art for treating the inflammatory disease or condition.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to the Examples.

T cells are important mediators of protective immunity against pathogens and cancer and have several unique properties, not least of which is their ability to proliferate at a rate arguably unlike any other cell in an adult organism. In this regard, one naïve T cell can clonally expand into millions of ‘armed’ T_{E} cells in just a few days (Williams and Bevan, 2007). Concomitant with T cell activation is the engagement of Warburg metabolism, a metabolic phenotype shared by cancer cells and unicellular organisms (Fox et al., 2005, Vander Heiden et al., 2009). Once the source of antigen is cleared, most antigen-specific cells die, but a subset of long-lived, resting T_{M} cells persists (Kaech et al., 2003). T_{M} cells have a unique metabolism that renders them equipped to rapidly respond should infection or tumor growth recur (Pearce et al., 2013). These extensive changes in phenotype and function of T cells go hand in hand with a highly dynamic metabolic range (MacIver et al., 2013, Buck et al., 2015). As such, these cells represent a distinctive and amenable system in which we can study marked changes in cellular metabolism that occur as part of normal cellular development, and not as a result of transformation.

Both OXPHOS and aerobic glycolysis generate energy in the form of ATP, but importantly, are also critical for other essential processes such as the building of biosynthetic precursors for biomass, the production of reactive oxygen species (ROS), and the balance of reducing/oxidizing equivalents like NADH/NAD⁺ which take part in redox reactions that release energy from nutrients. Naïve T (T_{N}) cells use OXPHOS for their metabolic needs, but both OXPHOS and aerobic glycolysis are augmented upon activation (Chang et al., 2013, Sena et al., 2013). The latter is characterized by the preferential conversion of pyruvate to lactate in the cytoplasm rather than its oxidation in the TCA cycle. T_{M} cells predominantly use OXPHOS like T_{N} cells, but have enhanced mitochondrial capacity that is marked by their reliance on FAO to fuel OXPHOS (van der Windt et al., 2012, van der Windt et al., 2013). Failure to engage specific metabolic programs impairs the function and differentiation of T cells (Pearce and Pearce, 2013). Establishing the precise reasons why, and how, these and other cells emphasize one particular metabolic pathway over another remains a challenging prospect.

Mitochondria are essential hubs of metabolic activity, antiviral responses, and cell death (Nunnari and Suomalainen, 2012). These dynamic organelles constantly remodel their structure through fission and fusion events mediated by highly conserved nuclear encoded GTPases (Youle and van der Bliek, 2012, Ishihara et al., 2013). Mitochondrial fission generates smaller, discrete and fragmented mitochondria that can increase ROS production (Yu et al., 2006), facilitate mitophagy (Frank et al., 2012), accelerate cell proliferation (Taguchi et al., 2007, Marsboom et al., 2012), and mediate apoptosis (Youle and Karbowski, 2005). Dynamin-related protein 1 (Drp1) is a cytosolic protein that translocates to the outer mitochondrial membrane (OMM) upon phosphorylation to scission mitochondria (Labrousse et al., 1999, Ingerman et al., 2005, Wakabayashi et al., 2009). Fusion of mitochondria into linear or tubular networks limits deleterious mutations in mitochondrial DNA (mtDNA) (Santel et al., 2003), induces supercomplexes of the ETC maximizing OXPHOS activity (Zorzano et al., 2010, Cogliati et al., 2013, Mishra et al., 2014), and enhances endoplasmic reticulum (ER) interactions important for calcium flux (de Brito and Scorrano, 2008). In addition, mitochondria elongate as a survival mechanism in response to nutrient starvation and cell stress, linking fusion to cellular longevity (Gomes et al., 2011, Rambold et al., 2011a, Friedman and Nunnari, 2014). OMM fusion is mediated by mitofusin 1 and 2 (Mfn1, Mfn2) isoforms (Chen et al., 2003), while inner membrane fusion is controlled by optic atrophy 1 (Opa1) protein (Cipolat et al., 2004). Complete organismal deficiency in any of these proteins is embryonically lethal and mutations in the genes that encode them underlie the cause of several human diseases (Chen et al., 2007, Zhang et al., 2011, Chan, 2012, Archer, 2014).

Mitochondrial membrane remodeling has been largely demonstrated to be acutely responsive to changes in cellular metabolism (Mishra and Chan, 2016, Wai and Langer, 2016), but whether it plays a dynamic role in shaping metabolic pathways has been inferred but not extensively studied. At the cellular level, deletion of any of the fission and fusion machinery perturbs OXPHOS and glycolytic rates at baseline (Liesa and Shirihai, 2013). Tissue-specific deletion of Mfn2 in muscles of mice disrupts glucose homeostasis (Sebastian et al., 2012) and Drp1 ablation in the liver results in reduced adiposity and elevated whole-body energy expenditure, protecting mice from diet-induced obesity (Wang et al., 2015). A recent study has also suggested a link between Drp1 mediated fission and its effect on glycolysis during cell transformation (Serasinghe et al., 2015). The central question of whether fission/fusion and associated changes in cristae morphology actively control the adoption of distinct metabolic programs and therefore regulates T cell responses however, remains unanswered.

### Example 1. Unlike T_{E} Cells, T_{M} Cells Maintain a Fused Mitochondrial Network

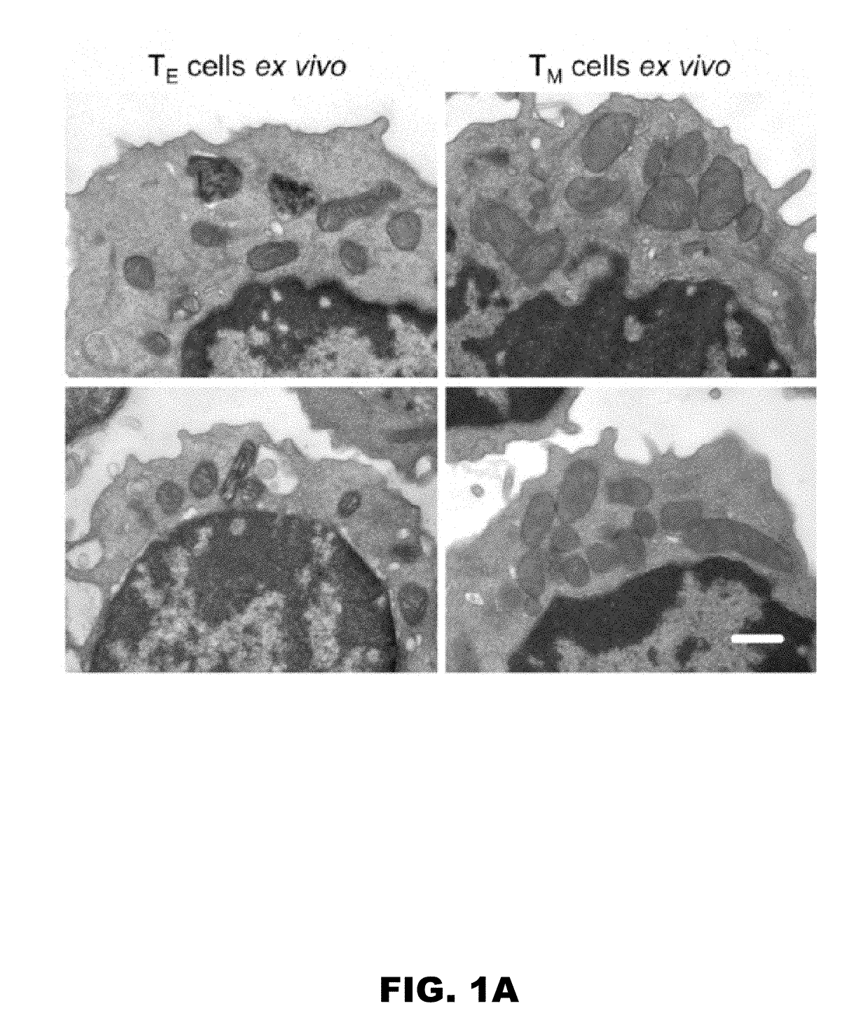

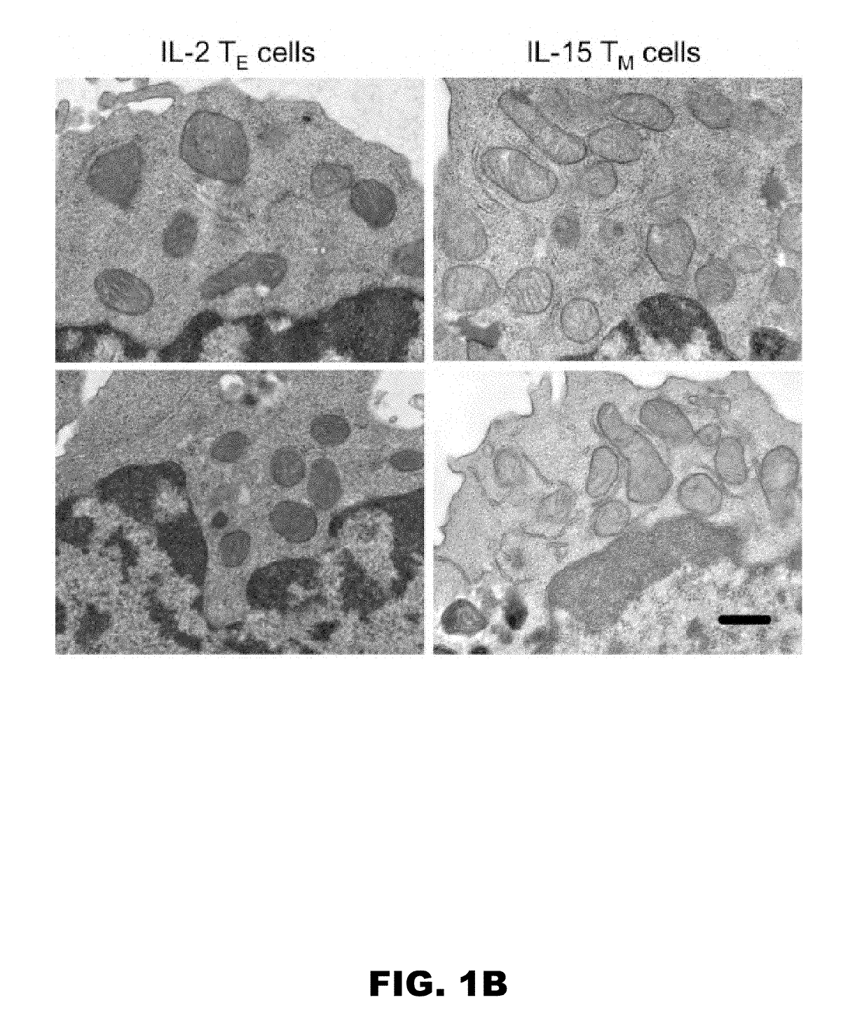

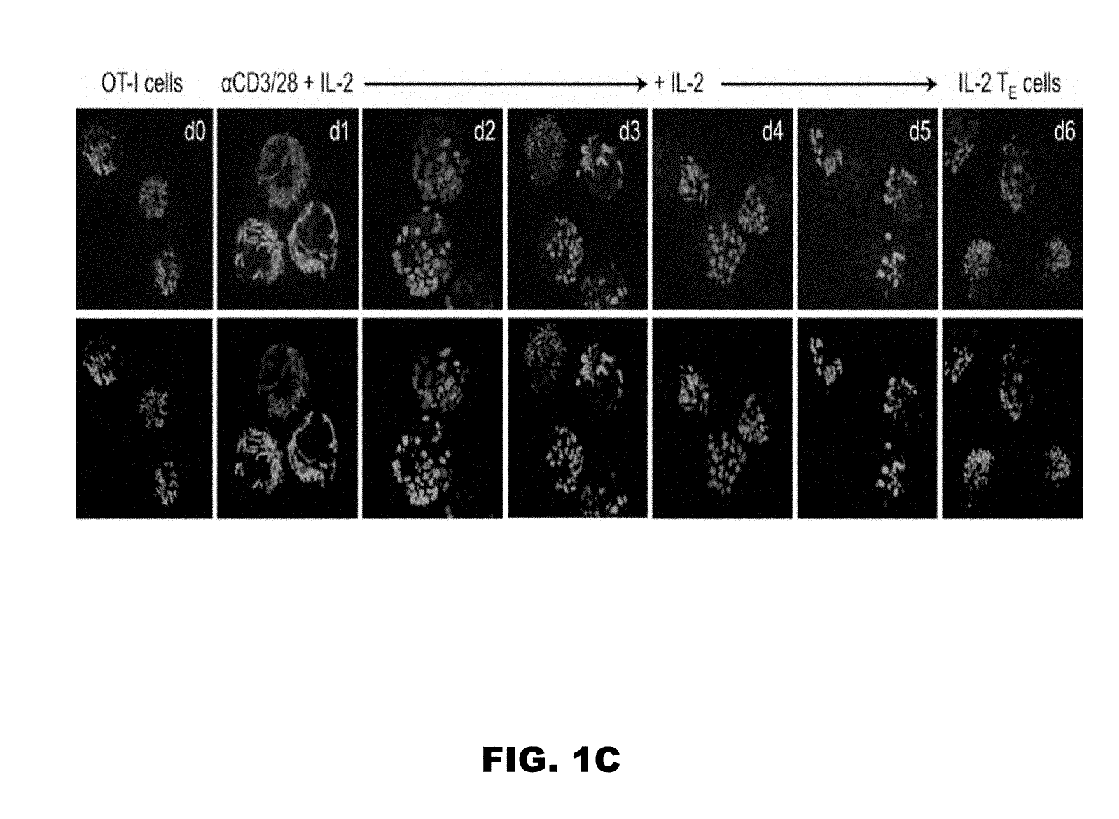

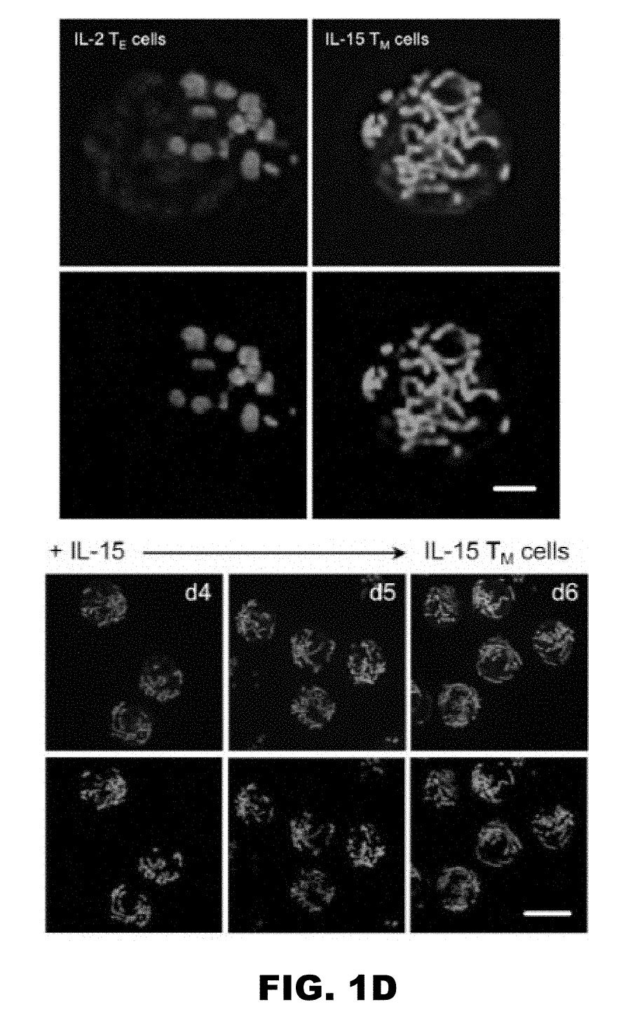

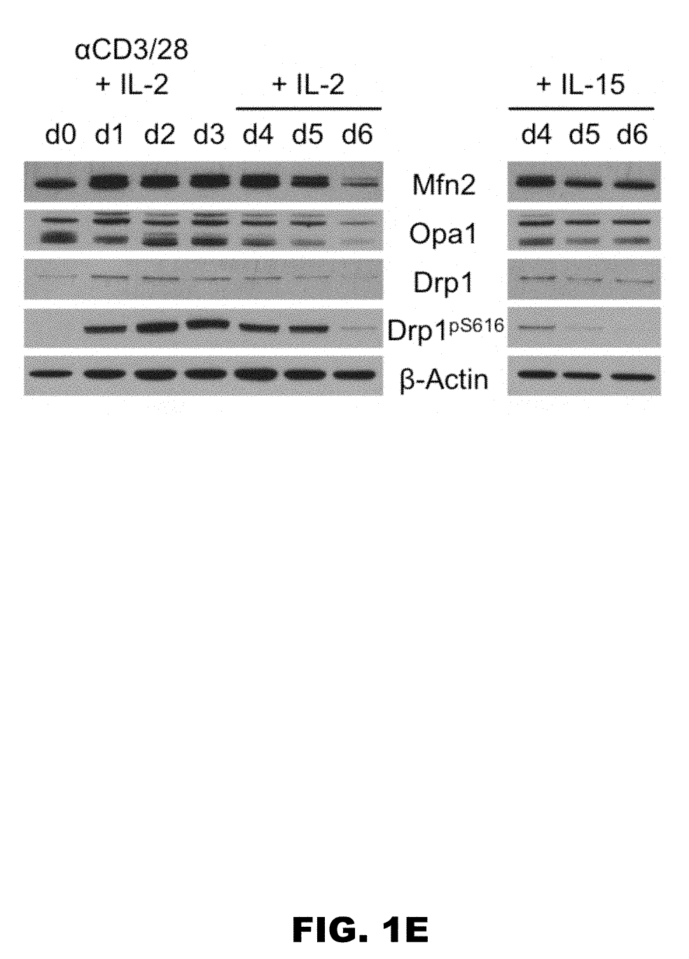

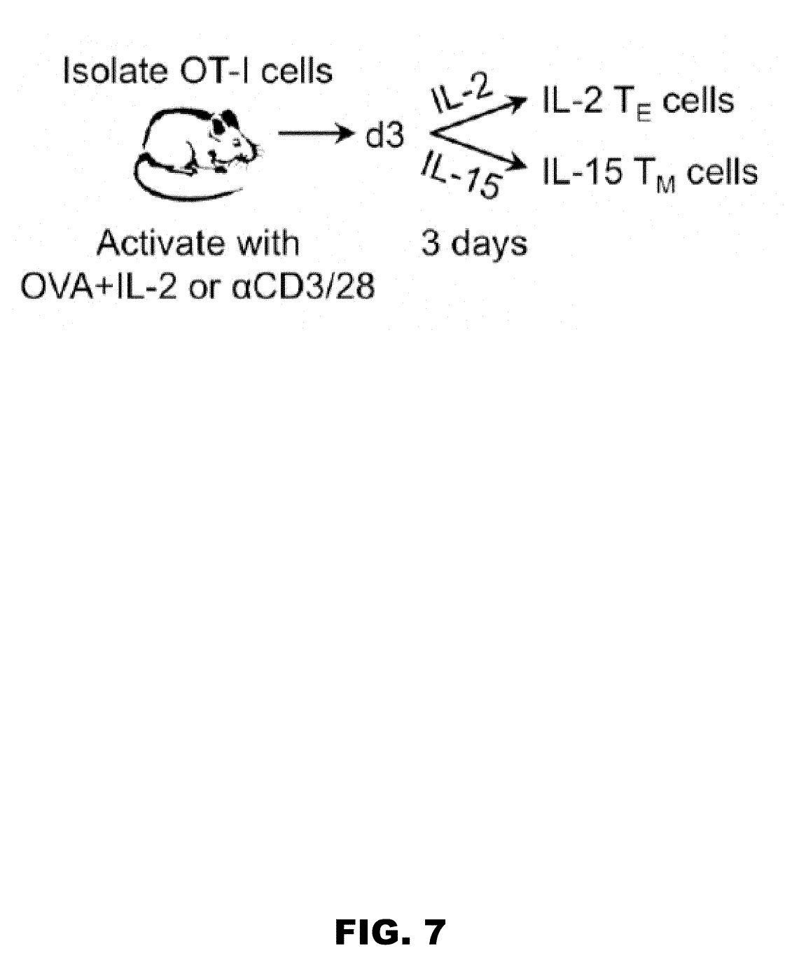

T_{M} cells have more mitochondrial mass than T_{E} or T_{N} cells and suggested that mitochondria in these T cell subsets possess distinct morphologies. These observations prompted us to more closely assess mitochondrial structure in T cells. We infected mice with Listeria monocytogenes expressing ovalbumin (OVA) (LmOVA) and isolated T_{E} and T_{M} cells for ultrastructure analysis by electron microscopy (EM). We found that T_{E} cells had small, distinct mitochondria dispersed in the cytoplasm, while T_{M} cells had more densely packed, somewhat tubular, mitochondria (FIG. 1A). In order to more thoroughly investigate these morphological differences, we differentially cultured activated OVA-specific T cell receptor (TCR) transgenic OT-I cells in interleukin-2 (IL-2) and IL-15 to generate IL-2 T_{E} and IL-15 T_{M} cells (FIG. 7) (Carrio et al., 2004). These culture conditions approximate T cell responses in vivo and allow us to generate large numbers of cells amenable to further experimentation in vitro (O'Sullivan et al., 2014). We found that IL-2 T_{E} and IL-15 T_{M} cells possessed similar mitochondrial ultrastructure as their ex vivo isolated counterparts (FIG. 1B). Next, we acquired live Z-stacked images of these T cells over time by confocal microscopy and found that while at day 1 after activation the mitochondria appeared fused, from days 2-6 after activation, IL-2 T_{E} cells exhibited predominantly punctate mitochondria (FIG. 1C). In contrast, once cells were exposed to IL-15, a cytokine that supports TM cell formation (Schluns et al., 2002), the mitochondria formed elongated tubules (FIG. 1C). Magnified 3D rendered images from these experiments emphasized the marked differences in mitochondrial morphology between the IL-2 T_{E} and IL-15 T_{M} cells (FIG. 1D). Together these data suggest that the mitochondria in T_{E} cells are actively undergoing fission, while in T_{M} cells, these organelles exist in a fused state. To further investigate these changes in mitochondrial morphology, we assessed the expression of several critical protein regulators of mitochondrial dynamics. We found that by day 6, fusion mediators Mfn2 and Opa1 were lower in T_{E} cells compared to T_{M} cells, while fission factor Drp1 was more highly phosphorylated at its activating site Ser616 in T_{E} cells (FIG. 1E) (Marsboom et al., 2012). These data are consistent with our observations that mitochondria in T_{M} cells appear more fused than those in T_{E} cells.

### Example 2. Mitochondrial Inner Membrane Fusion Protein Opa1 is Necessary for T_{M} Cell Generation

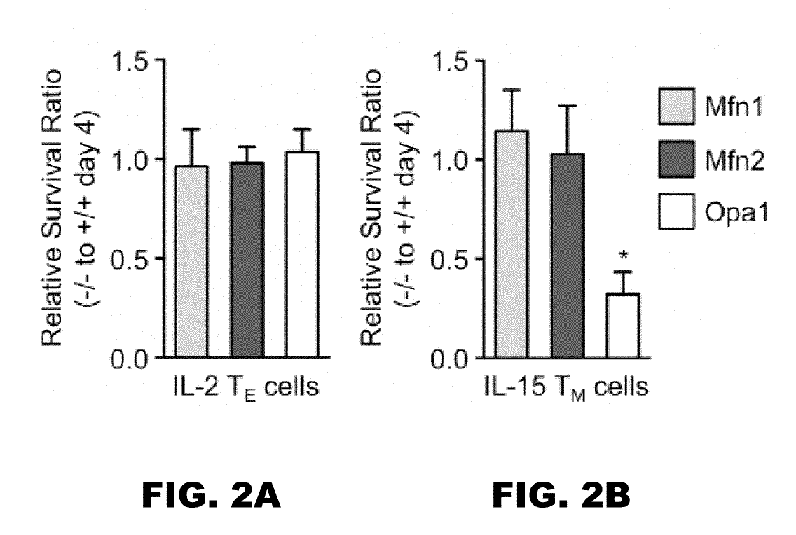

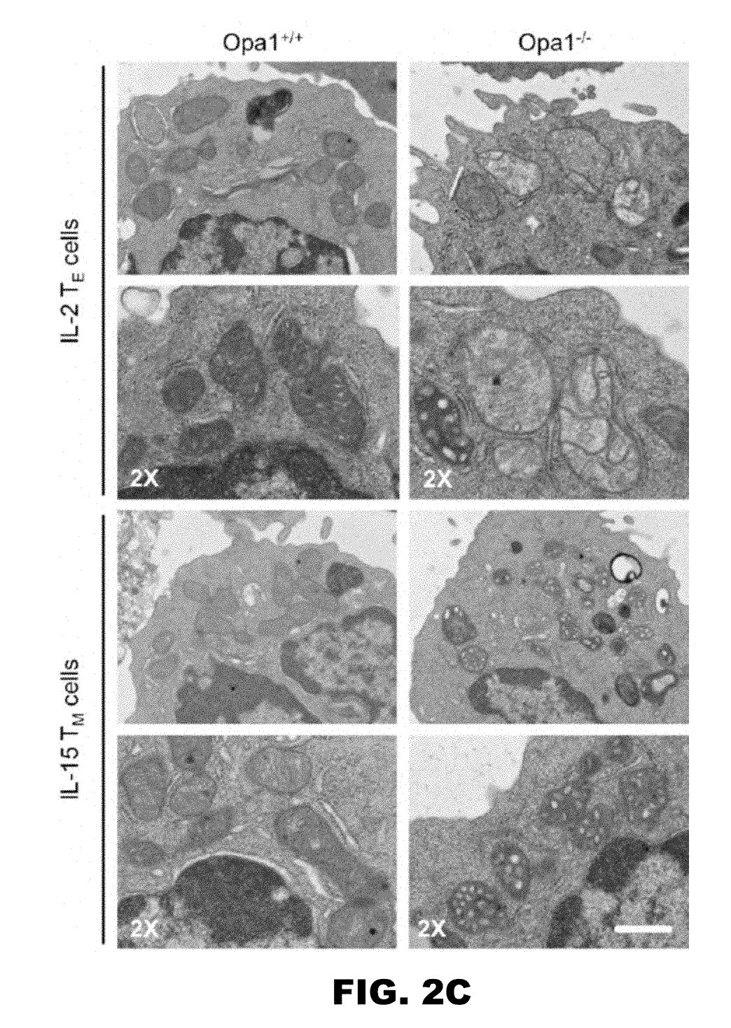

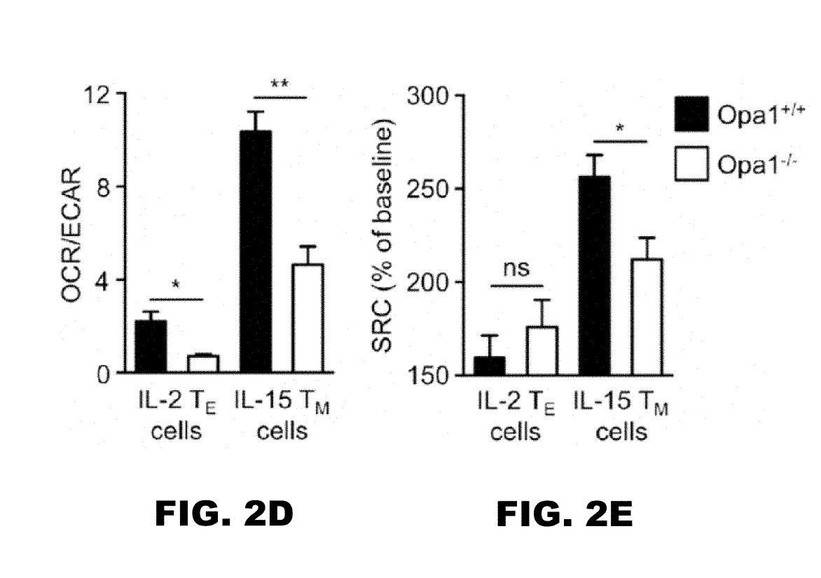

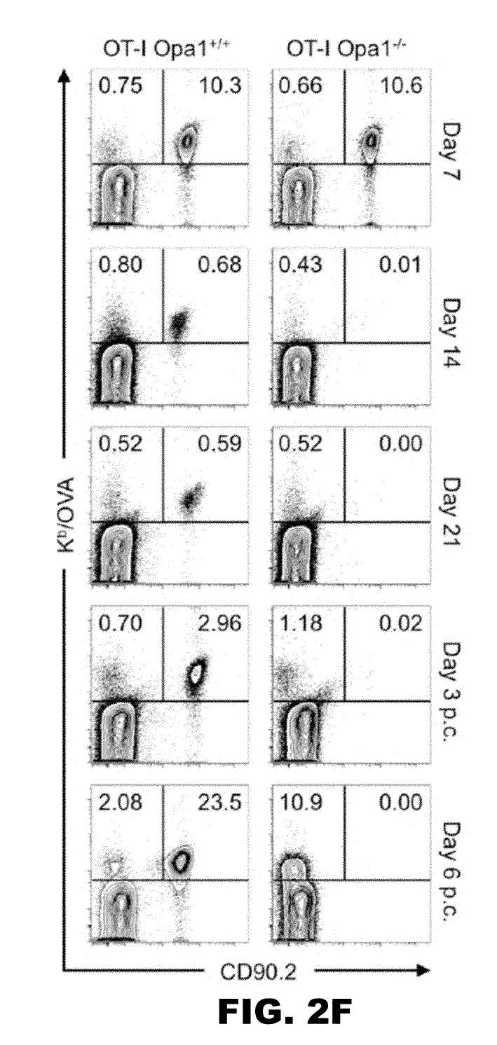

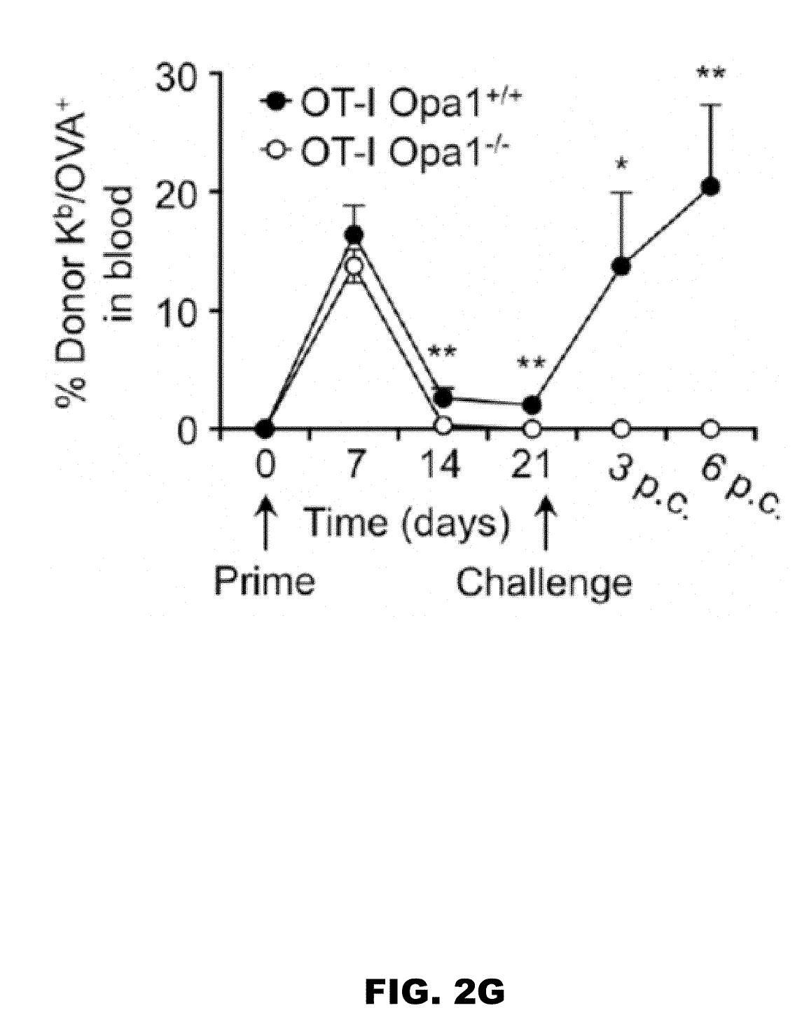

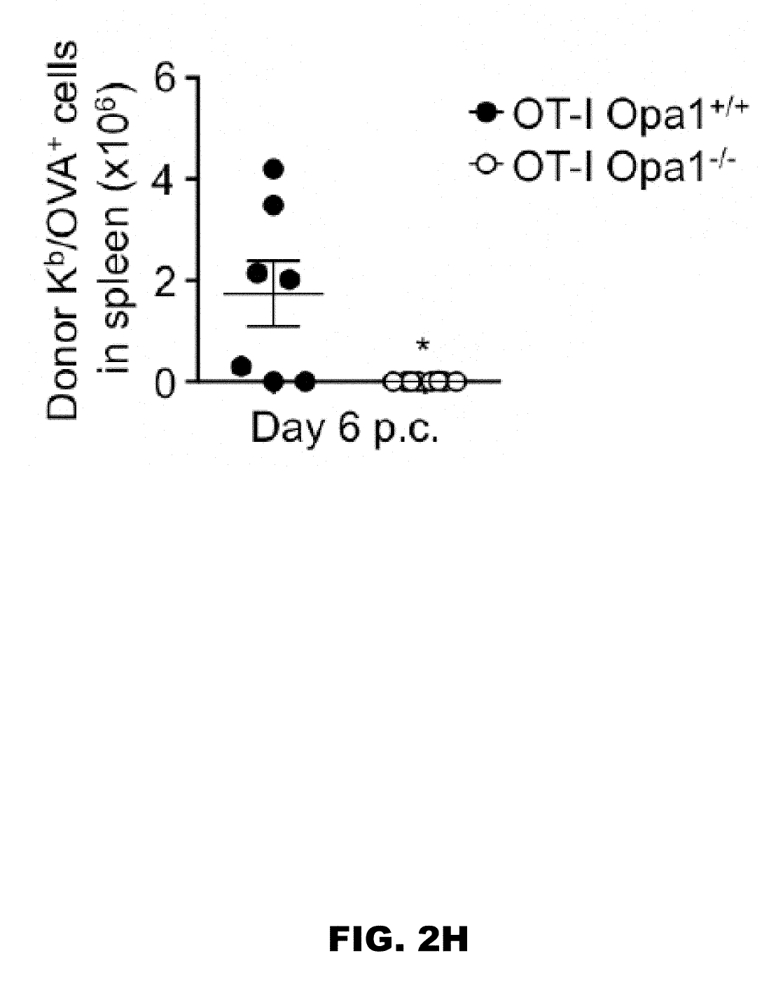

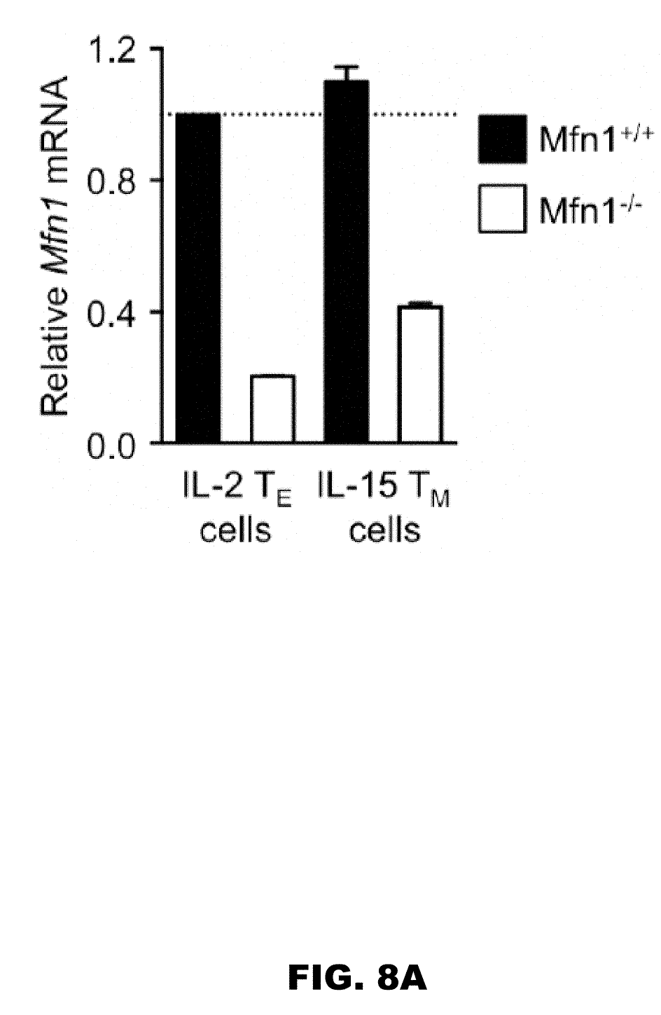

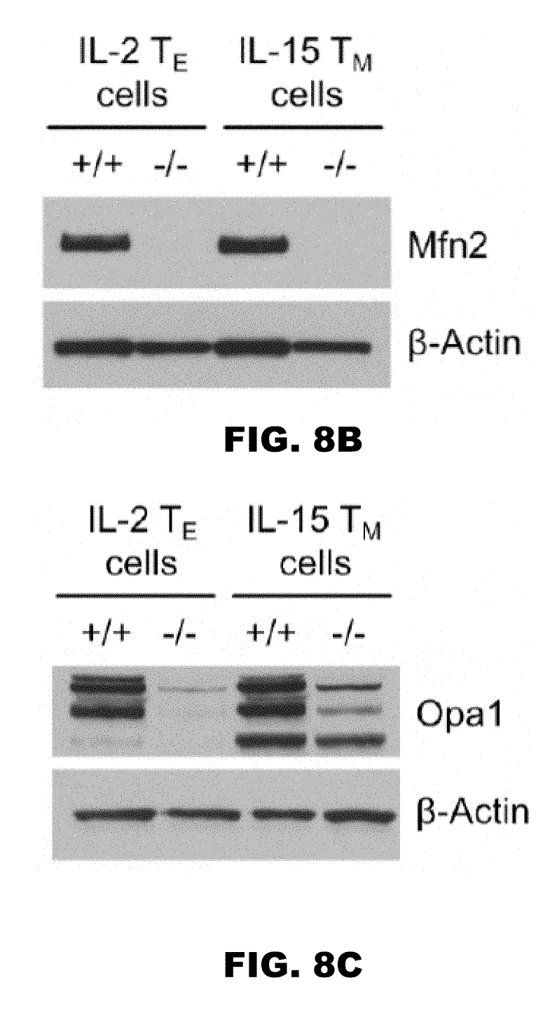

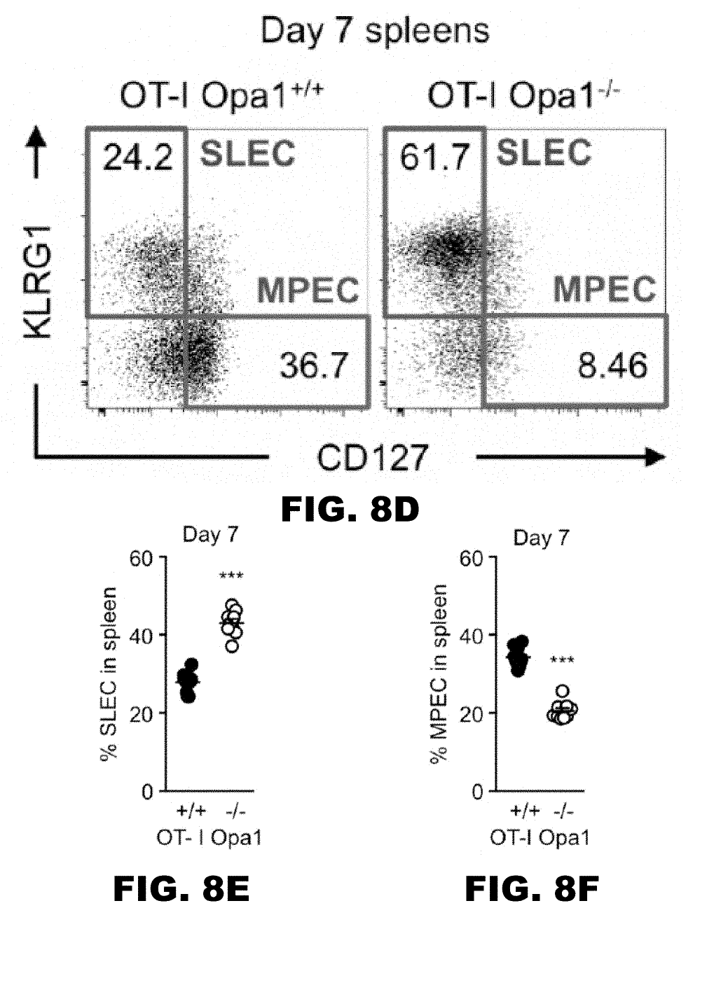

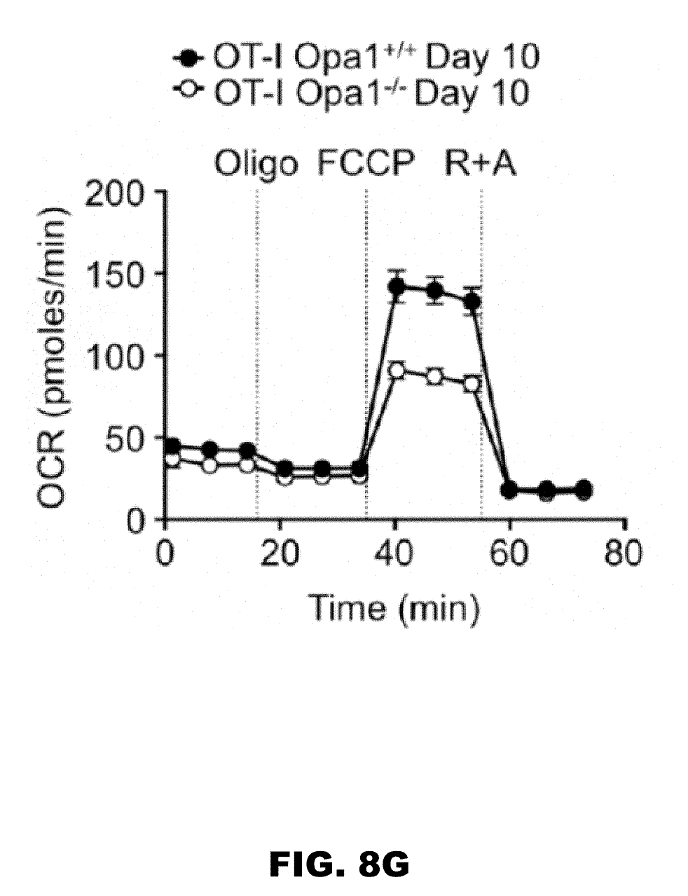

We questioned next whether mitochondrial fusion was important for T_{M} cell generation and survival. We crossed Mfn1, Mfn2, and Opa1 floxed mice to OT-I CD4 Cre transgenic mice to conditionally delete these proteins in T cells. Peripheral T cell frequencies in these mice were grossly normal (data not shown). We differentially cultured these Mfn1^{-/-}, Mfn2^{-/-}, and Opa1^{-/-} OT-I T cells in IL-2 and IL-15 and found that only Opa1^{-/-} T cells displayed a selective defect in survival when cultured in IL-15 (FIG. 2A, FIG. 2B). Opa1 deficiency did not effect IL-2 T_{E} cell survival. We measured the efficiency of gene deletion by mRNA and/or protein analyses (FIG. 8A, FIG. 8B, FIG. 8C). While Mfn1 and 2 were efficiently deleted, we found some residual expression of Opa1 particularly in IL-15 T_{M} cells, suggesting that we were selecting for cells that retained expression of Opa1 in IL-15 culture conditions, albeit at a diminished level as most of these cells die (FIG. 2A, FIG. 2B). We also assessed mitochondrial ultrastructure and, in agreement with published results for other cell types (Zhang et al., 2011, Cogliati et al., 2013), mitochondrial cristae were significantly altered and disorganized in the absence of Opa1 (FIG. 2C). Consistent with their survival defect, Opa1^{-/-} IL-15 T_{M} cells exhibited decreased OXPHOS activity, as measured by 02 consumption rate (OCR, an indicator of OXPHOS) to extracellular acidification rate (ECAR, an indicator of aerobic glycolysis) ratio, and spare respiratory capacity (SRC), compared to normal cells (FIGS. 2D and 2E). SRC is the extra mitochondrial capacity available in a cell to produce energy under conditions of increased work or stress and is thought to be important for long-term cellular survival and function (measured as OXPHOS activity above basal after uncoupling with FCCP) (Yadava and Nicholls, 2007, Ferrick et al., 2008, Choi et al., 2009, Nicholls, 2009, Nicholls et al., 2010, van der Windt et al., 2012). To determine whether Opa1 function is required for T_{M} cell development in vivo, we adoptively transferred naïve Opa1^{-/-} OT-I T cells into congenic recipients, infected these mice with LmOVA, and subsequently assessed T_{M} cell formation in the weeks after infection. Control and Opa1^{-/-} OT-I T cells mounted normal T_{E} cell responses (day 7) to infection, while Opa1^{-/-} OT-I T_{M} cell formation (days 14-21) was drastically impaired (FIG. 2F). Consistent with diminished T_{M} cell development, a significantly higher proportion of short-lived effector cells to memory precursor effector cells were present within the Opa1^{-/-} OT-I donor cell population 7 days after infection (FIG. 8D, FIG. 8E, FIG. 8F) (Kaech et al., 2003). In addition, at day 10 post-infection, a time point at which T_{E} cells contract, while T_{M} cells emerge, Opa1^{-/-} T cells isolated ex vivo had decreased SRC compared to control cells (FIG. 8G), correlating with their decreased survival. To assess whether Opa1^{-/-} T_{M} cells existed in too low an abundance to be discerned by flow cytometry, we challenged these mice with a second infection. We observed no recall response (day 3 and 6 p.c.) from Opa1^{-/-} T cells when assessing frequency (FIG. 2G) or absolute numbers (FIG. 2H), while there was considerable expansion of control donor cells. These data illustrate that Opa1 function is required for T_{M} cell, but not T_{E} cell generation.

### Example 3. Mitochondrial Fusion Imposes a T_{M} Cell Phenotype, Even in the Presence of Activating Signals

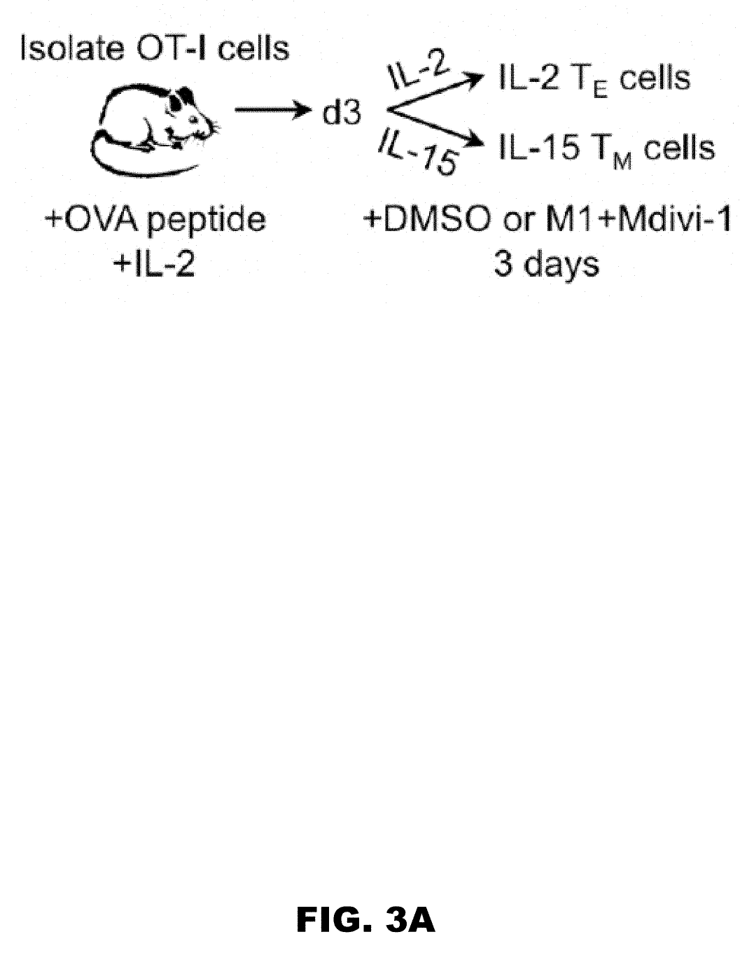

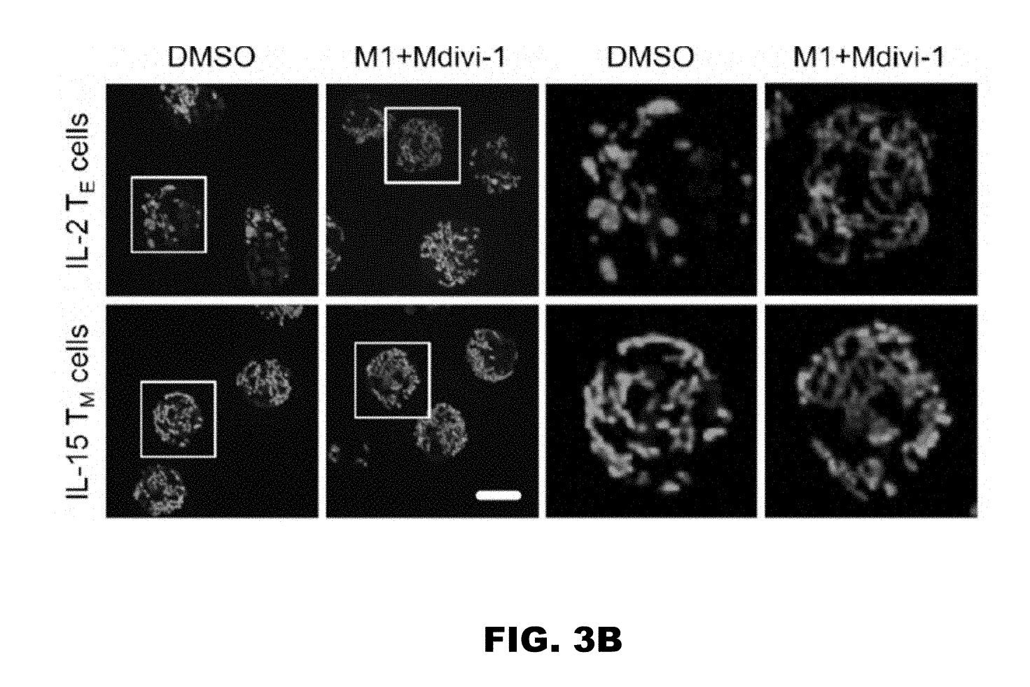

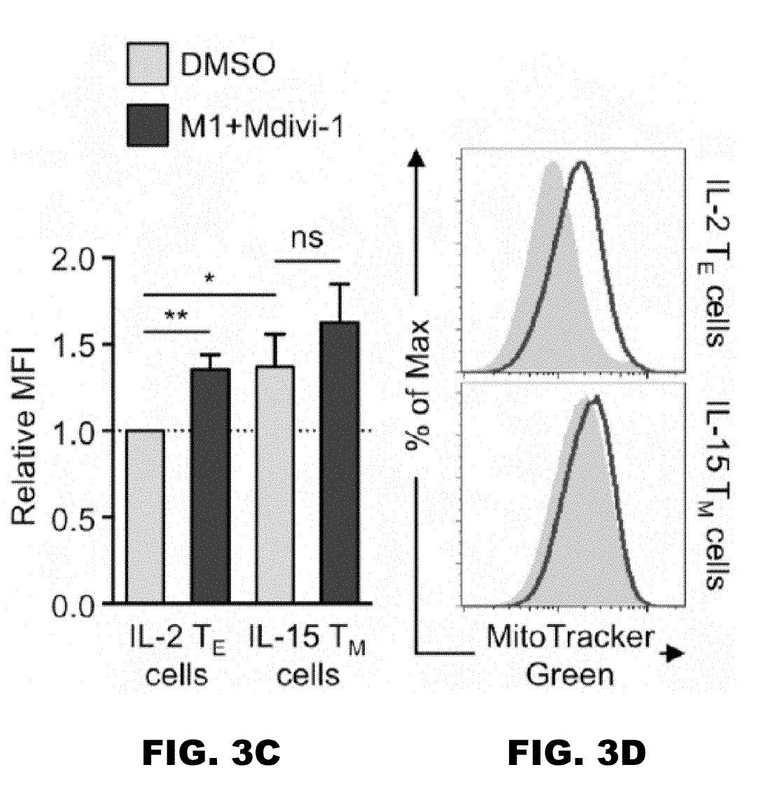

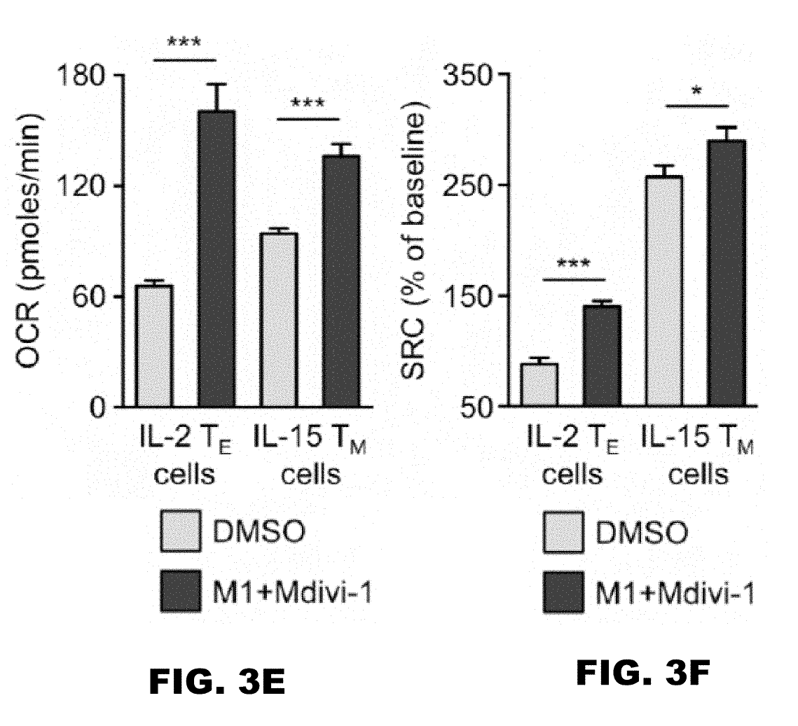

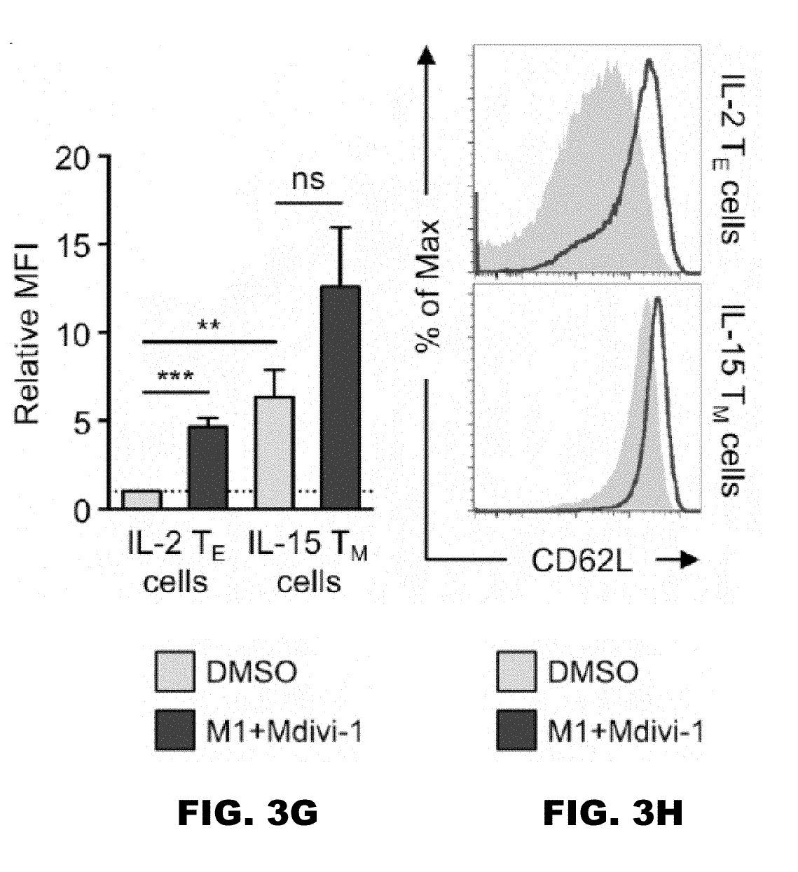

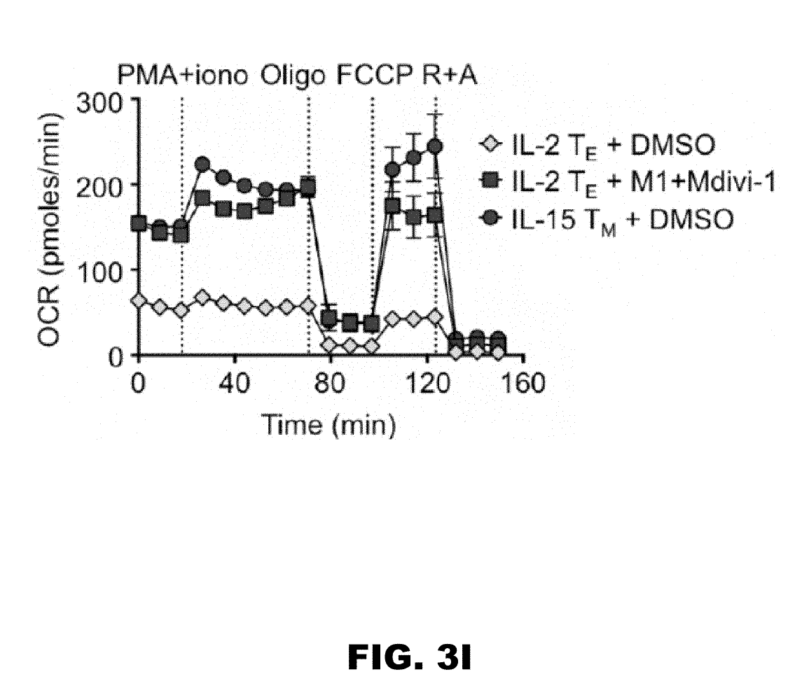

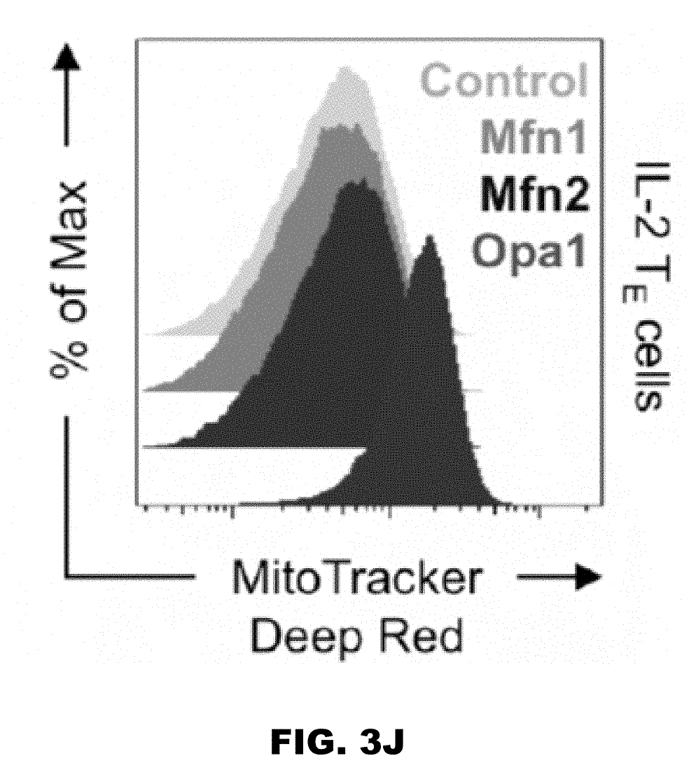

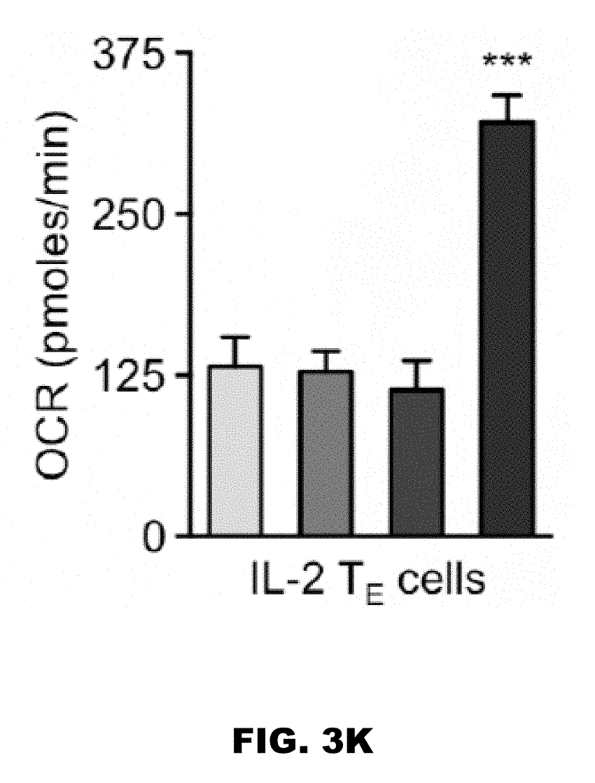

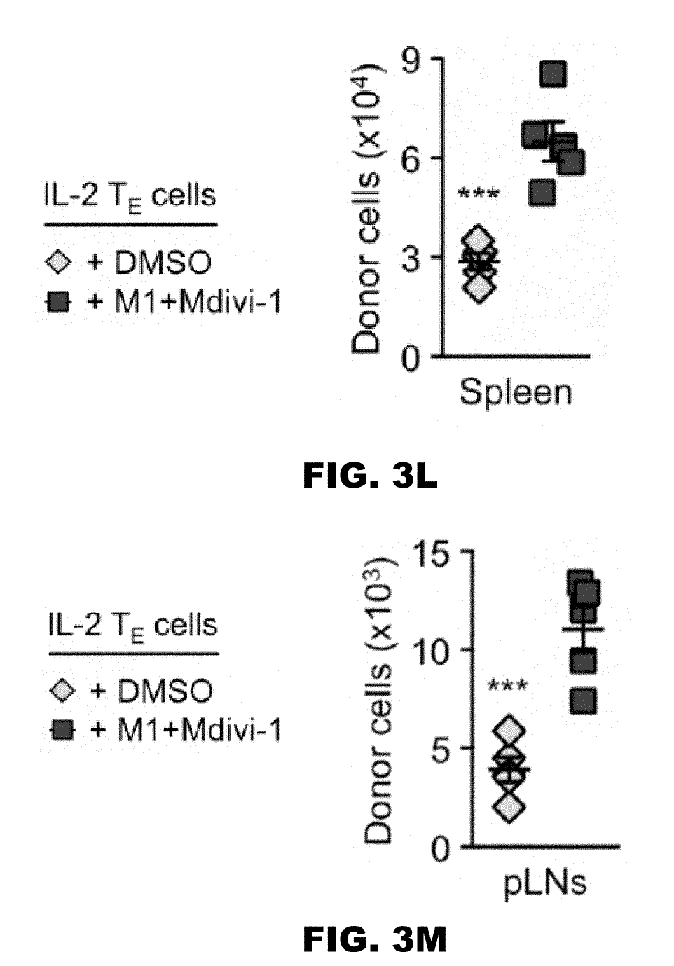

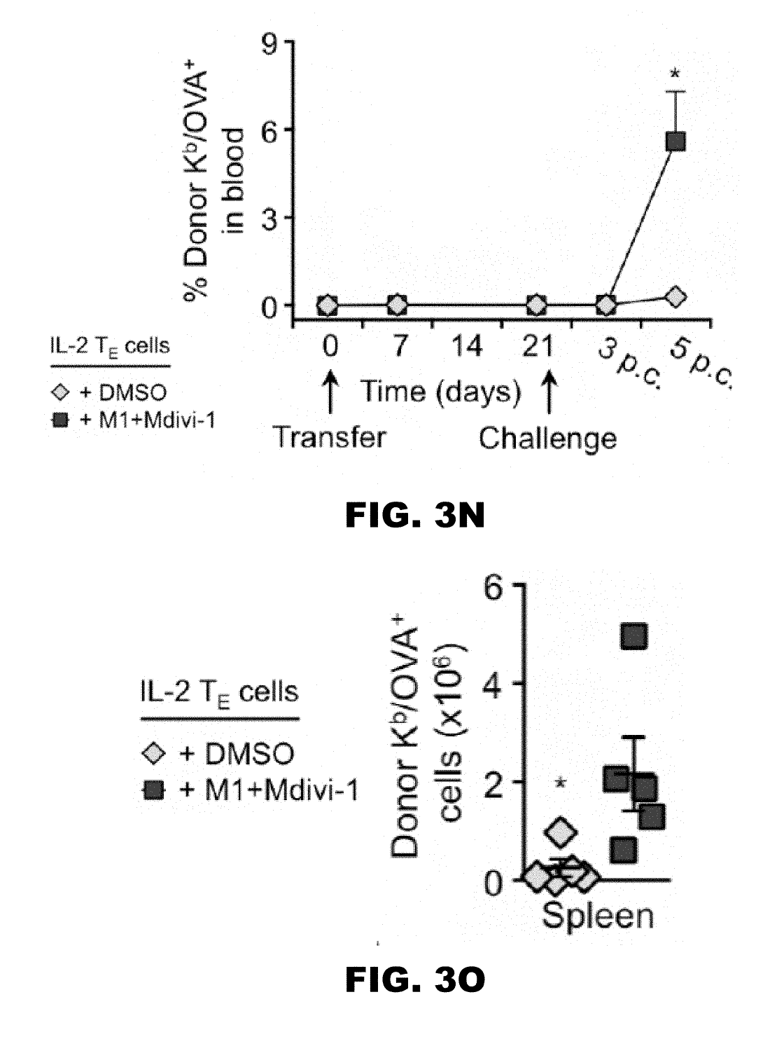

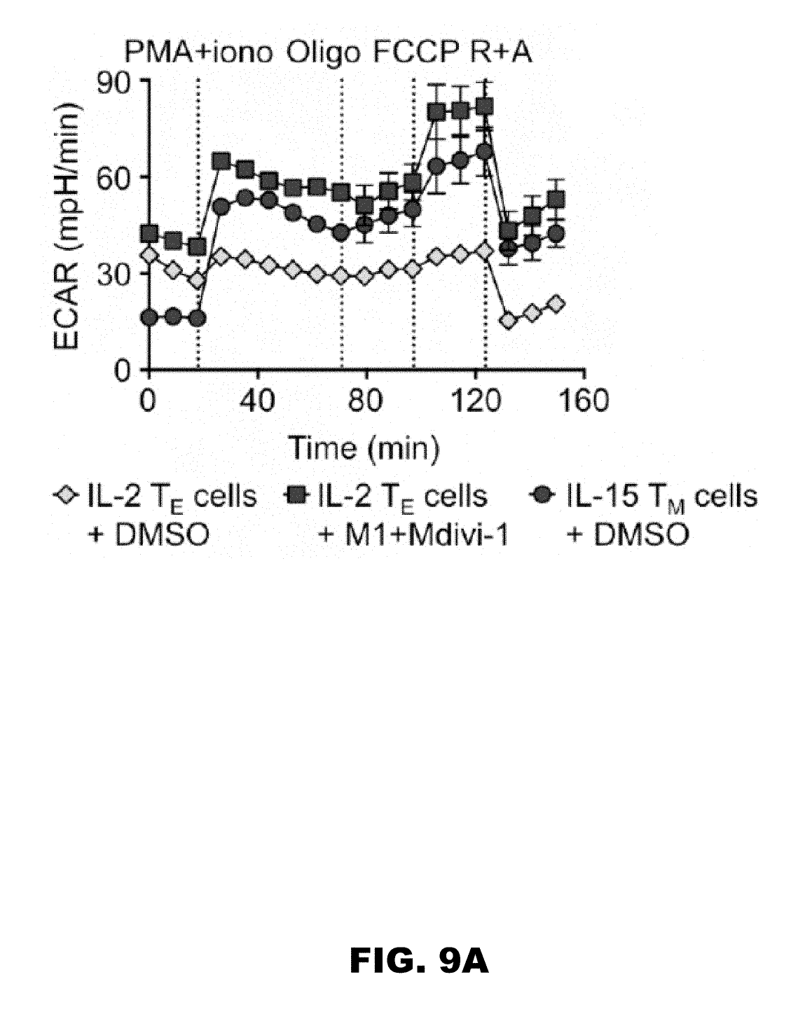

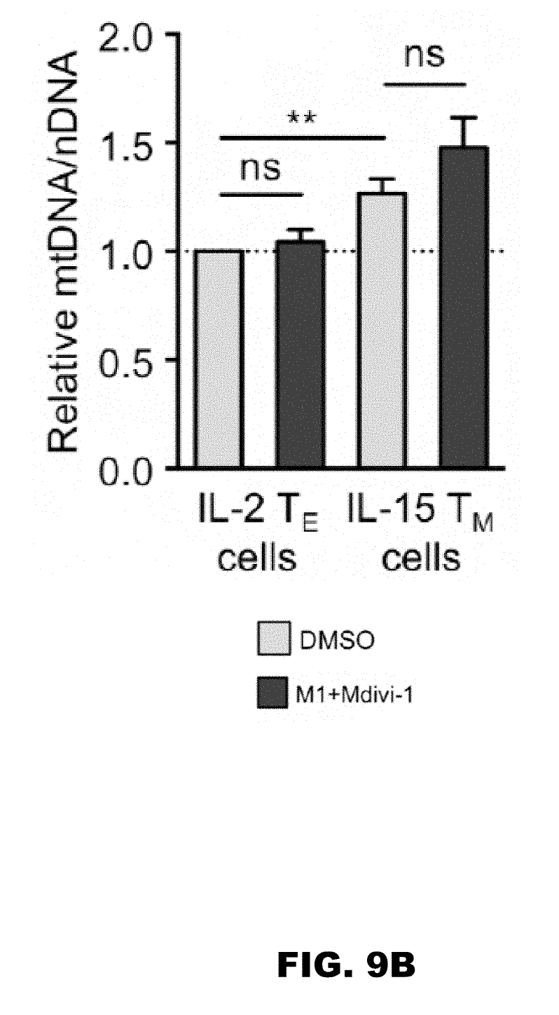

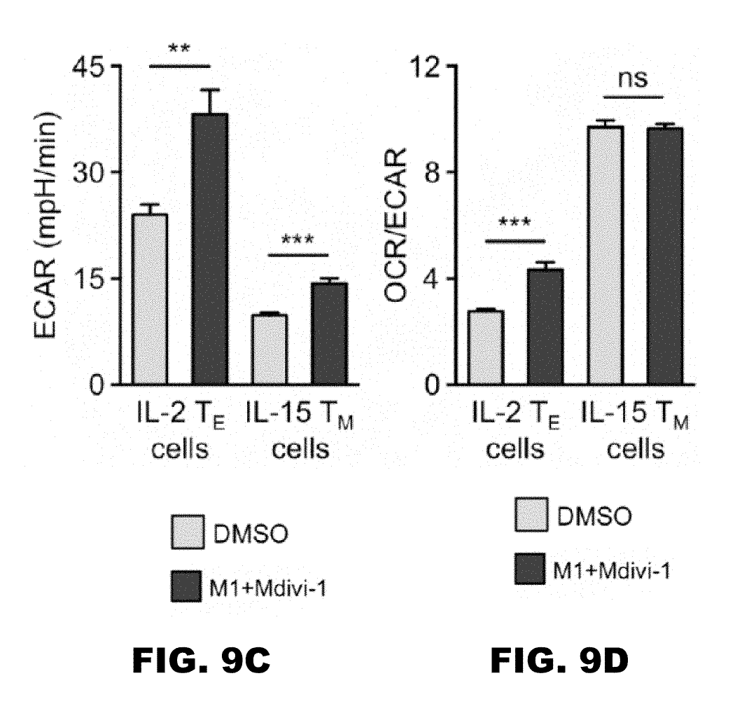

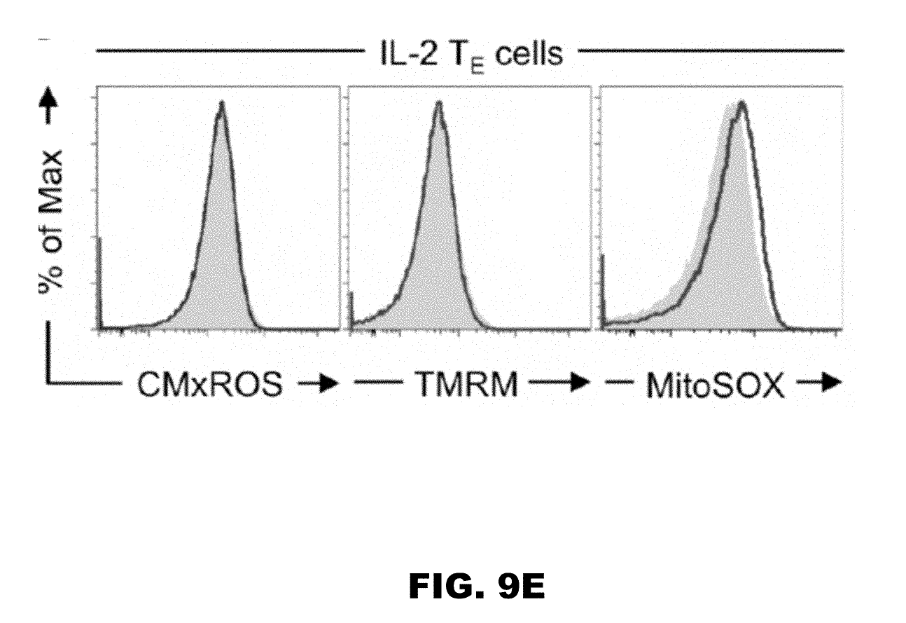

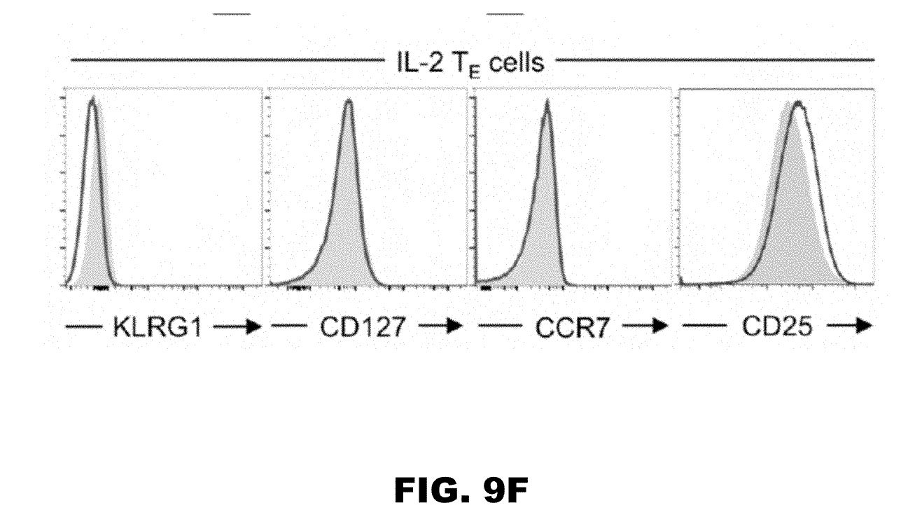

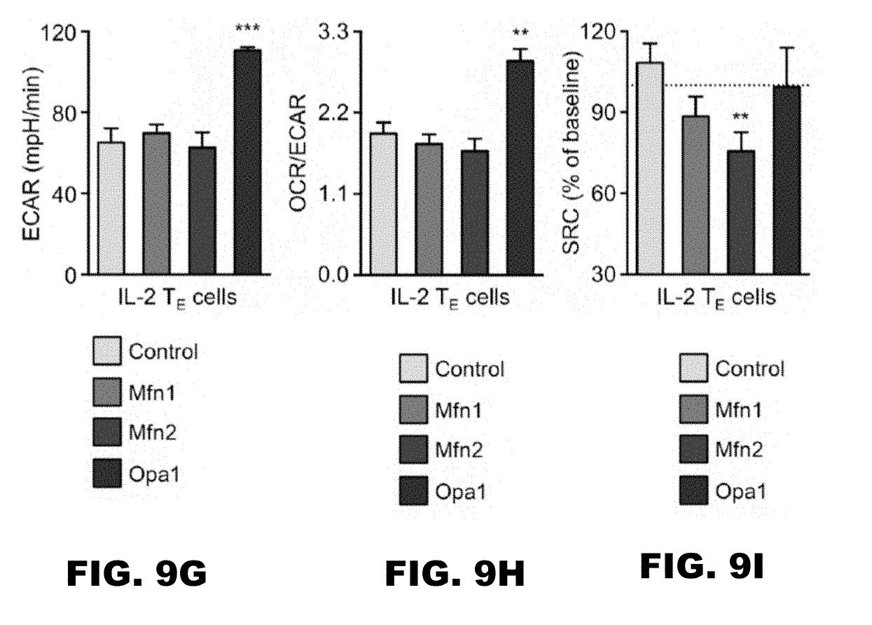

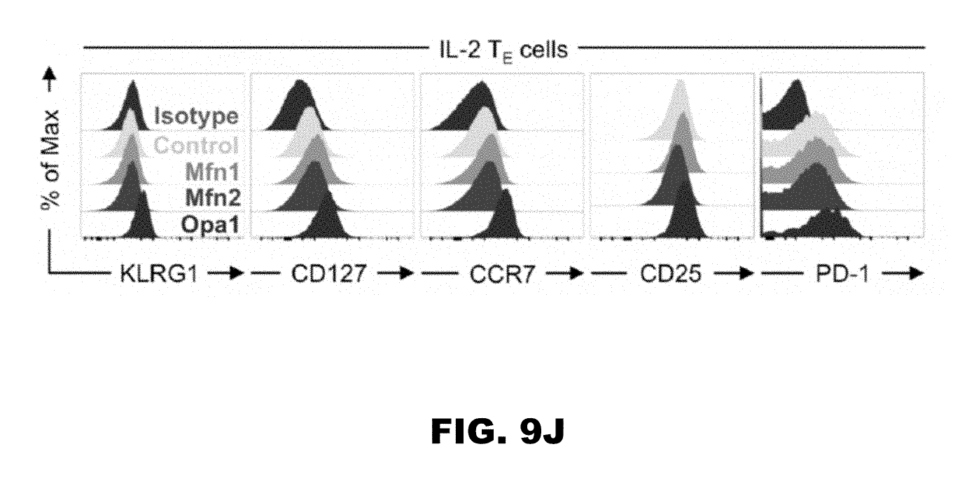

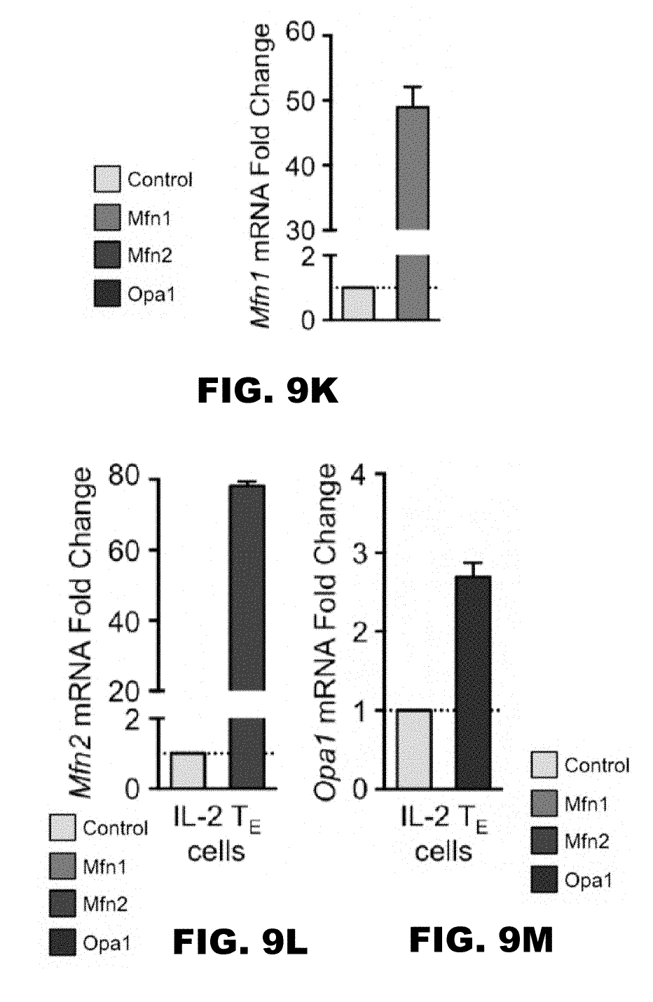

Genetic loss of function of Opa1 revealed that this protein is critical for T_{M} cell formation. Given the fused phenotype of mitochondria in these cells, we hypothesized that Opa1-mediated mitochondrial fusion supports the metabolism needed for T_{M} cell development. We used a gain of function approach to enhance mitochondrial fusion. Culturing T cells with the ‘fusion promoter’ M1, and the ‘fission inhibitor’ Mdivi-1 (FIG. 3A), induced mitochondrial fusion in IL-2 T_{E} cells, rendering them morphologically similar to IL-15 T_{M} cells (FIG. 3B). Treatment with these drugs enhanced other T_{M} cell properties in activated IL-2 T_{E} cells, including increased mitochondrial mass (FIG. 3C, FIG. 3D), OXPHOS and SRC (FIG. 3E, FIG. 3F), CD62L expression (FIG. 3G, FIG. 3H) and robust metabolic activity, as indicated by bioenergetic profiling of the cells in response to secondary stimulation with PMA/ionomycin, followed by addition of oligomycin (ATP synthase inhibitor), FCCP, and rotenone plus antimycin A (ETC complex I and III inhibitors), all drugs that stress the mitochondria (FIG. 3I and FIG. 9A) (Nicholls et al., 2010). However, we did not observe increased mtDNA in these cells (FIG. 9B). We found that ECAR and the OCR/ECAR ratio increased after drug treatment (FIG. 9C, FIG. 9D), indicating elevated metabolic activity overall, with a predominant increase in OXPHOS over glycolysis. While we observed these changes in mitochondrial activity, we did not measure any significant differences in mitochondrial membrane potential or ROS production after drug treatment (FIG. 9E). The expression of other activation markers were also not substantially affected, although a small decrease in KLRG1 and increase in CD25 was measured (FIG. 9F). Additionally, we performed a genetic gain of function experiment and transduced activated IL-2 T_{E} cells with retrovirus expressing Mfn1, Mfn2, or Opa1. Similar to enforcement of fusion pharmacologically, we found that cells transduced with Opa1 had more mitochondria (FIG. 3J) and OXPHOS (FIG. 3K), than empty vector control or Mfn-transduced T cells, as well as increased overall metabolic activity, with a predominant increase in OXPHOS over glycolysis (FIG. 9G, FIG. 9H, FIG. 9I). T_{M} cell associated markers such as CCR7 and CD127 were increased on transduced cells, as well as T_{E} cell proteins, such as PD-1 (FIG. 9J). We confirmed by mRNA expression that each target gene had increased expression after transduction over the control (FIG. 9J). Together our results show that mitochondrial fusion confers a T_{M} cell phenotype on activated T_{E} cells even in culture conditions that program T_{E} cell differentiation.

### Example 4. T Cell Mitochondrial Fusion Improves Adoptive Cellular Immunotherapy Against Tumors

A major consideration when designing adoptive cellular immunotherapy is to improve T cell fitness during ex vivo culture, so that when T cells are re-introduced into a patient they are able to function efficiently and persist for long periods of time (Restifo et al., 2012, Maus et al., 2014, O'Sullivan and Pearce, 2015). Our data showed that fusion-promoting drugs created metabolically fit T cells. We hypothesized that enforced fusion would also enhance the longevity of IL-2 T_{E} cells in vivo. To test this, we adoptively transferred control and M1+Mdivi-1 treated OT-I T cells into congenic mice and tracked donor cell survival. We found significantly more drug treated T cells in the spleen (FIG. 3L) and lymph nodes (FIG. 3M) 2 days after transfer. To determine if the persistence of these cells would be maintained better long term than control cells, we infected mice with LmOVA more than 3 weeks later and measured T cell responses against the bacteria. We found that drug-treated cells selectively expanded in response to infection (FIG. 3N) and could be recovered in significantly greater numbers in the spleen 6 days post-challenge (FIG. 3O).

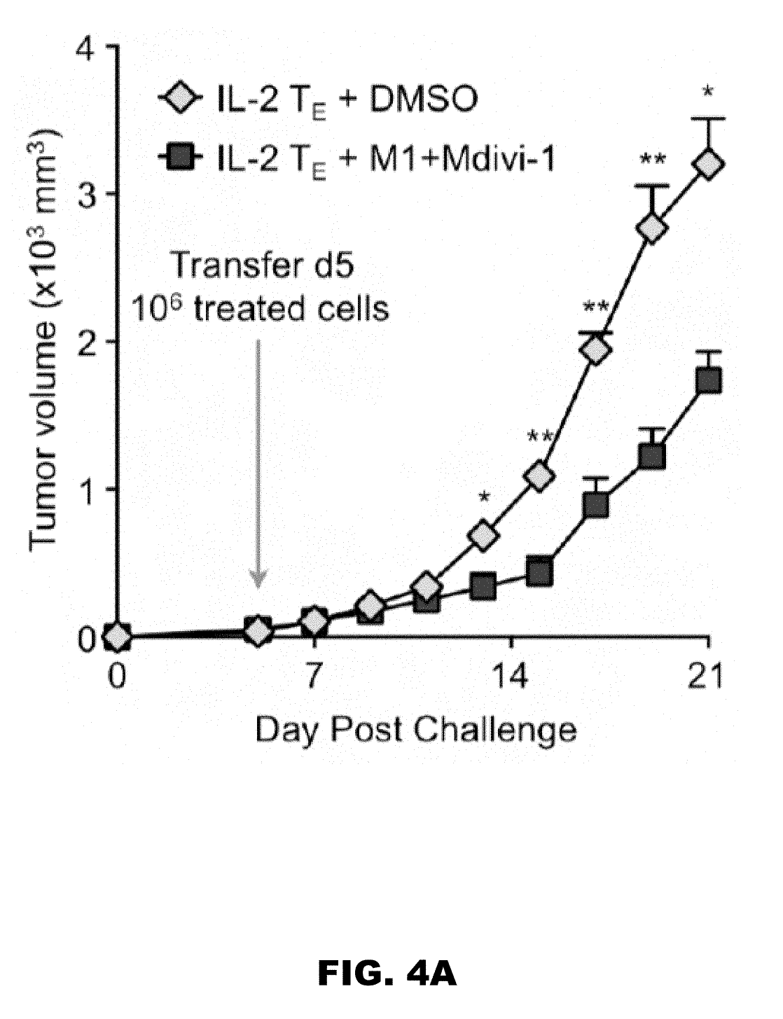

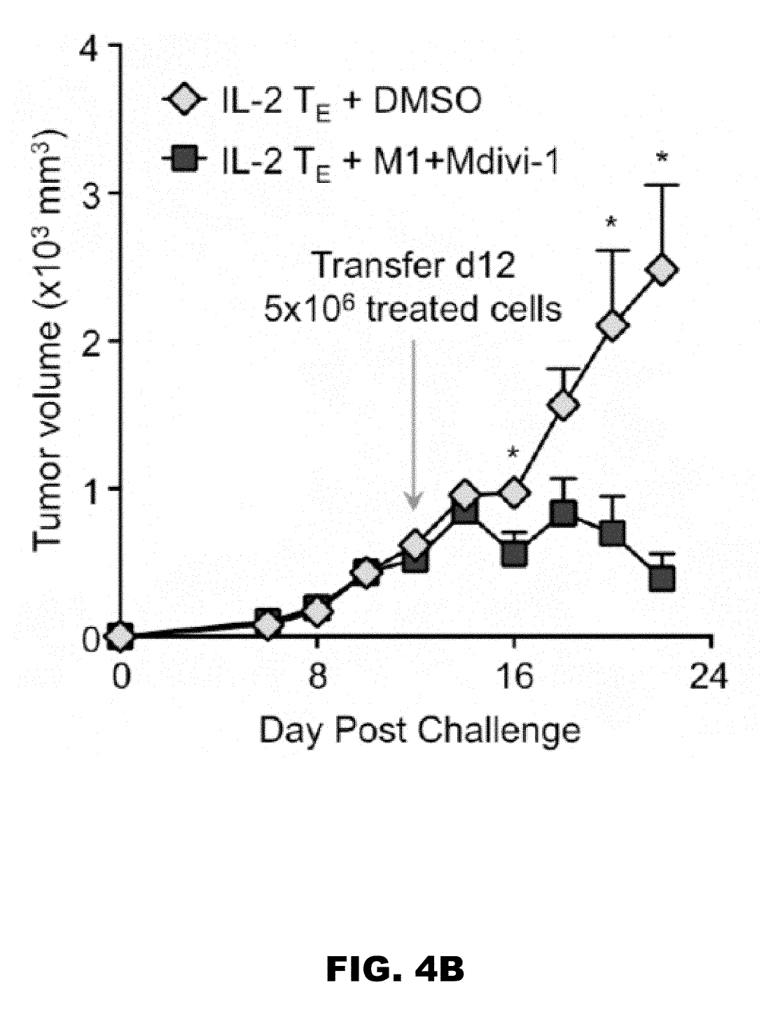

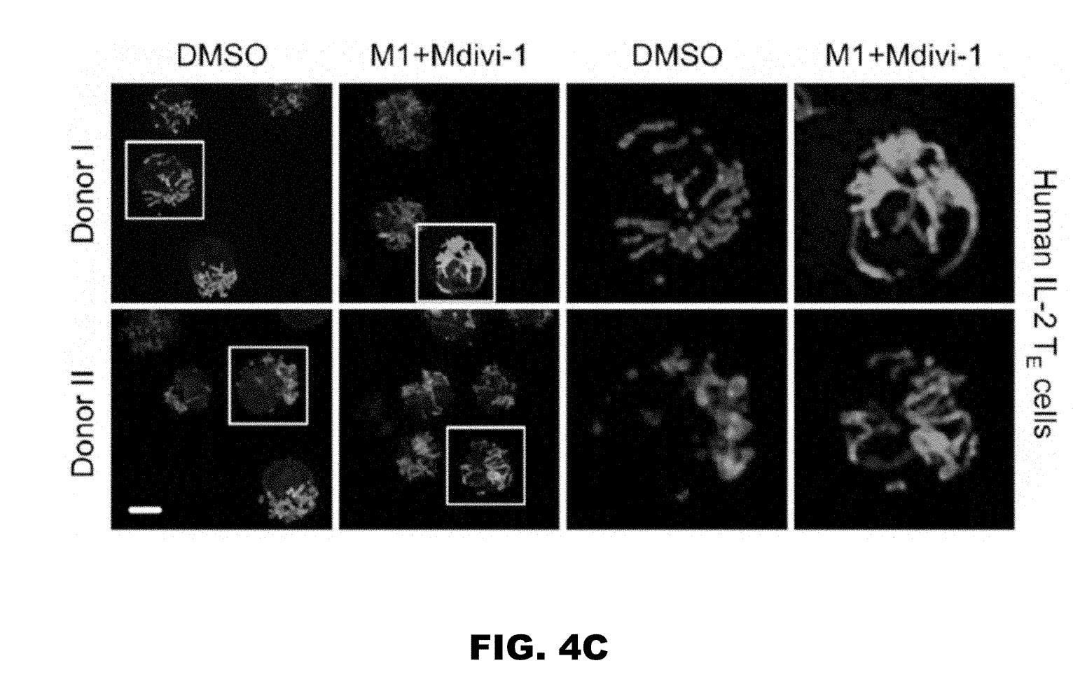

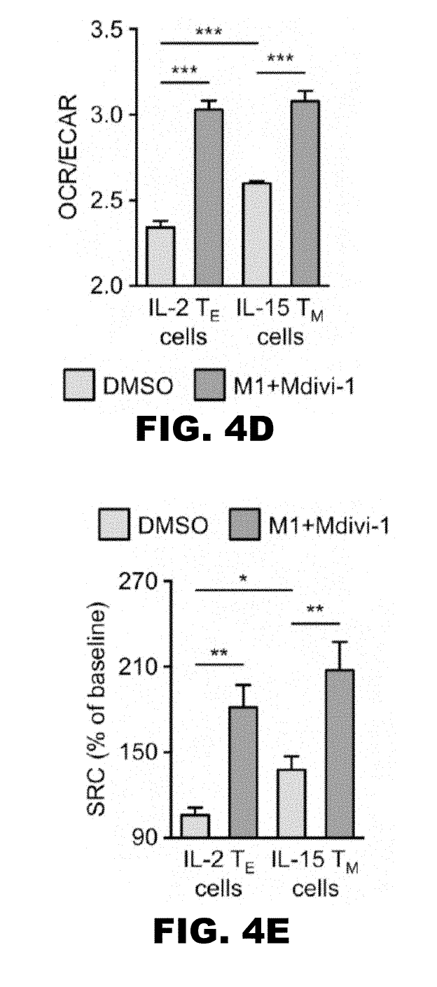

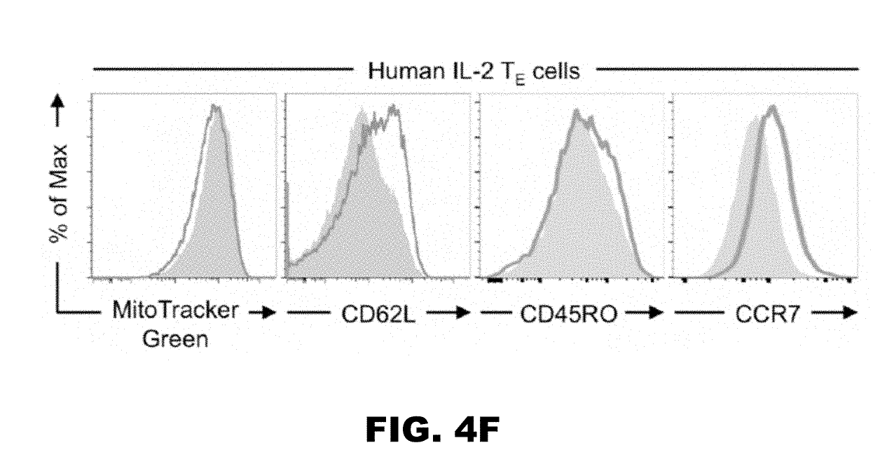

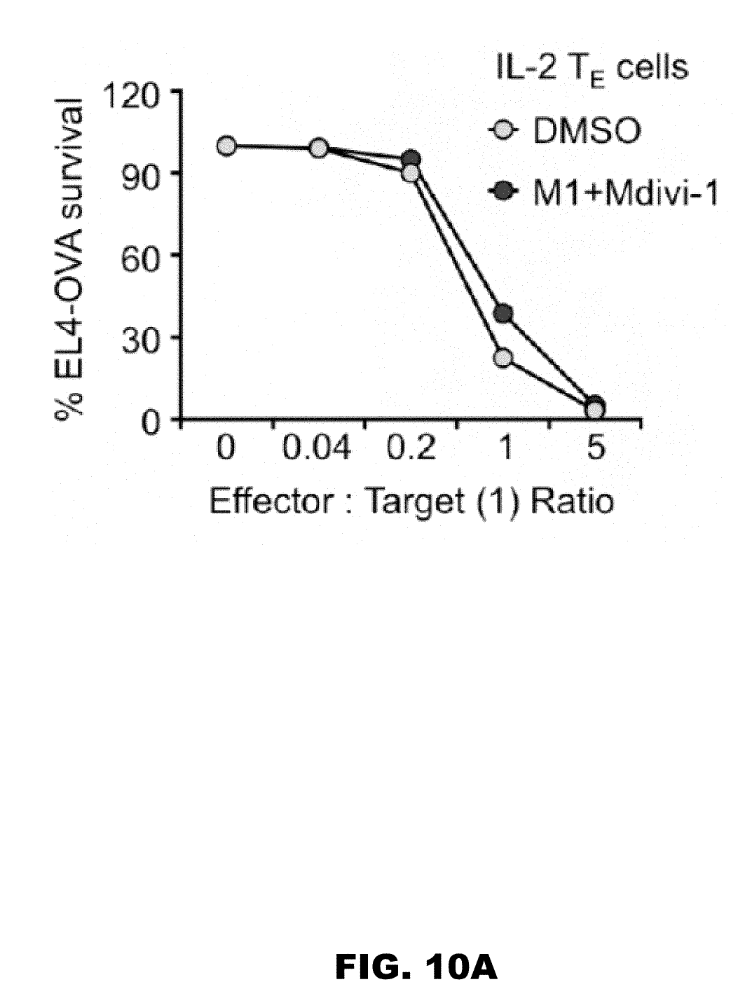

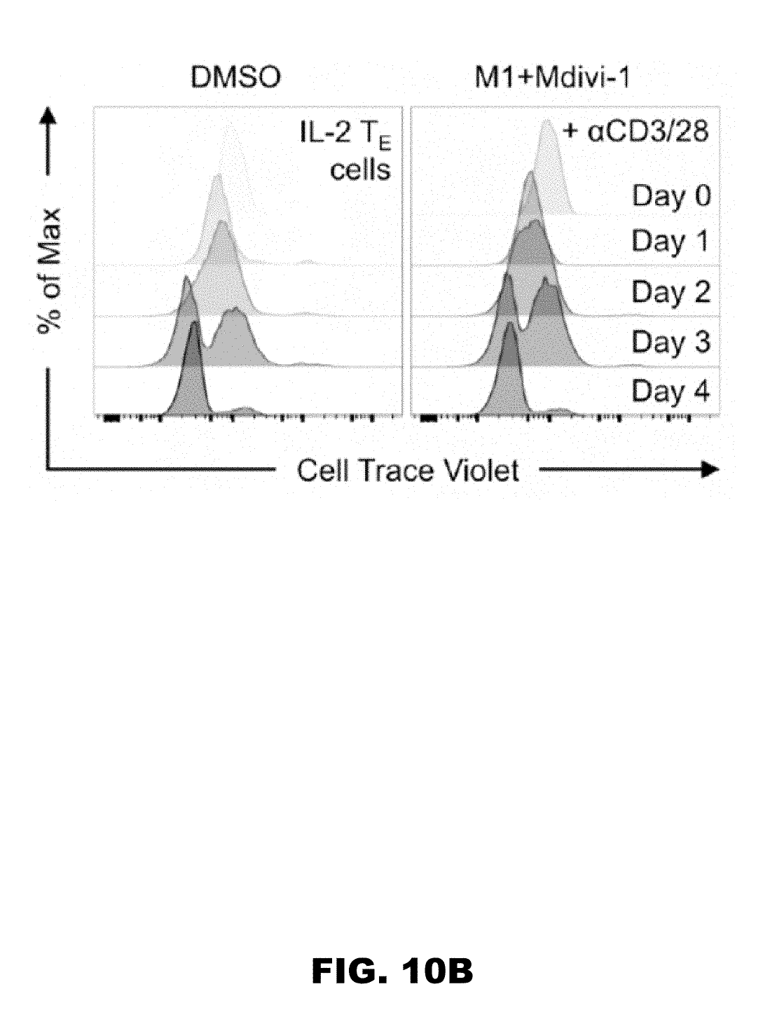

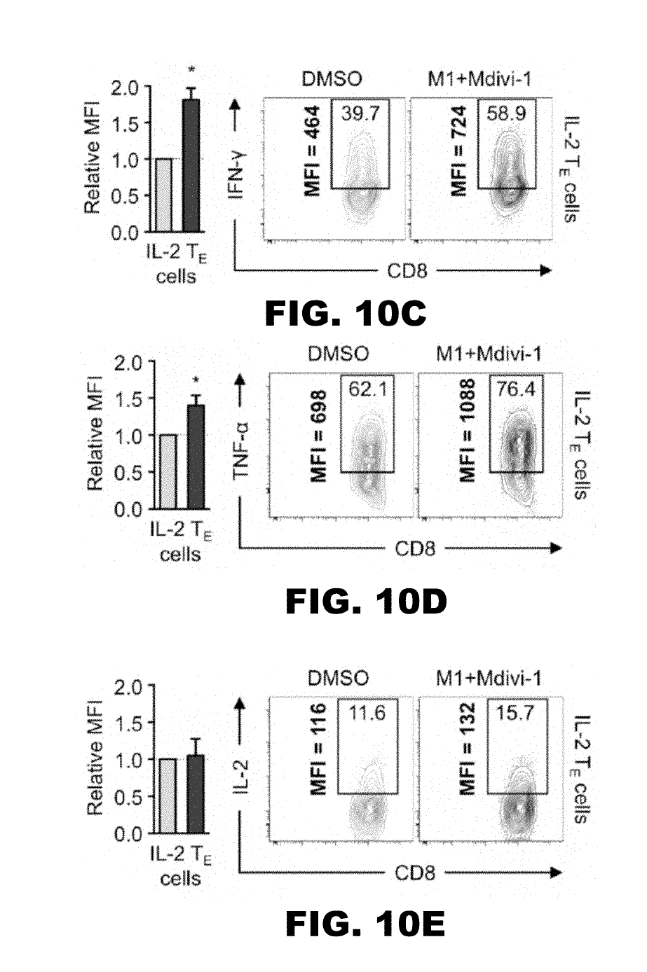

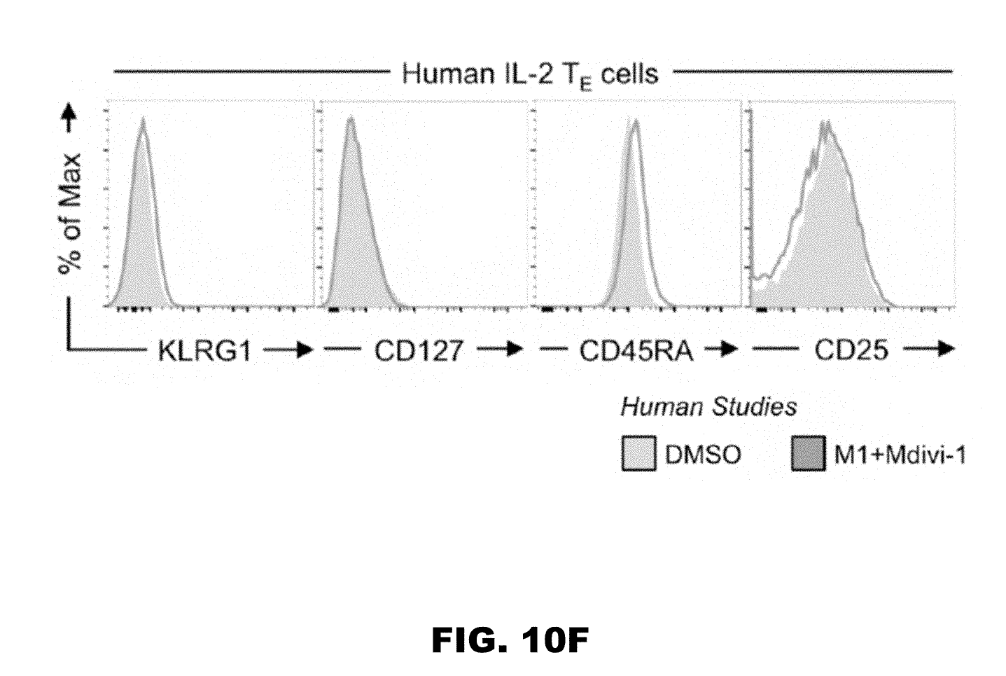

Next, we assessed whether these drugs could be used to promote T cell function in a model of adoptive cell immunotherapy. We injected EL4-OVA tumor cells into mice. Then either 5 or 12 days later we adoptively transferred IL-2 T_{E} cells that had been previously treated with DMSO or M1+Mdivi-1. In both settings, mice that had received ‘fusion-promoted’ T cells were able to control tumor growth significantly better than mice that had received control treated cells (FIG. 4A, FIG. 4B). The cytolytic ability (FIG. 10A) and proliferation (FIG. 10B) of the modified IL-2 T_{E} cells were similar to control cells, however, fusion enforced IL-2 T_{E} cells expressed significantly higher levels of IFN-γ and TNF-α when restimulated with PMA and ionomycin in vitro (FIG. 10C). We also exposed activated human T cells to M1+Mdivi-1 treatment in vitro and found that activated human IL-2 T_{E} cells had visibly more fused mitochondria (FIG. 4C), and exhibit the bioenergetic profile (FIG. 4D, FIG. 4E), and surface marker expression (FIG. 4F) characteristic of T_{M} cells, compared to control treated cells. Parameters such as mitochondrial mass (FIG. 4F) and other surface markers (FIG. 10D) were not significantly altered. These data suggest that promoting fusion in T cells may be translatable treatment for enhancing human therapy.

### Example 5. Mitochondrial Fusion Promotes T_{M} Cell Metabolism, but Opa1 is not Required for FAO

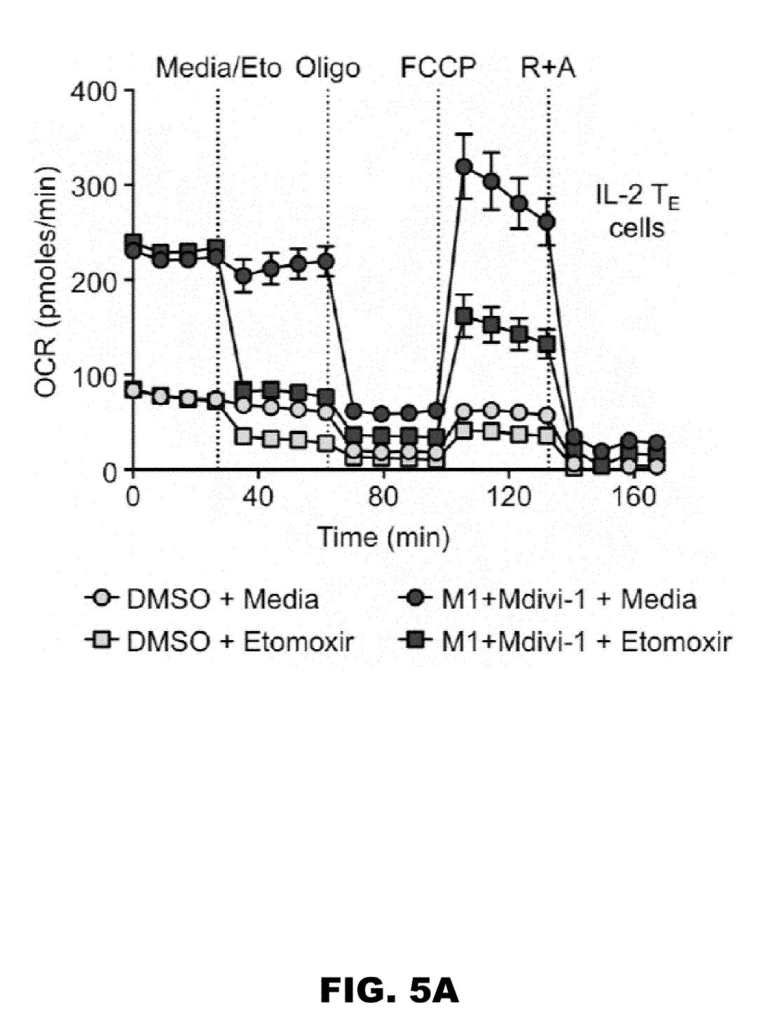

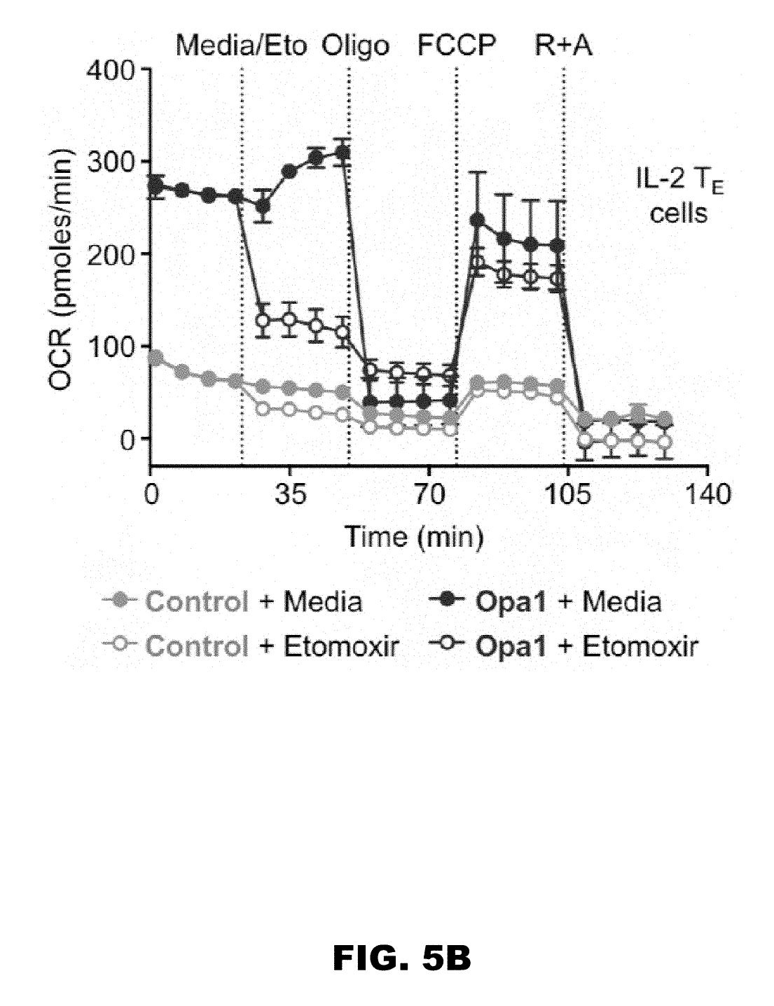

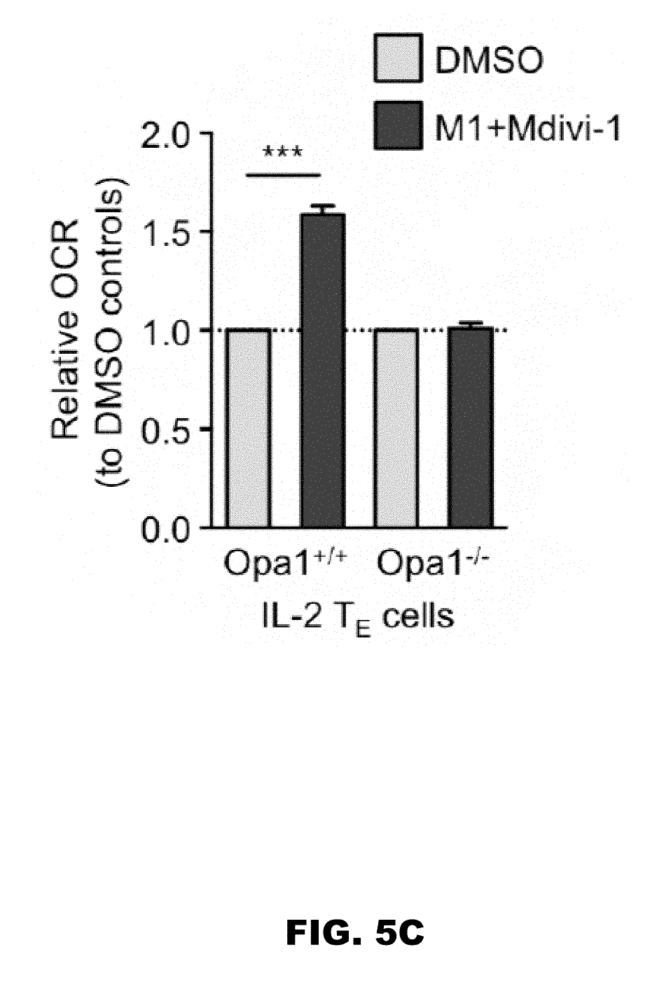

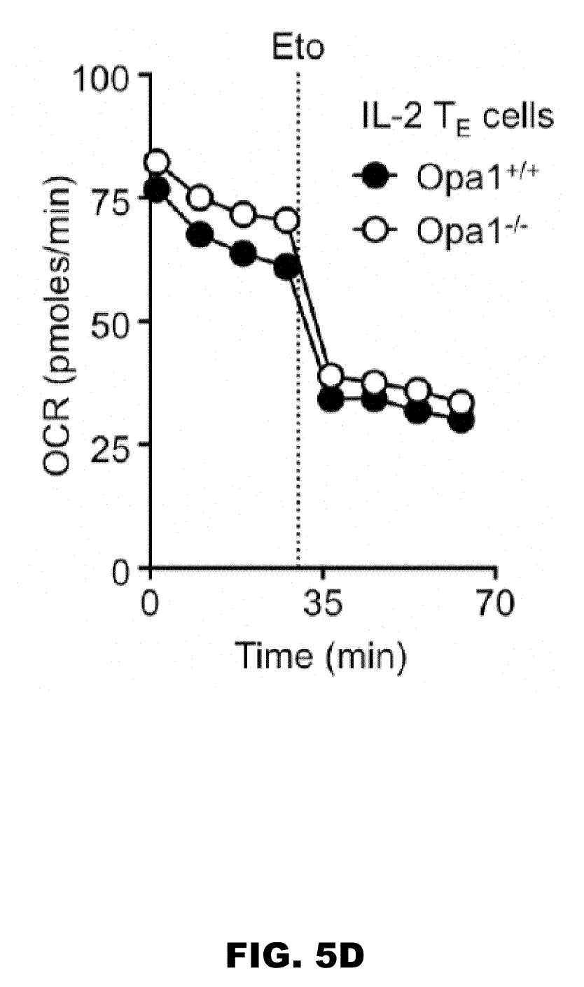

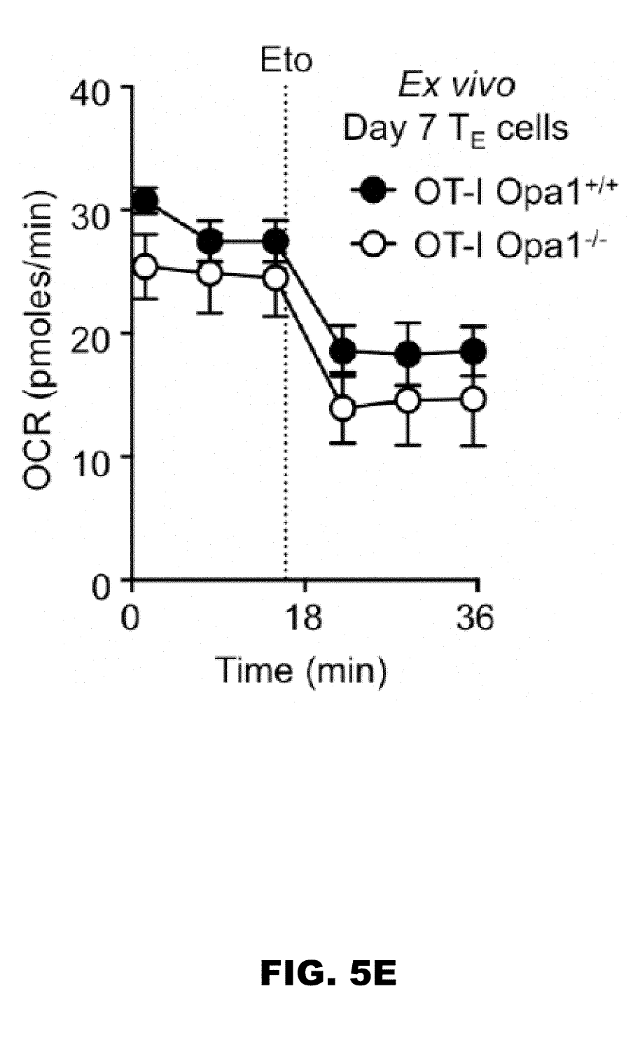

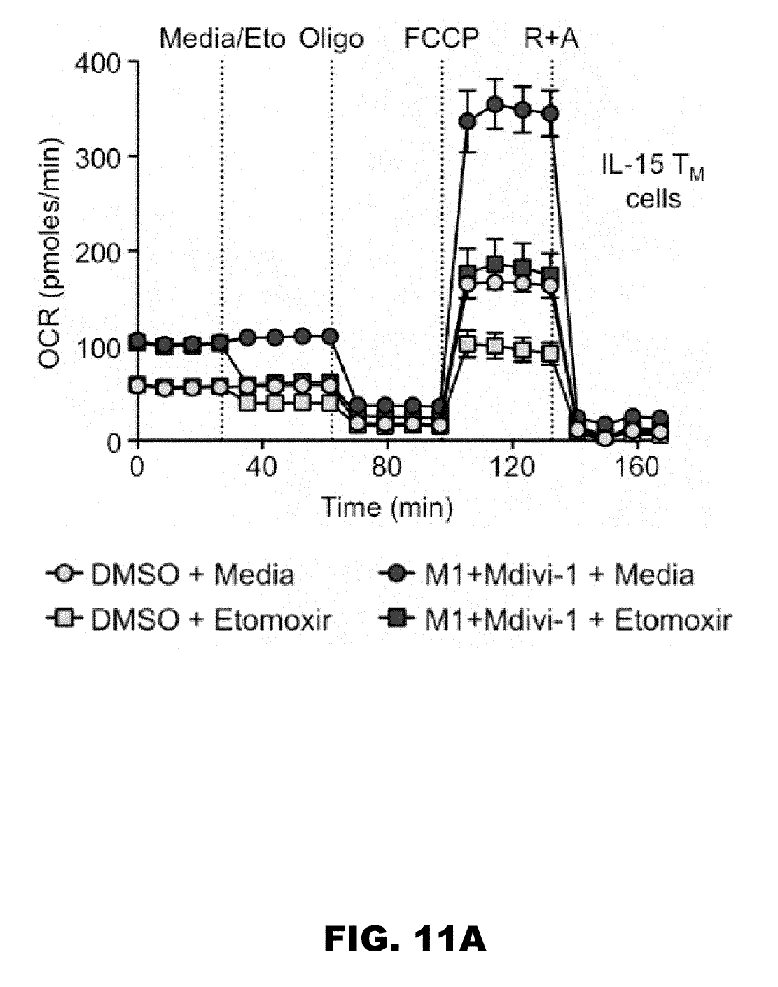

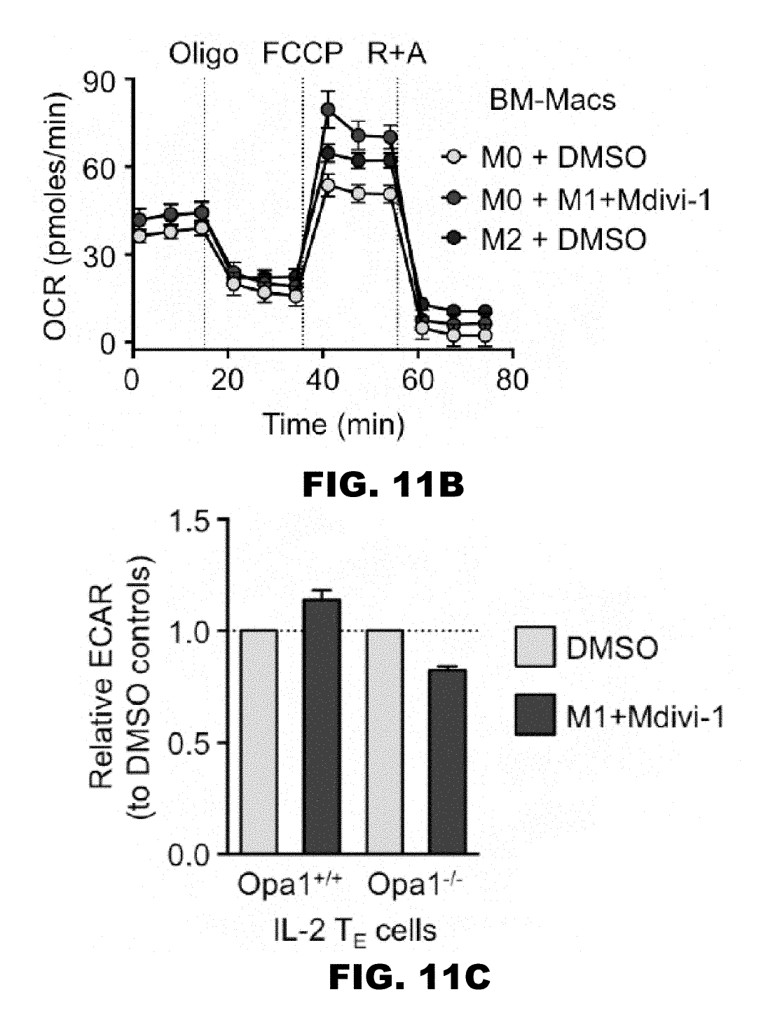

Our data showed that Opa1 was a necessary regulator of T_{M} cell development, but the question of precisely how Opa1 acted to support T_{M} cells remained. We hypothesized that mitochondrial fusion, via Opa1 function, was needed for FAO, as the engagement of this pathway is a requirement for T_{M} cell development and survival (Pearce et al., 2009, van der Windt et al., 2012, van der Windt et al., 2013). This hypothesis was not only based on our observations that these two processes seemed to be linked in T_{M} cells, but also knowledge that mitochondrial fusion is important for efficient FAO via lipid droplet trafficking under starvation conditions. We treated IL-2 T_{E} and IL-15 T_{M} cells with M1+Mdivi-1 or vehicle and then measured OCR in response to etomoxir, a specific inhibitor of mitochondrial long chain FAO (Deberardinis et al., 2006), and mitochondrial inhibitors. We found that the increased OCR and SRC evident in these cells after M1+Mdivi-1 treatment was due to augmented FAO (FIG. 5A and FIG. 11A). IL-2 T_{E} cells transduced with Opa1 also exhibited enhanced OCR that decreased in the presence of etomoxir compared to control cells (FIG. 5B). Bone marrow derived macrophages (BM-Macs) cultured with M1+Mdivi-1 also increased OCR and SRC to levels similar as M2 polarized macrophages, which engage FAO much like T_{M} cells do (FIG. 11B) (Huang et al., 2014). Importantly, M1+Mdivi-1 treatment did not increase OCR (FIG. 5C) and did not affect ECAR (FIG. 11C) in Opa1^{-/-} IL-2 T_{E} cells compared to controls, suggesting a requirement for Opa1 in augmenting OCR and FAO. However, in contrast to what we expected, when we assessed bioenergetics of Opa1^{+/+} and Opa1^{-/-} IL-2 T_{E} cells (FIG. 5D) and ex vivo isolated T_{E} cells (FIG. 5E), we found that both cell types are equally responsive to etomoxir. Our results show that while Opa1 could promote FAO in T cells, it was not compulsory for engagement of this metabolic pathway.

### Example 6. Mitochondrial Cristae Remodeling Signals Metabolic Adaptations in T_{M} and T_{E} Cells

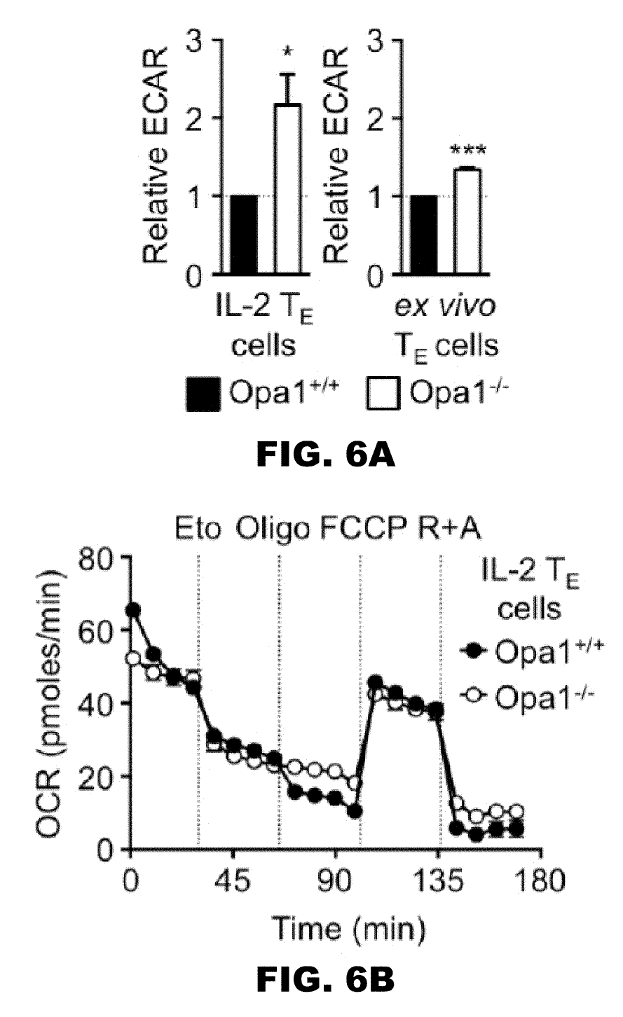

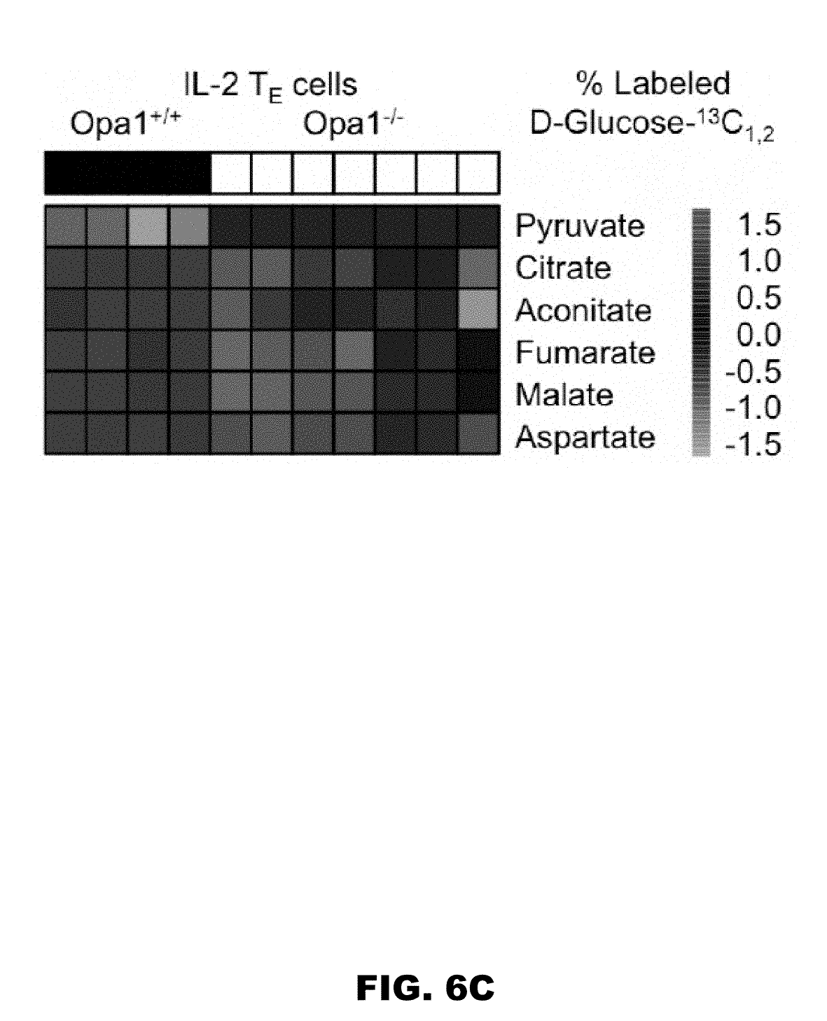

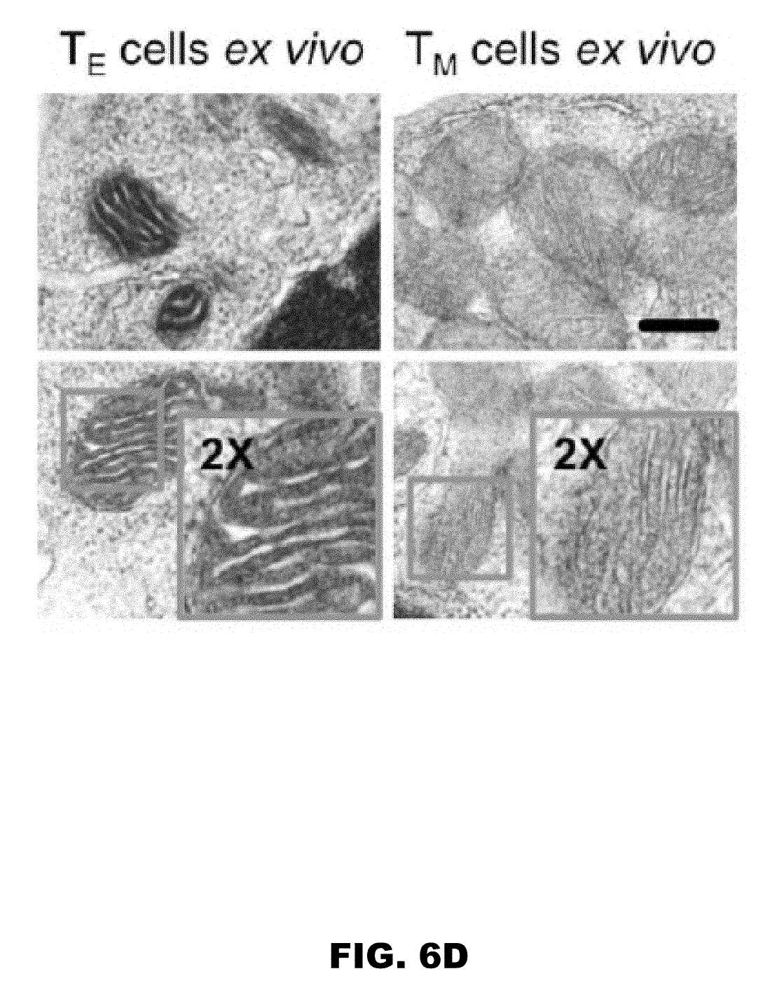

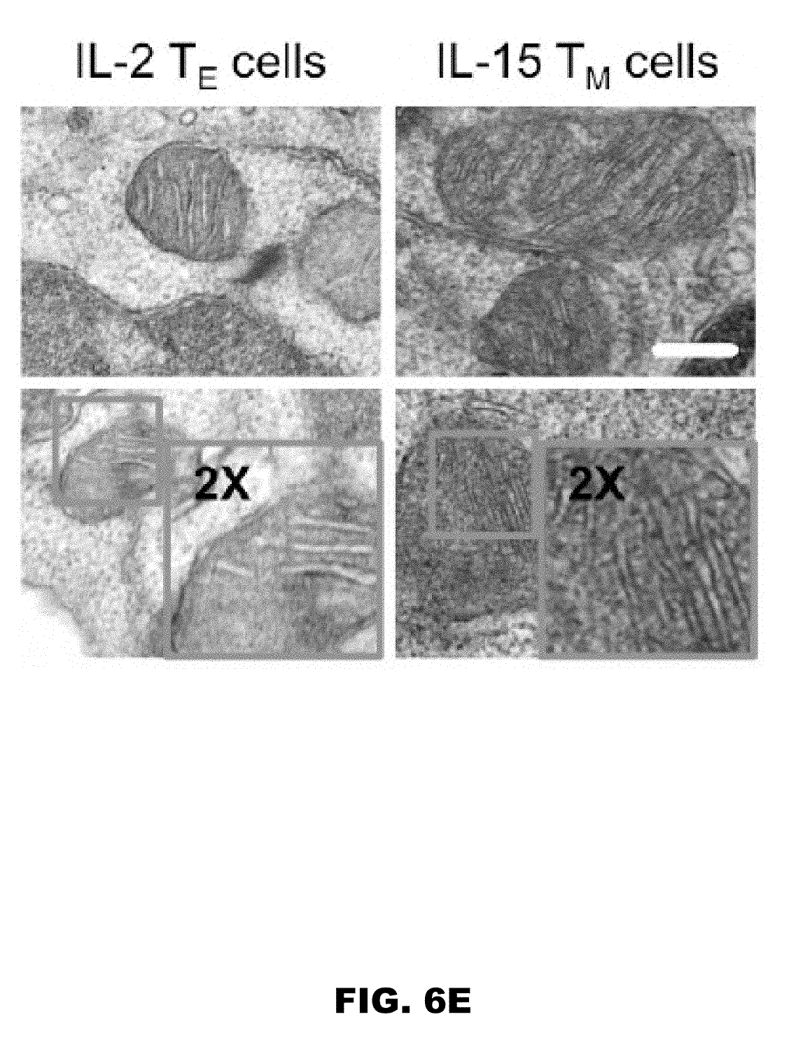

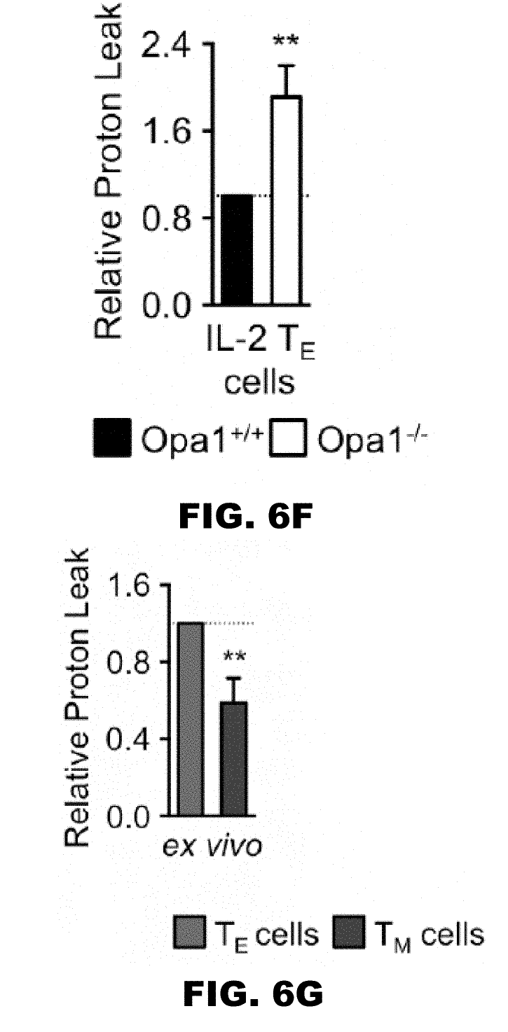

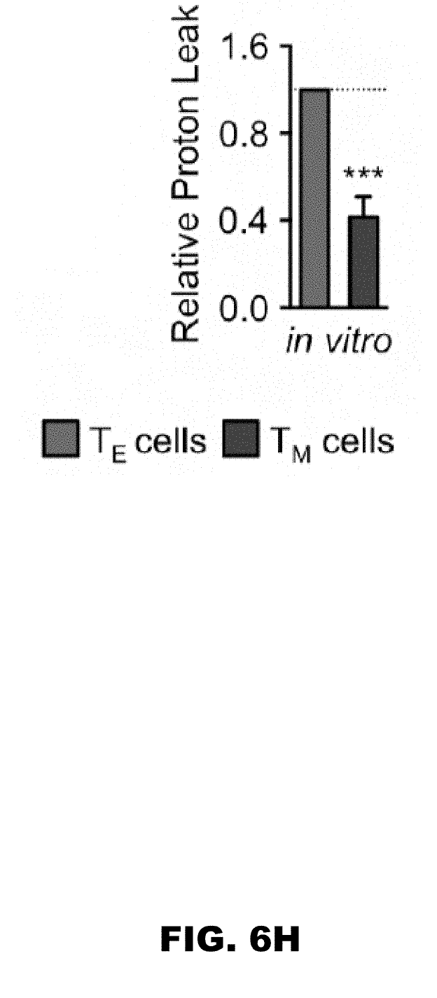

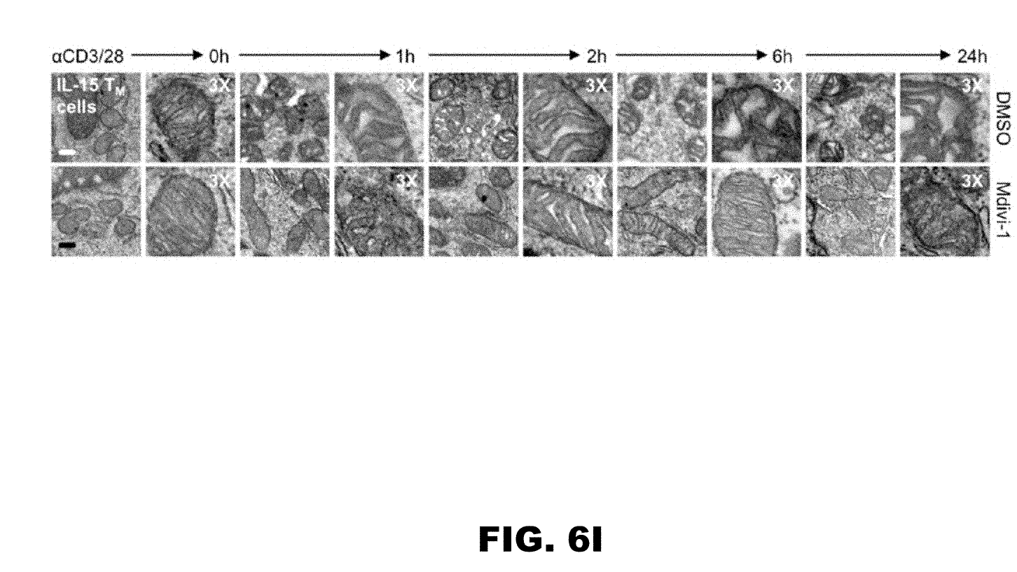

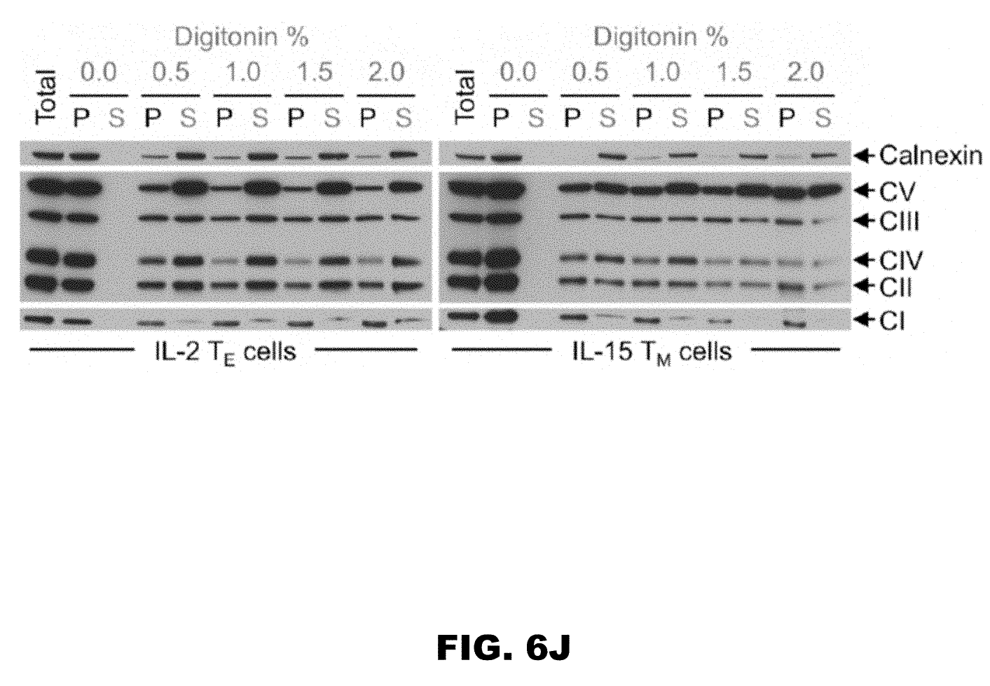

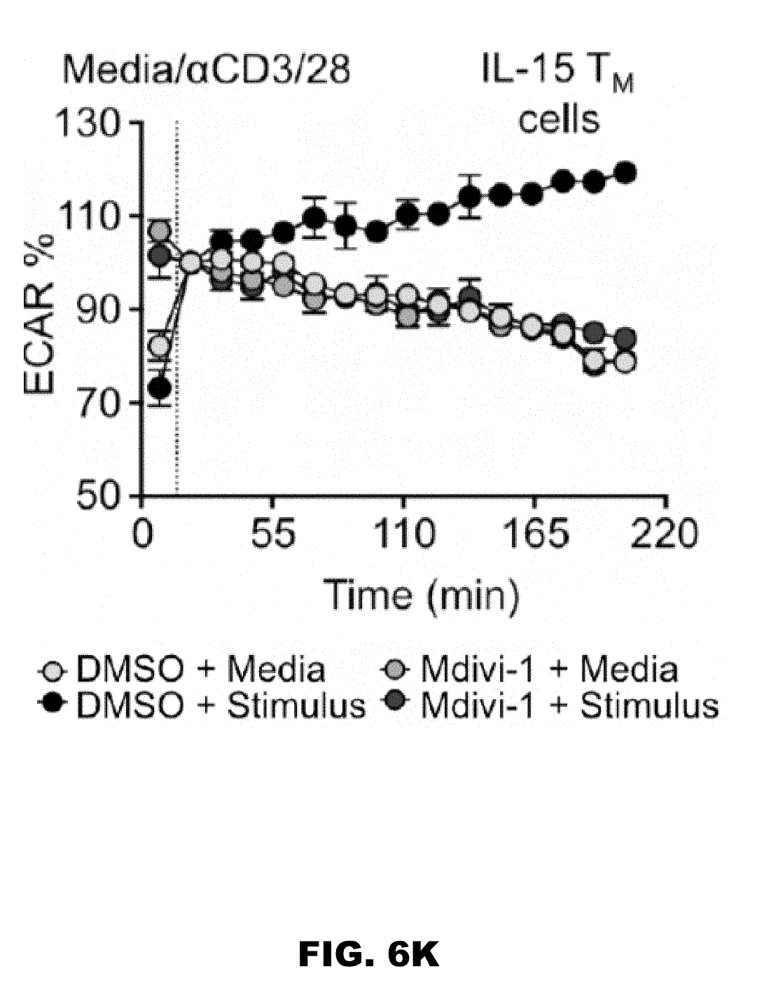

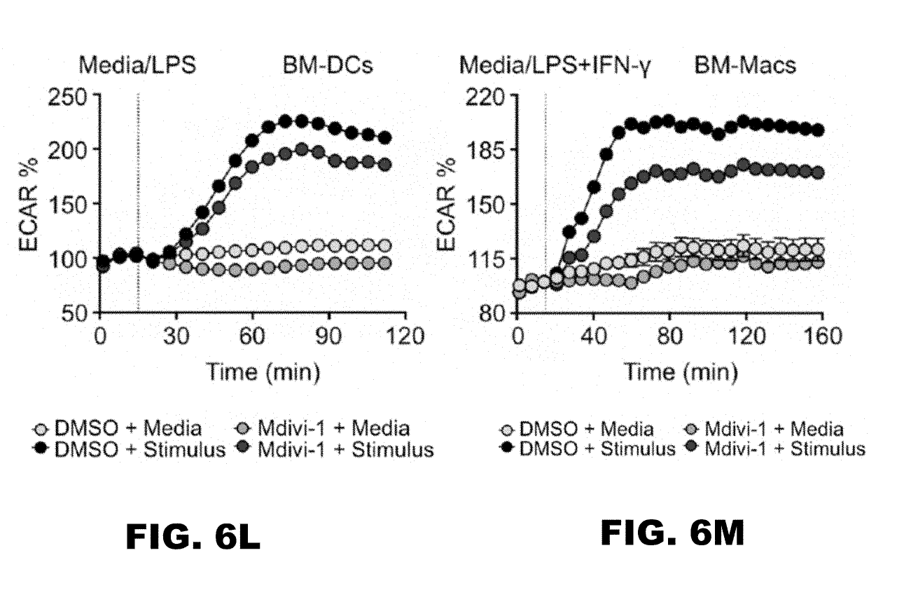

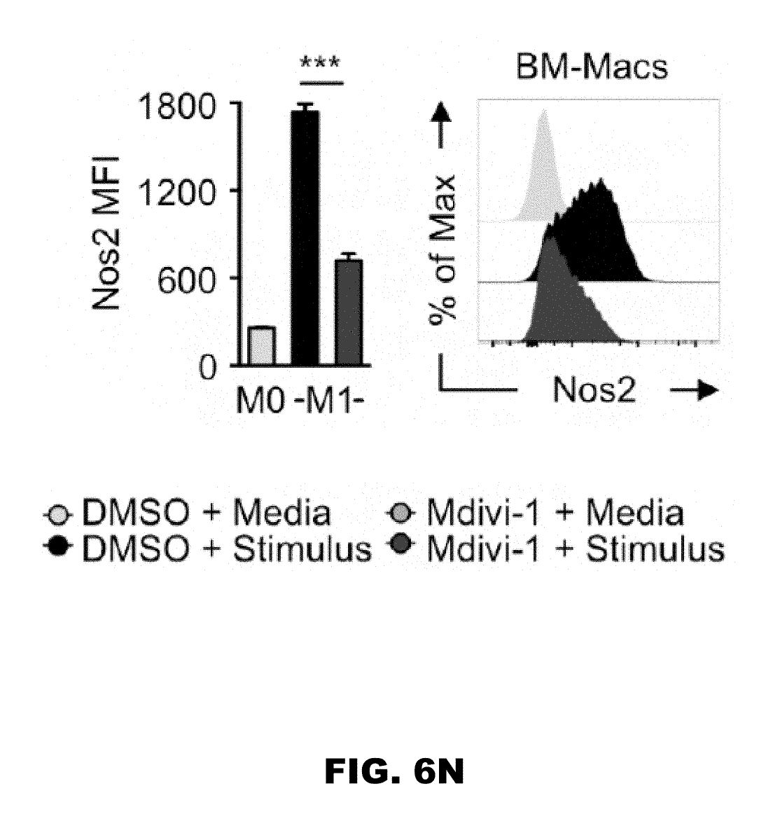

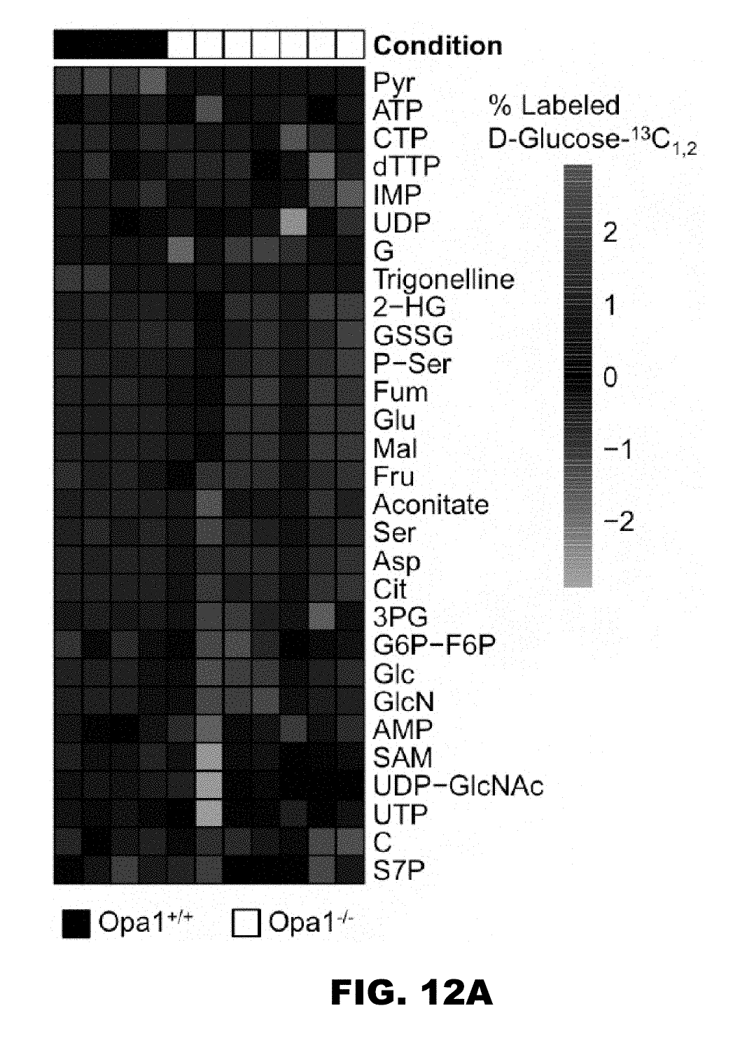

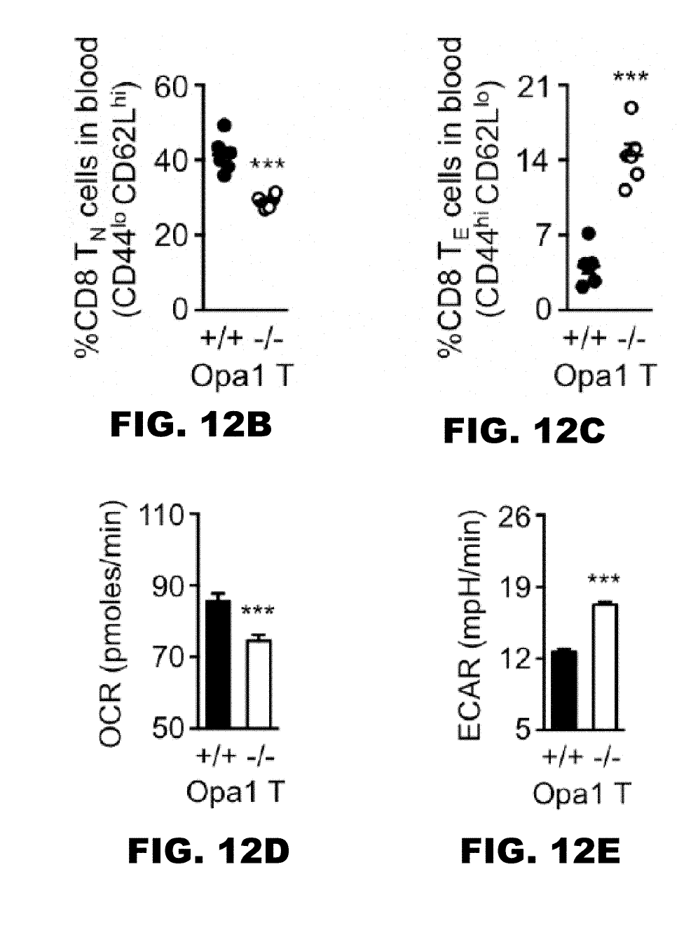

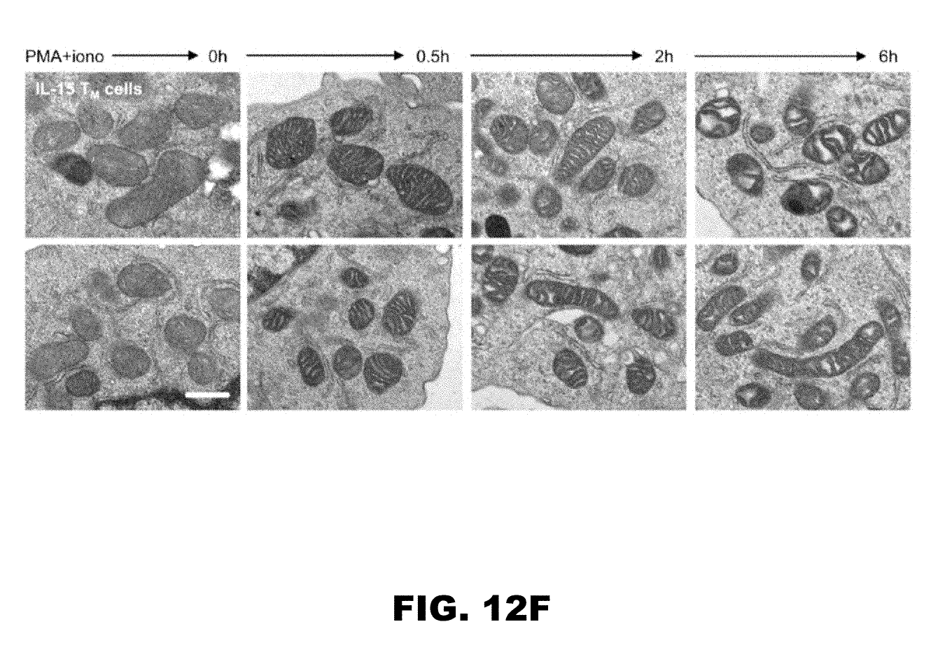

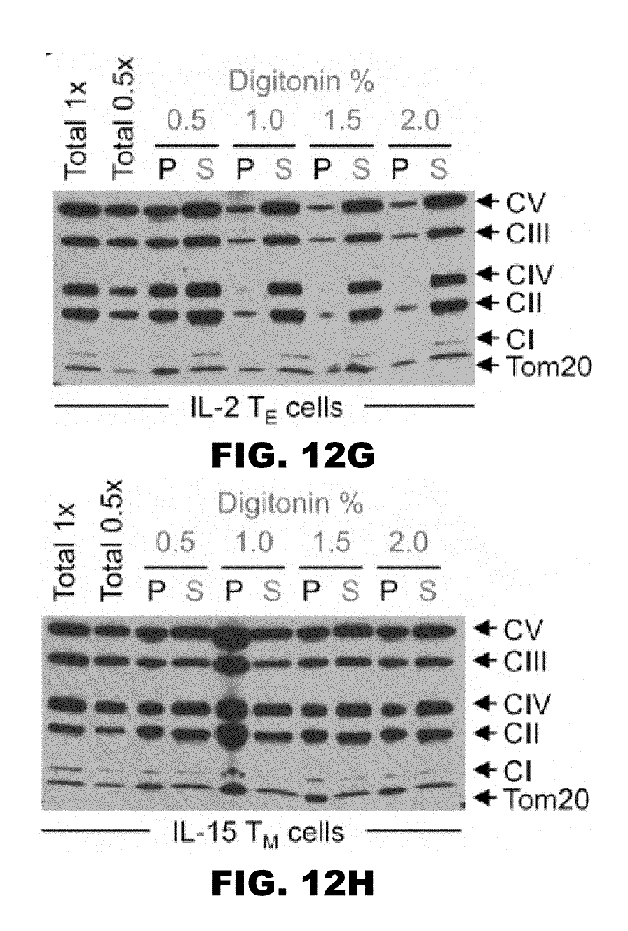

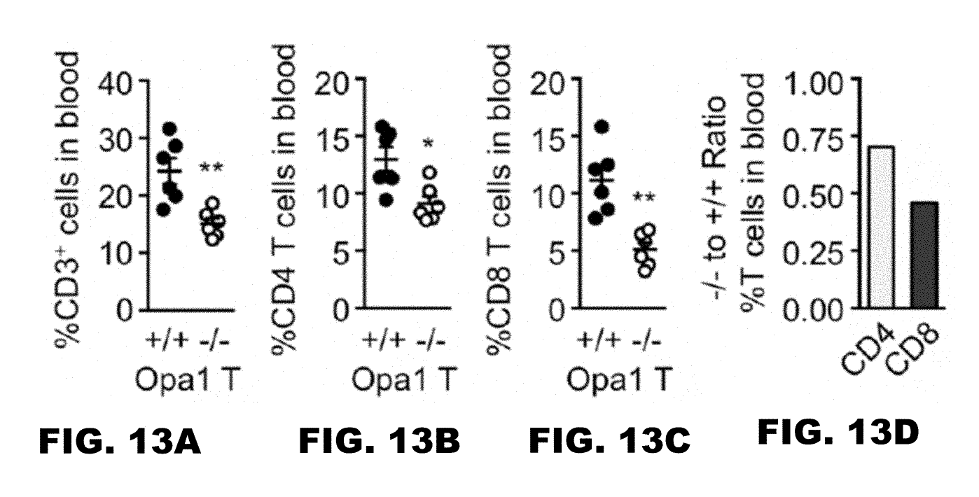

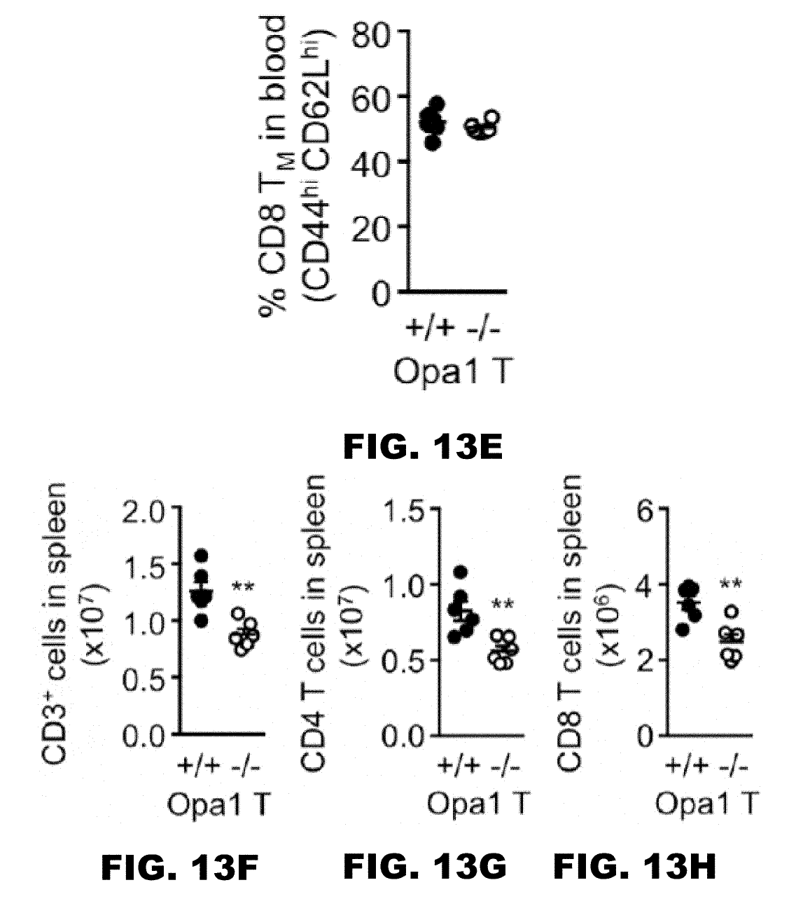

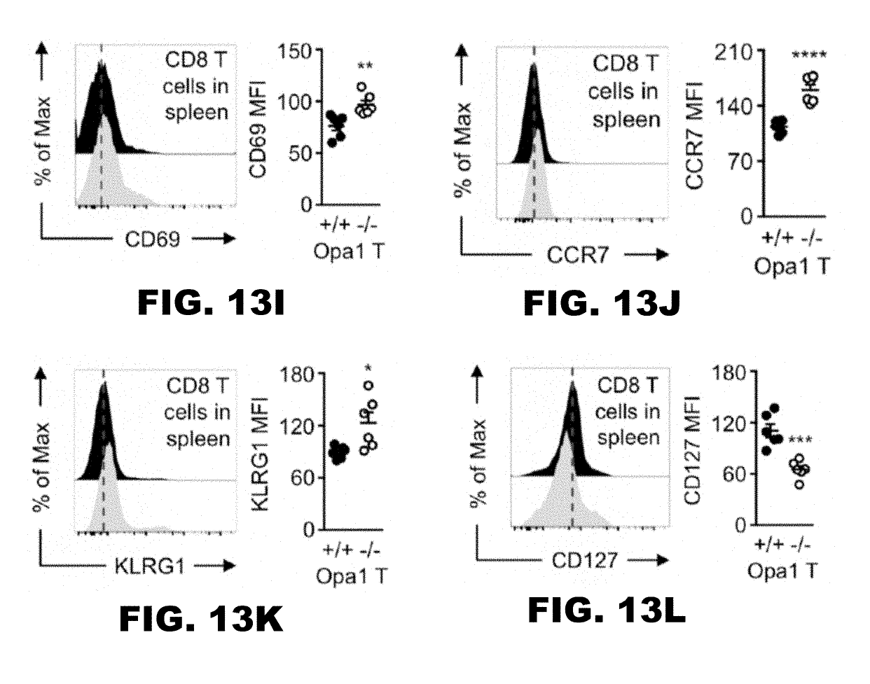

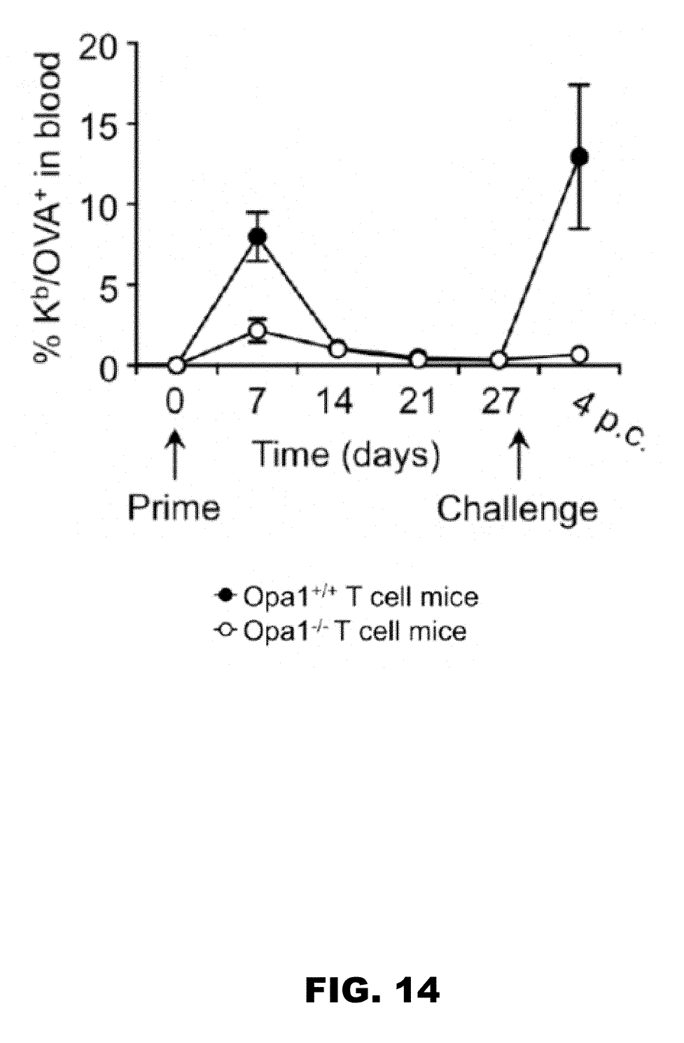

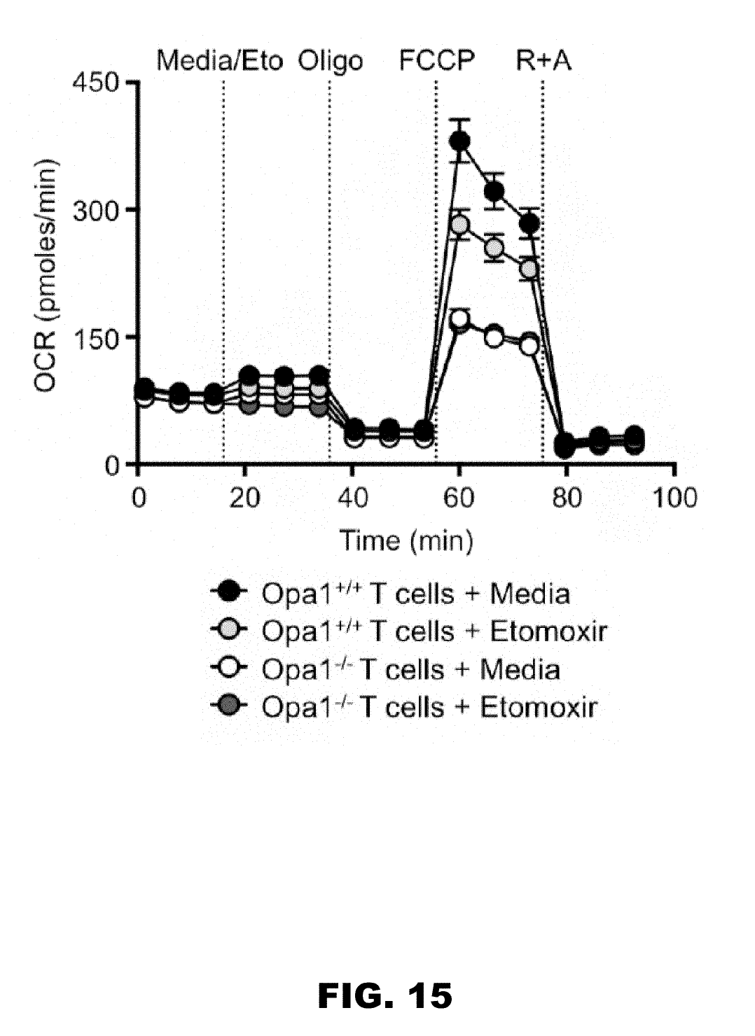

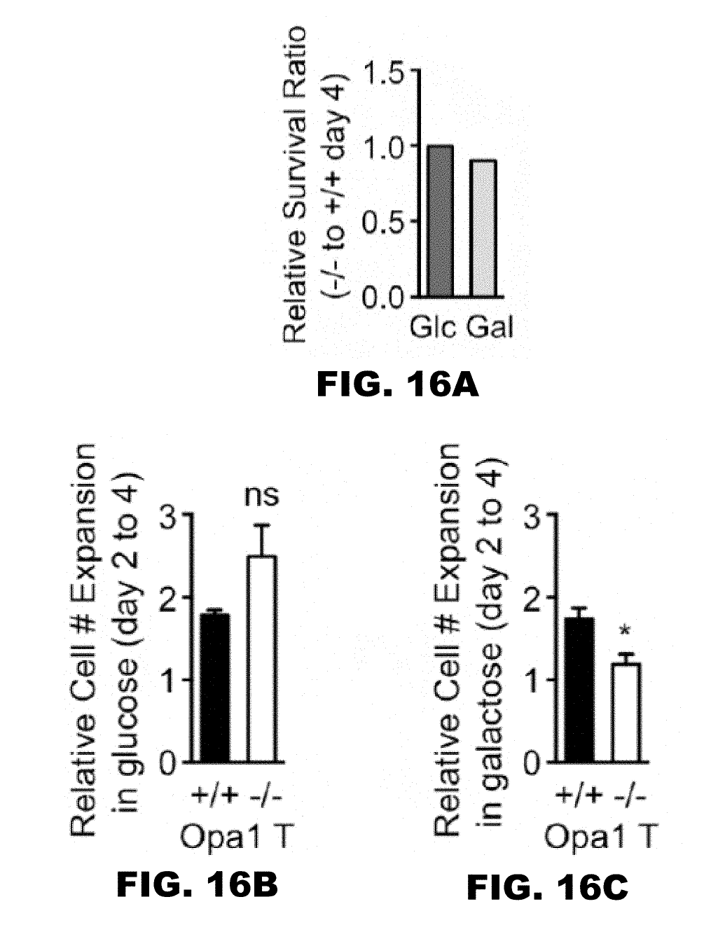

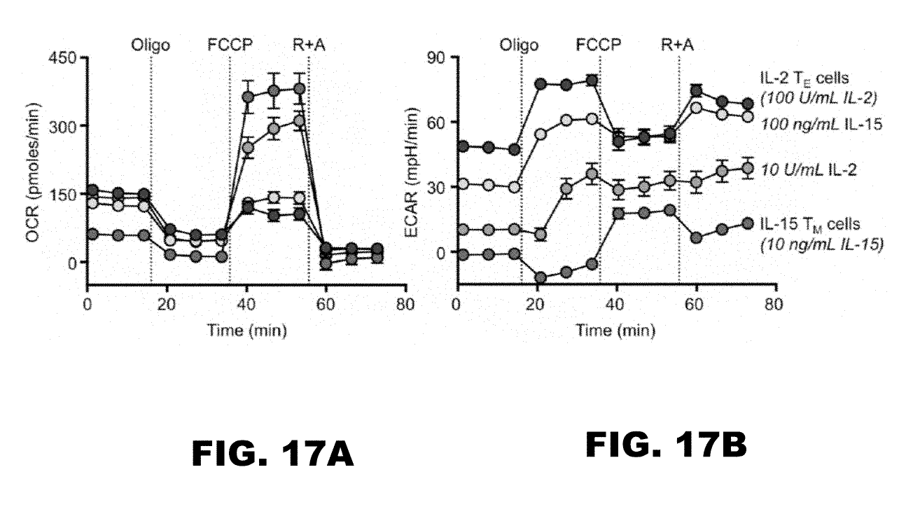

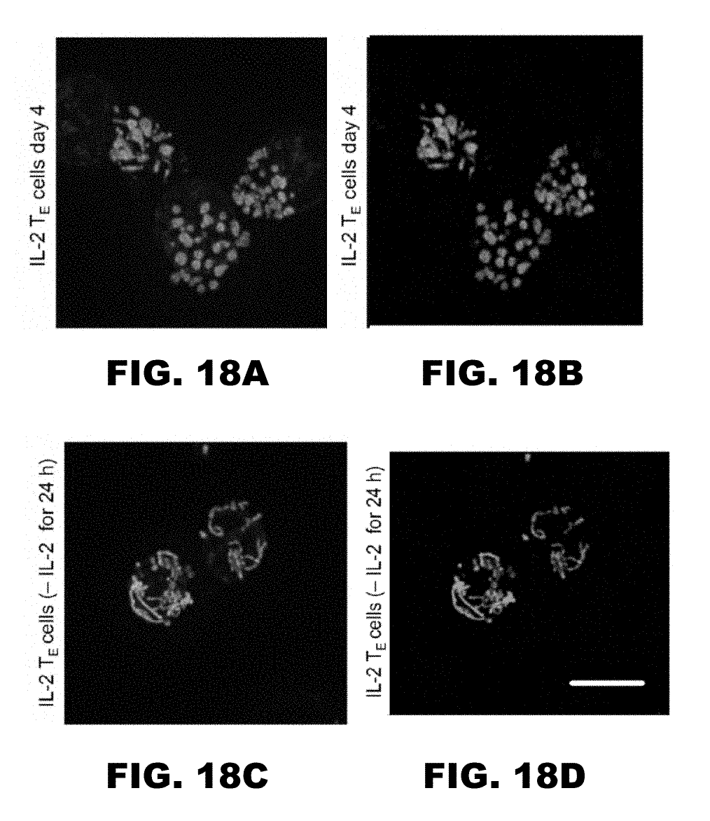

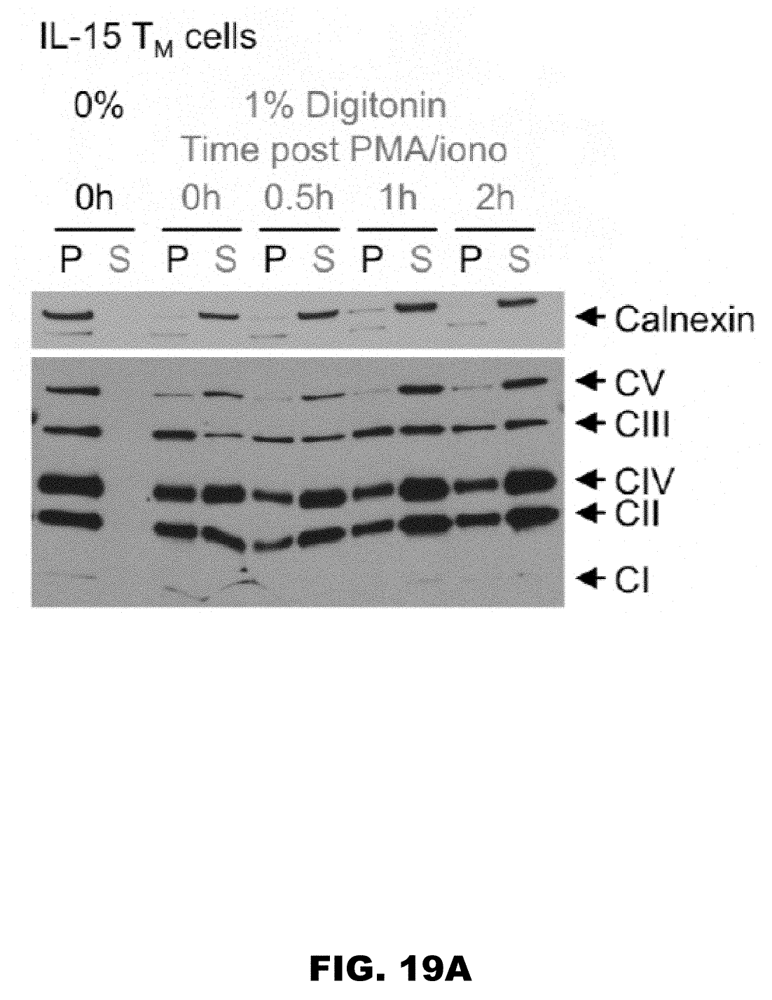

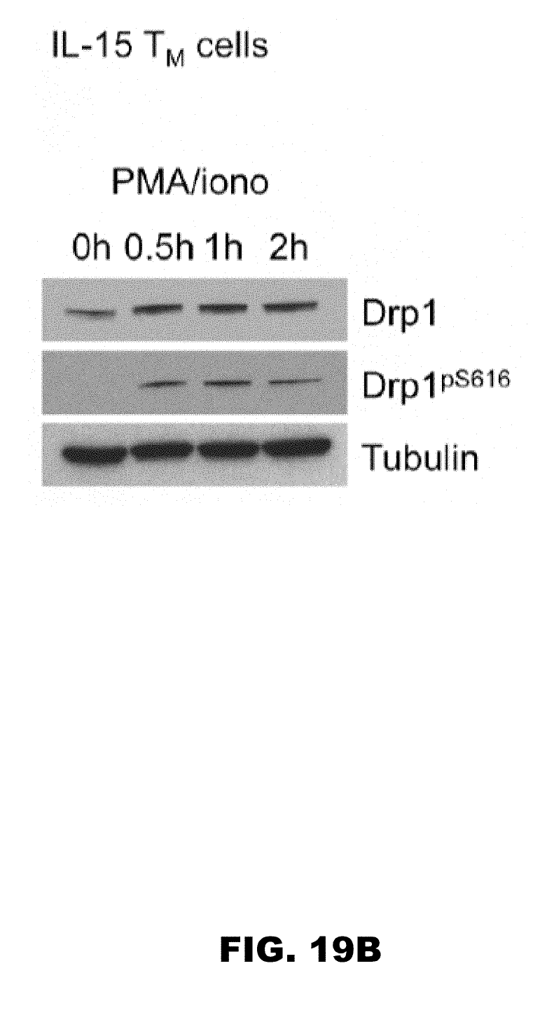

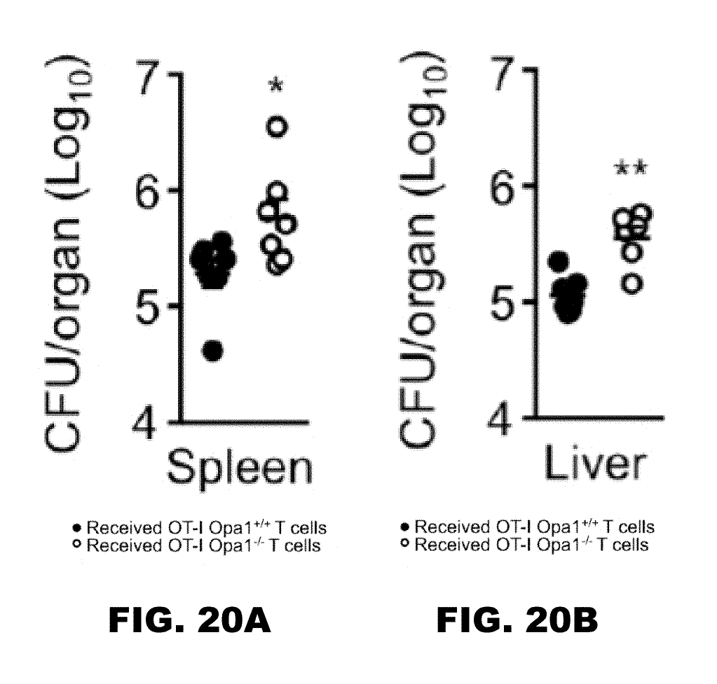

Although both Opa1^{+/+} and Opa1^{-/-} IL-2 T_{E} cells could equally engage FAO (FIG. 5D), we observed that ECAR was significantly augmented in the Opa1^{-/-} cells both in vitro and ex vivo (FIG. 6A). Furthermore, unlike control cells, we observed no additional drop of OCR in Opa1^{-/-} IL-2 T_{E} cells after the addition of oligomycin (FIG. 6B), suggesting that in the absence of Opa1, only FAO supports OXPHOS, and that oxidation of other substrates, such as glucose-derived pyruvate, are not utilized for mitochondrial ATP production in this setting. We cultured Opa1^{+/+} and Opa1^{-/-} IL-2 T_{E} cells with ¹³C-labeled glucose and traced ¹³C into TCA cycle metabolites. We found that while the percent of ¹³C-labeled pyruvate was higher in the Opa1^{-/-} T cells, the frequency of ¹³C-labeled TCA cycle intermediates was significantly reduced in the Opa1^{-/-} T cells compared to controls (FIG. 6C, FIG. 12A), a result that is supported by their higher ECAR (FIG. 6A). These data suggested that without mitochondrial fusion, pyruvate is preferentially secreted as lactate, rather than oxidized in the mitochondria. Therefore, we questioned whether FAO was a ‘default’ pathway for mitochondria in a resting, or fused state (i.e. Opa1 sufficiency), and that the induction of aerobic glycolysis is a major downstream effect of fission (i.e. Opa1 deficiency). If this were the case, then a balance between fission and fusion, modulated by proteins such as Opa1, could act as a primary signal to dictate the metabolic phenotype of T cells. In support of this idea, T cells from polyclonal T cell-conditional deleted Opa1 animals had higher ECAR and an increased proportion of CD8 T cells with an activated effector phenotype in the basal state based on surface marker expression (FIG. 12B).

Opa1 is critical for inner mitochondrial membrane fusion, but also for other processes like cristae remodeling (Frezza et al., 2006, Cogliati et al., 2013, Varanita et al., 2015). We observed major changes in cristae morphology in the Opa1^{-/-} T cells (FIG. 2C). Given the importance of Opa1 function in T_{M} cell development (FIG. 2), we further assessed cristae morphology in T_{E} and T_{M} cells isolated ex vivo after LmOVA infection (FIG. 6D), as well as in IL-2 T_{E} and IL-15 T_{M} cells (FIG. 6E), and found that T_{E} cells had many cristae with what appeared to be slightly wider, or more loosely organized intermembrane space, than T_{M} cells. It has been found that Opa1 overexpression induces cristae tightening and close association of ETC complexes in the inner mitochondrial membrane (Cogliati et al., 2013, Civiletto et al., 2015). Therefore, we surmised that in the absence of Opa1, cristae disorganization leads to dissociation of ETC complexes, and subsequently less efficient ETC activity, in T cells (FIG. 2D, FIG. 2E). We assessed OCR after oligomycin in relation to OCR after rotenone plus antimycin A treatment (i.e. proton leak), which indicates the coupling efficiency of OXPHOS with mitochondrial ATP production. Consistent with decreased OXPHOS efficiency, we observed increased proton leak in Opa1^{-/-} T cells compared to control cells (FIG. 6F). This was also true for ex vivo isolated T_{E} cells when compared to T_{M} cells (FIG. 6G), as well as IL-2 T_{E} and IL-15 T_{M} cells (FIG. 6H). Together these data suggest that there are cristae differences between T_{E} and T_{M} cells which may contribute to their distinct metabolic phenotypes.

We reasoned that fusion renders tightly configured cristae, which results in closely associated ETC complexes and efficient OXPHOS (Patten et al., 2014), producing conditions that favor the entrance of pyruvate into the TCA cycle. In this situation, NADH generated from the TCA cycle is able to easily donate electrons to complex I, which are passed efficiently along the ETC. Our data suggested that this predominantly occurs in T_{M} cells. However, if electron transport across the ETC becomes less efficient, which could be caused by physical separation of the individual complexes due to cristae remodeling via mitochondrial fission, then electrons could linger in the complexes and imbalance redox reactions. NADH levels would build, slowing forward momentum of the TCA cycle. To restore redox balance, cells could augment glycolysis and shunt pyruvate as excreted lactate (i.e. aerobic glycolysis), which would regenerate NAD⁺ from NADH in the cytosol. We speculated that this is what occurs in T_{E} cells. Correlating with this idea, T_{E} and T_{M} cells have different ratios of NAD⁺/NADH (i.e. redox balance), with T_{M} cells maintaining higher NAD⁺/NADH than T_{E} cells. We also showed that NADH levels dramatically rise in T_{M} cells compared to T_{E} cells when exposed to rotenone/antimycin A, indicating that T_{M} cells continually consume more NADH for the purpose of donating electrons to the ETC (van der Windt et al., 2012). Together our data suggested that fission and fusion events regulate cristae remodeling, which could alter ETC efficiency and redox balance, ultimately controlling metabolic adaptations in T cells.

To more thoroughly examine this idea, we assessed cristae morphology in T_{E} and T_{M} cells by EM following TCR stimulation. We hypothesized that if cristae remodeling acts to induce aerobic glycolysis, changes in cristae structure could be visualized following T cell activation. T_{M} cells rapidly augment aerobic glycolysis when restimulated (van der Windt et al., 2013). We activated IL-15 T_{M} cells with αCD3/CD28-conjugated beads (FIG. 6I), or with PMA and ionomycin (FIG. 12F), in the presence or absence of Mdivi-1, to modulate activity of the mitochondrial fission protein Drp1 (Cassidy-Stone et al., 2008). We observed dramatic changes to cristae morphology by EM, with the intermembrane space widening over time in control cells in comparison to drug treated cells. These data are consistent with the hypothesis that fission-induced mitochondrial cristae remodeling supports metabolic reprogramming in T cells.

### Example 7. T_{M} Cells Maintain Tight Cristae with Closely Associated ETC Complexes

Our data suggested that unlike T_{E} cells, T_{M} cells have tight cristae with close association of the ETC complexes. To investigate this biochemically, we treated native lysates of IL-2 T_{E} and IL-15 T_{M} cells with increasing concentrations of digitonin to disrupt all cellular membranes (including mitochondrial). The crude membrane-bound fraction was separated from solubilized proteins by centrifugation. Both pellet and soluble supernatant fractions were loaded on a denaturing reducing gel and then probed for various mitochondrial proteins by western blot. We found that mitochondria in IL-2 T_{E} cells were susceptible to digitonin disruption, indicated by the fact that ETC complex proteins became less detectable in the pellet, and amplified in the soluble fraction, in 0.5% detergent (FIG. 6J, FIG. 12G, FIG. 12H). This was in contrast to IL-15 T_{M} cells, where ETC proteins did not solubilize to the same extent as those in IL-2 T_{E} cells into the supernatant, even when 2% digitonin was used. To investigate whether this phenomenon was unique to the mitochondrial compartment, we also probed for the ER integral protein calnexin and found that it solubilized similarly in 0.5% digitonin in both cell types. Overall these data suggest that there is more exposed mitochondrial membrane between proteins in IL-2 T_{E} cells than in IL-15 T_{M} cells, and correlate with the idea that T_{M} cells have tight cristae which would yield efficient ETC activity, while T_{E} cells have looser cristae with less efficient ETC activity, ultimately supporting their distinct metabolic phenotypes.

### Example 8. Mitochondrial Fission in Activated Immune Cells Facilitates Aerobic Glycolysis

Our data suggested that cristae remodeling, through fission and fusion events, was a mechanism to regulate efficient OXPHOS and FAO in T_{M} cells, as well as the induction of aerobic glycolysis in T_{E} cells. To more directly test this idea, we assessed ECAR of IL-15 T_{M} cells that were stimulated with αCD3/28-conjugated beads in the presence or absence of Mdivi-1. We found that when mitochondrial fission protein Drp1 was inhibited with Mdivi-1, T cell activation did not robustly increase aerobic glycolysis when compared control cells (FIG. 6K), which correlated with our EM data (FIG. 6I). Since fission can be associated with cell division, we wanted to test our idea in a non-proliferating cell type that substantially augments aerobic glycolysis upon stimulation (Krawczyk et al., 2010, Everts et al., 2014). We stimulated bone marrow derived dendritic cells (BM-DCs) and macrophages (BM-Macs) with lipolysaccharide (LPS) with or without interferon (IFN)-γ in the presence or absence of Mdivi-1 and measured ECAR. Aerobic glycolysis was curtailed in both BM-DCs and BM-Macs following stimulation when Drp1 was inhibited (FIG. 6L, FIG. 6M). The blunted ECAR in the Mdivi-1 treated cells correlated with significantly decreased nitric oxide synthase 2 (Nos2) protein expression in the BM-Macs (FIG. 6N), indicating that their activation was also repressed. These data indicate that cristae remodeling and/or fission acts as a signal to drive the induction of aerobic glycolysis, and subsequent cellular activation via Drp1.

### Example 9. Mitochondrial Dynamics/Fission in the Control of TH17/Treg Balance

CD4+ T cells differentiate into a variety of effector and regulatory T cell subsets, which show extremely diverse functions and metabolic configurations; where the inflammatory Th1, Th2, and Th17 T cell subsets utilize glycolysis while regulatory T cells (Treg) show a requirement for lipid metabolism, glycolysis, and oxidative phosphorylation. The engagement of specific metabolic pathways not only supports T cell differentiation, but specific effector functions cannot proceed without adopting the correct metabolism. Hence, reprogramming metabolic pathways in T cells appears as an exciting therapeutic strategy against immune diseases.

It was previously demonstrated that increasing mitochondrial fission in T cells by specific deletion of the profusion protein Opa1 reduces electron transport chain (ETC) efficiency and pyruvate oxidation into mitochondria, increasing aerobic glycolysis and the generation of effector T cells. In contrast, increasing mitochondrial fusion by Opa1 overexpression or treating cells with the profusion drugs Mdivi-1 and M1 facilitates ETC activity and pyruvate entrance into mitochondria, triggering the generation of long-lived memory T cells. Thus, genetic or pharmacological modulation of mitochondrial morphology and function impacts cellular metabolism and the fate of effector and memory T cells.

Here this concept is extended to investigate the role of mitochondrial dynamics in the control of T cell differentiation. Our results show that mitochondrial dynamics controls the differentiation of the distinct regulatory and effector T cell subsets. Genetic or pharmacological inhibition of mitochondrial fission reduced IL17 secretion while concomitantly increasing Foxp3 expression, a marker of regulatory T cells, thus altering the balance between regulatory and Th17 T cells in favor of regulatory T cells. The identification of mitochondrial fission and its main player Drp1 as a therapeutic target to control Th17 and regulatory T cell balance opens novel avenues for treating immune diseases associated with increased pro-inflammatory conditions such as rheumatoid arthritis, multiple sclerosis, autoimmunity disorders or psoriasis.

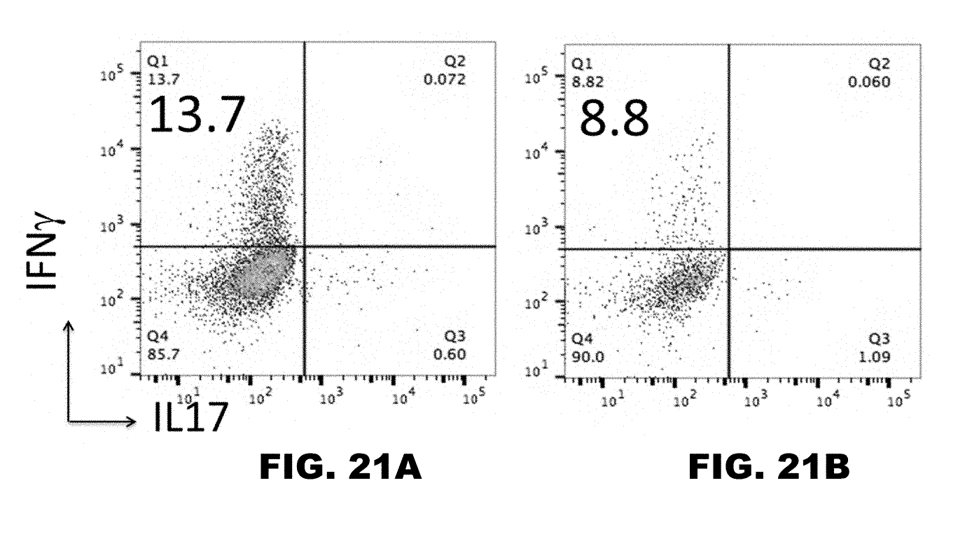

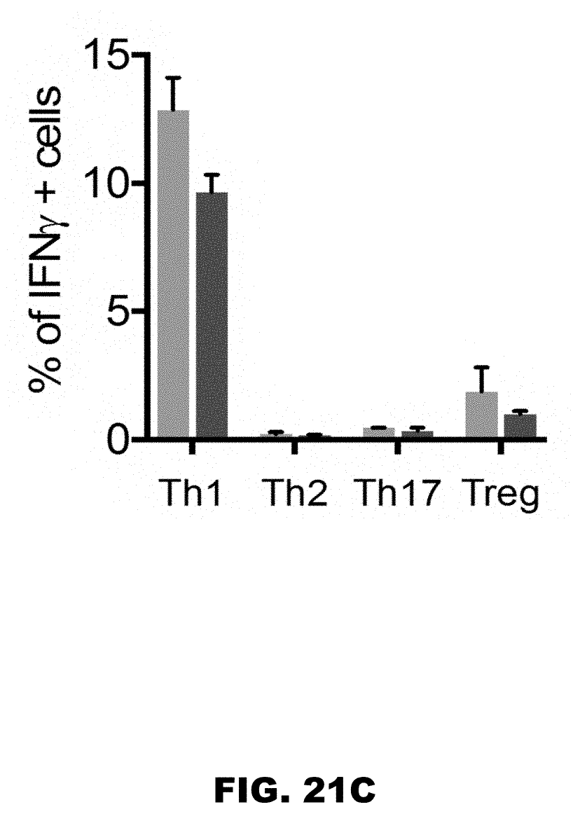

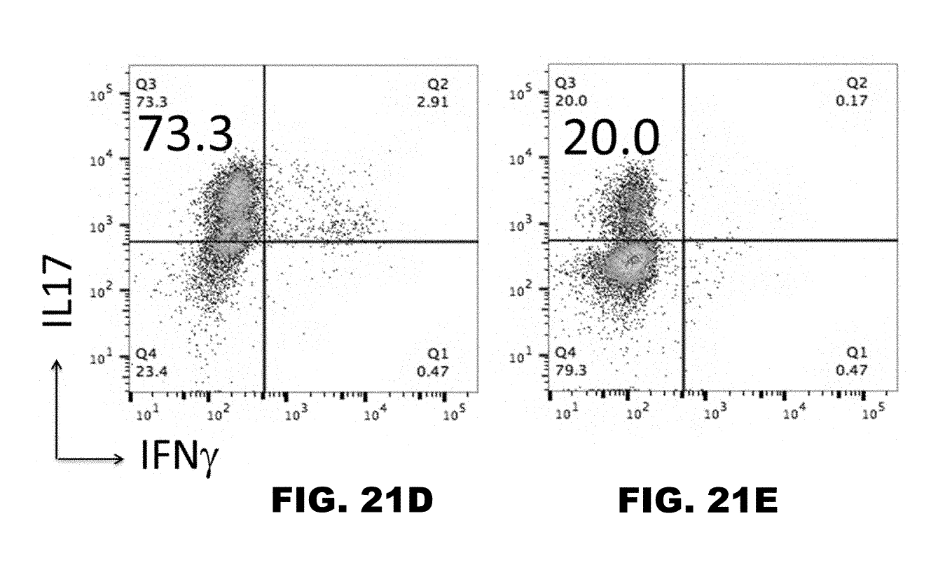

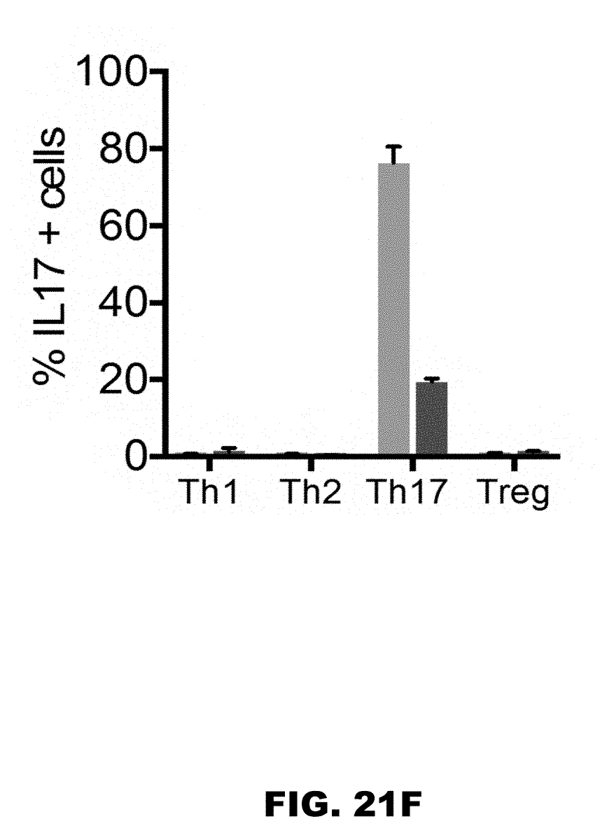

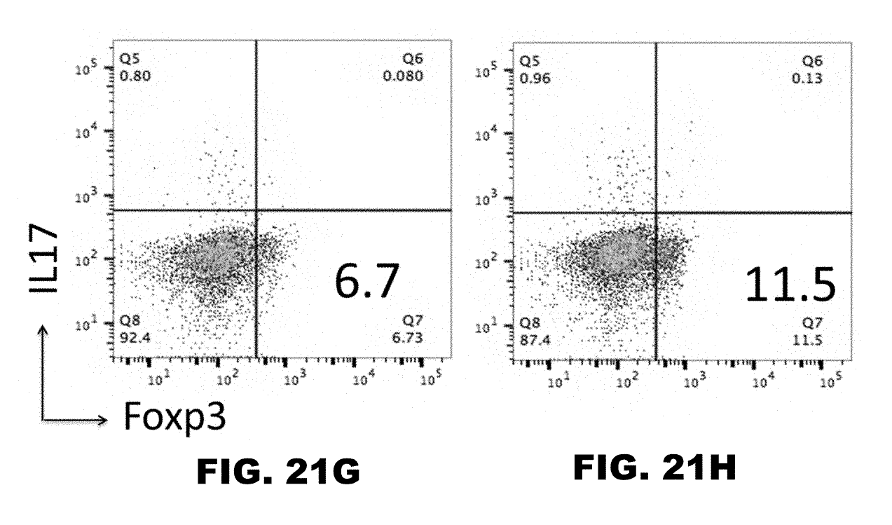

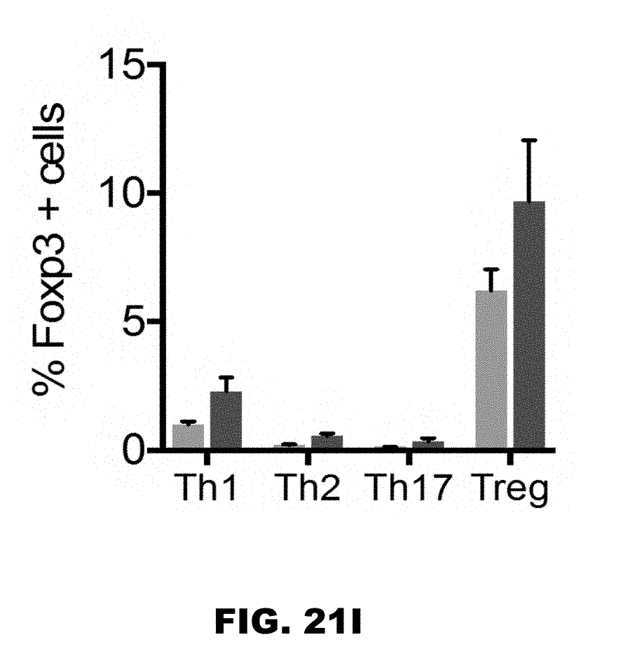

To investigate whether mitochondrial dynamics regulates T cell differentiation we isolated naive CD4+ T cells and skewed them in vitro into Th1, Th2, Th17 and Treg cells by using a combination of cytokines and blocking antibodies in the presence or absence of the profusion drugs Mdivi-1 and M1. Pharmacological inhibition of mitochondrial fission reduced IFN-γ production in Th1 culture conditions (FIG. 21A, FIG. 21B, FIG. 21C). The profusion treatment significantly reduced IL-17 cytokine expression in Th17 polarization conditions (FIG. 21D, FIG. 21D, FIG. 21F), whereas it increased the level of the regulatory T cell lineage transcription factor Foxp3 in the polarizing conditions tested (FIG. 21G, FIG. 21H, FIG. 21F). Hence, boosting organelle fusion through the combined usage of the profusion drug M1 and the mitochondrial fission specific inhibitor Mdivi-1 reduces the pro-inflammatory cytokine expression of T cells while increase their regulatory fate.

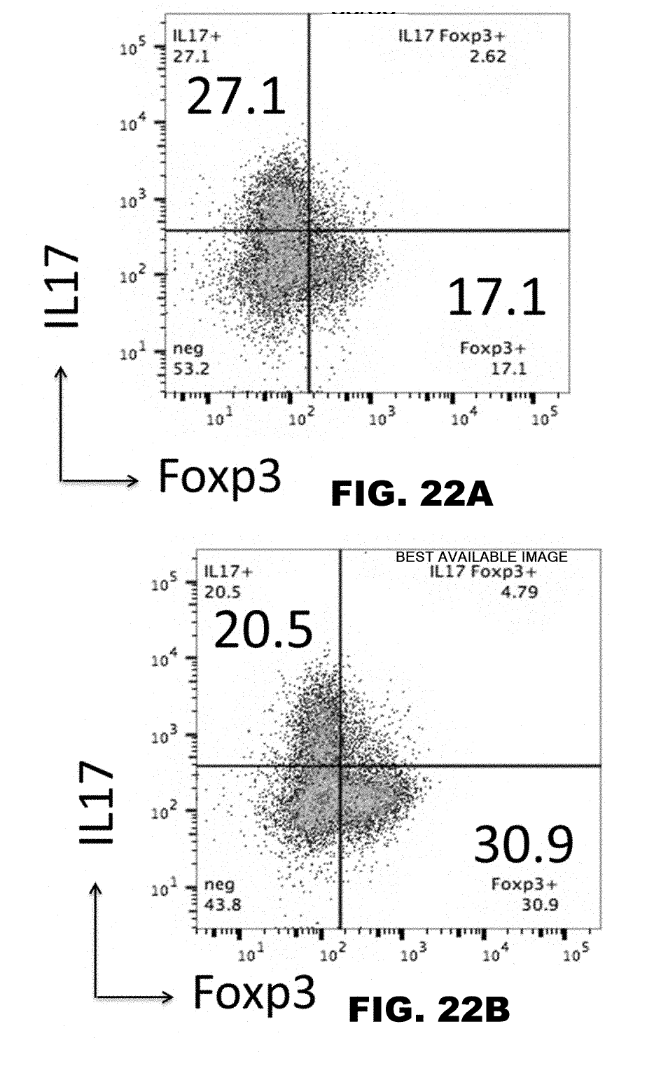

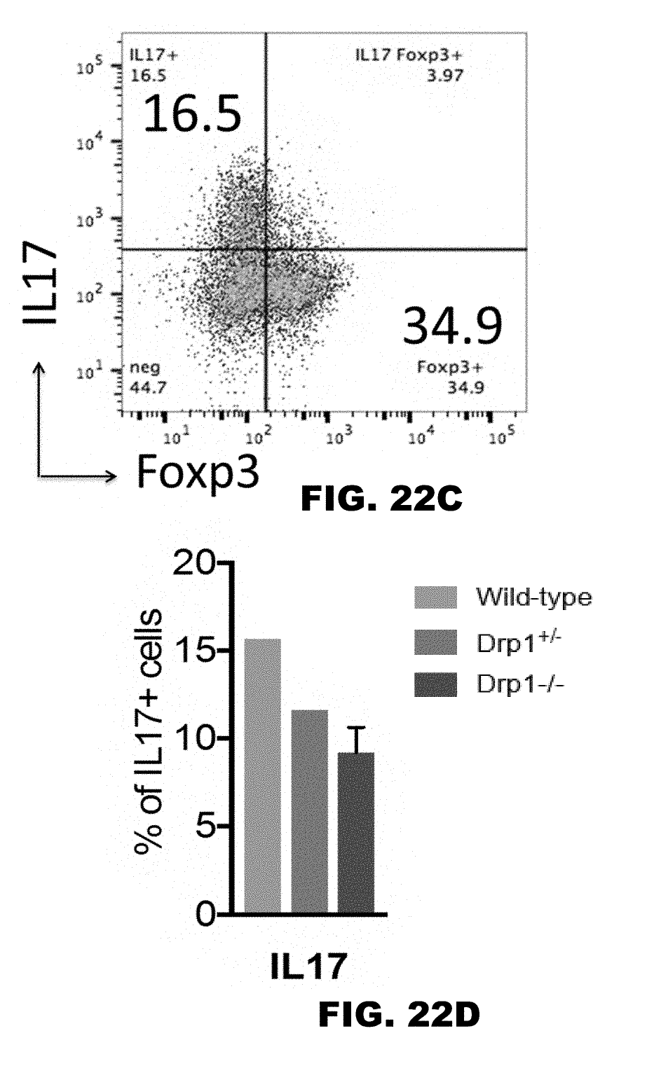

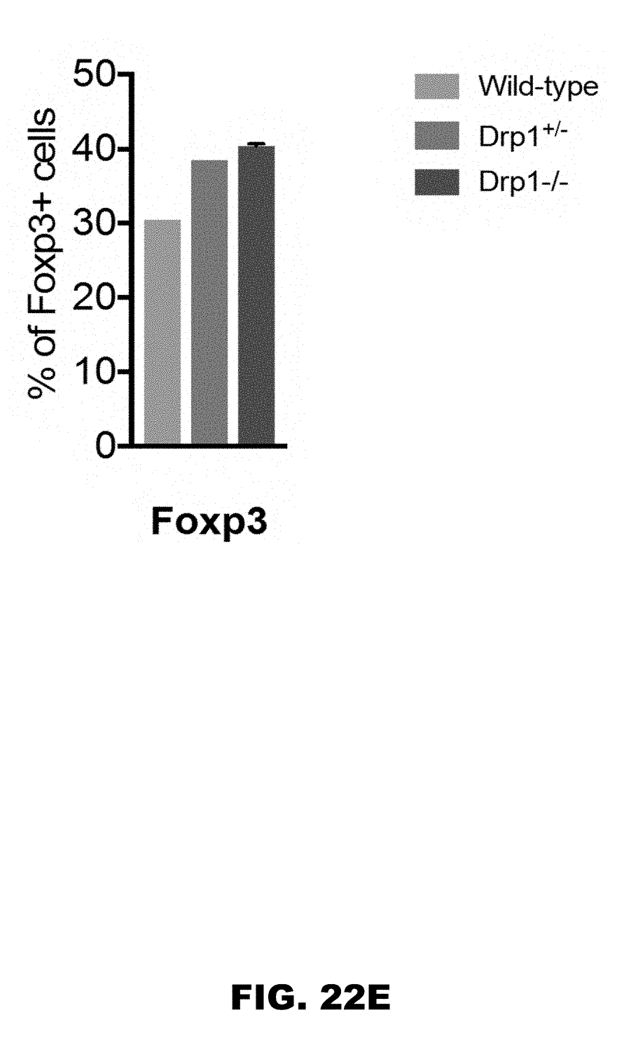

To get more specific insights into the role of mitochondrial fission in regulating the balance between pro-inflammatory and effector T cell subsets, a genetic mouse model to specifically deplete the mitochondrial fission protein Drp1 in T cells (CD4CreDrp1fl/fl, Drp1−/−) was used. Targeting Drp1 reduces IL17 production while increasing Foxp3 expression in naive Drp1−/− CD4+ T cells skewed into pro-inflammatory Th17 T cells (FIG. 22A-E), supporting the role of mitochondrial fission and Drp1 in controlling the balance between regulatory and effector fate of T cells.

### Discussion for the Examples

Although T_{M} cells rely on FAO for development and survival, precisely why T_{M} cells utilize FAO and the signals that drive the induction of aerobic glycolysis in T_{E} cells remain unclear. Our data suggest that manipulating the structure of a single organelle can have profound consequences that impact metabolic pathway engagement and ultimately, the differentiation of a cell. We found that Opa1 regulated tight cristae organization in T_{M} cells, which facilitated efficient ETC activity and favorable redox balance that allowed continued entrance of pyruvate into mitochondria. We originally hypothesized that Opa1 would be an obligate requirement for FAO. However, we found that Opa-1^{-/-} IL-2 T_{E} cells and ex vivo T_{E} cells generated during infection utilized FAO to the same level as cells expressing Opa1. While this was true for T_{E} cells, this may not be the case for T_{M} cells, whose survival is severely impaired in vitro and in vivo when deficient in Opa1. It is possible that Opa1^{-/-} T cells are unable to form T_{M} cells because they cannot efficiently engage FAO under the metabolic constraints imposed during T_{M} cell development. Previous studies point to the existence of a ‘futile’ cycle of fatty acid synthesis (FAS) and FAO within T_{M} cells (O'Sullivan et al., 2014, Cui et al., 2015) whereby carbon derived from glucose oxidation is used to build fat that is subsequently burned by mitochondria for fuel. T_{M} cells have a lower overall metabolic rate than T_{E} cells, and tightly configured cristae might be important to ensure that any pyruvate generated will efficiently feed into the TCA cycle not only for reducing equivalents, but also for deriving citrate for FAS. Without tight cristae and efficient ETC activity, electrons may loiter in the complexes, causing more ROS, which could be damaging (Chouchani et al., 2014), but also provide signals that drive cell activation (Sena et al., 2013).

We did not observe a defect in T_{M} cell survival in Mfn1 and Mfn2 deficient T cells, but this does not exclude the possibility that OMM fusion or additional activities ascribed to each of these proteins are not important. Mfn1 and Mfn2 form both homotypic and heterotypic interactions, suggesting that in the absence of one protein, the other can compensate (Chen et al., 2003). Our preliminary data assessing Mfn1/2 expression in Mfn1^{-/-} and Mfn2^{-/-} T cells indicate that this might be occurring (data not shown). However, our results clearly show that unlike Opa1^{-/-} T cells, in vitro cultured Mfn1^{-/-} or Mfn2^{-/-} T cells do not have a survival defect when differentiated in IL-15 (FIG. 2A, FIG. 2B), even though, like Opa1^{-/-} T cells, they are more glycolytic and OXPHOS-impaired compared to controls (data not shown). Further investigation using Mfn1/2 double knockouts is underway in our laboratory to examine whether a lack of both proteins, and presumably total OMM fusion, impairs T_{M} cell development in similar fashion as deficiency in inner membrane fusion. Our imaging data showed that T_{M} cells maintained extended fused mitochondrial networks, suggesting that OMM fusion also has a compulsory role in T_{M} cell development. However, unlike Opa1, retroviral expression of Mfn1 and Mfn2 did not confer a T_{M} cell phenotype in T_{E} cells. This could be due to the fact that an increase in OMM fusion, without a concomitant increase in inner membrane fusion, would still yield an overall loose cristae morphology and redox state that by default, results in sustained excretion of lactate.

The question of what signals drive T cell structural remodeling of mitochondria in the first place still remains. In the case of T_{M} cell development, initial withdrawal of activating signals and growth factors may induce fusion, consistent with previous reports that starvation induces mitochondrial hyperfusion (Rambold et al., 2011b, Rambold et al., 2015), an effect we also observe in T_{E} cells after IL-2 withdrawal (data not shown). However, pro-survival signals from cytokines such as IL-15 or IL-7 are needed to sustain T_{M} cell viability and metabolically remodel these cells for FAS and FAO via increased CPT1a (van der Windt et al., 2012) and aquaporin 9 expression (Cui et al., 2015). Factors such as these may enforce the fused state and would be consistent with our observations that activated T cells subsequently cultured in IL-15 become more fused over time (FIG. 1D). Another possibility is that Opa1 is activated via sirtuin 3 (SIRT3) under metabolically stressful conditions (Samant et al., 2014). Sirtuins are post-translational modifiers that are activated by NAD⁺, directly tying their activity to the metabolic state of the cell (Houtkooper et al., 2012, Wang and Green, 2012). Our previous work demonstrated that the available NAD⁺ pool is higher in T_{M} cells (van der Windt et al., 2012), which could correlate with this scenario.

In T_{E} cells we see an immediate activation of Drp1, prior to seeing a fissed phenotype, and inhibition of Drp1 prevents ECAR induction after activation. TCR signals induce Ca²⁺ flux that activates the phosphatase activity of calcineurin (Smith-Garvin et al., 2009), which in turn dephosphorylates Drp1 at Ser637, leading to its activation (Cereghetti et al., 2008). Initial Drp1 activation could facilitate some level of fission and cristae remodeling, tipping off aerobic glycolysis via the initial shunting of pyruvate to lactate. Our data (FIG. 1E) showed that Drp1 is phosphorylated at its activating site Ser616 at day 1 after activation, which preceded recognizable mitochondrial fragmentation (FIG. 1C). Our preliminary data did not show overt mitochondrial fragmentation in the initial hours after TLR stimulation of DC or macrophages (data not shown), but this does not exclude the possibility that Drp1 is actively mediating more subtle changes to mitochondrial structure that are not discernable by confocal microscopy. For example, Drp1 also has been found to affect cristae structure by altering the fluidity of the mitochondrial membrane (Benard et al., 2007, Benard and Rossignol, 2008). Although Drp1 has been implicated in mitochondrial positioning at the immune synapse (Baixauli et al., 2011), lymphocyte chemotaxis (Campello et al., 2006), and ROS production (Roth et al., 2014) during T cell activation, our data suggest that in addition to these processes, fission underlies the reprogramming of cells to aerobic glycolysis.

We show that IL-2 T_{E} cells have a mitochondrial structure that is more susceptible to digitonin disruption when compared to IL-15 T_{M} cells, which suggests a more exposed membrane with less densely packed protein complexes. This relatively enhanced permeability however, does not mean that their mitochondria are damaged, or unable to function. In fact, although T_{E} cells have less efficient OXPHOS in terms of how it is coupled to ATP synthesis, T_{E} cells are very metabolically active with high OCR and ECAR (Chang et al., 2013, Sena et al., 2013). Our experiments involving pharmacological enforcement of mitochondrial fusion promoted OCR and SRC (and ECAR, albeit to a lesser extent) in IL-2 T_{E} cells. The drug modified cells maintained full T_{E} cell function with no effect on their cytolytic ability or proliferation, but possessed enhanced cytokine expression. Fusion and/or cristae tightening boosted the T_{E} cells' oxidative capacity, endowing them with longevity and persistence, while their higher aerobic glycolysis supported increased cytokine production, which may explain their superior antitumor function.

Our data suggest a model where morphological changes in mitochondria are a primary signal that shapes metabolic reprogramming during cellular quiescence or activation. We speculate that fission associated expansion of cristae as a result of TCR stimulation physically separates ETC complexes, decreasing ETC efficiency. With delayed movement of electrons from complex I down the ETC, NADH levels rise in the mitochondria, slowing forward momentum of the TCA cycle and cause an initial drop in ATP. To correct redox balance, cells will export pyruvate to lactate to regenerate NAD⁺ in the cytosol, which can enter the mitochondria through various shuttles to restore redox balance (Dawson, 1979) and increase flux through glycolysis to restore ATP levels, all contributing to the Warburg effect in activated T cells. When cristae are tightly configured, the ETC works efficiently and maintains entrance of pyruvate into the mitochondria with a favorable redox balance. In this case, cristae morphology as a result of fusion directs T_{M} cell formation and retains these cells in a quiescent state. Thus, mitochondrial dynamics control the balance between metabolic pathway engagement and T cell fate.

### Methods for the Examples

Mice and Immunizations:

C57BL/6, C57BL/6 CD45.1, C57BL/6 CD90.1, photo-activatable mitochondria (PhAM), and major histocompatibility complex (MHC) class I-restricted OVA specific TCR OT-I transgenic mice were purchased from The Jackson Laboratory. Mfn1 and Mfn2 conditional floxed mice were obtained from Dr. David C. Chan (California Institute of Technology, Pasadena, Calif.). Opa1 conditional floxed mice were obtained from Dr. Hiromi Sesaki (Johns Hopkins University School of Medicine, Baltimore, Md.). All conditional floxed mice were crossed to OT-I CD4 Cre transgenic mice to generate OT-I Mfn1^{F/F} CD4 Cre, OT-I Mfn2^{F/F} CD4 Cre, and OT-I Opa1^{F/F} CD4 Cre mice. All mice were bred and maintained under specific pathogen free conditions under protocols approved by the AAALAC accredited Animal Studies Committee of Washington University School of Medicine, St. Louis, Mo. USA and the Animal Welfare Committee of the Max Planck Institute of Immunobiology and Epigenetics Freiburg, Germany. Age matched mice were injected intraperitoneally (i.p.) or intravenously (i.v.) as indicated with a sublethal dose of 1×10⁶ colony forming units (CFU) of recombinant Listeria monocytogenes expressing OVA deleted for actA (LmOVA) for primary immunizations and challenged with 5×10⁷ CFU for secondary immunizations. For tumor experiments, 1×10⁶ EL4 lymphoma cells expressing OVA (EL4-OVA) were injected subcutaneously (s.c.) into the right flank of mice.

Cell Culture and Drug Treatments:

OT-I splenocytes were activated with OVA-peptide (SIINFEKL (SEQ ID NO:1), New England Peptide) and IL-2 (100 U/mL) for 3 days and subsequently cultured in the presence of either IL-2 or IL-15 (10 ng/mL) for an additional 3 days in TCM (RPMI 1640 media supplemented with 10% FCS, 2 mM L-glutamine, 100 U/mL penicillin/streptomycin, and 55 μM β-mercaptoethanol). For drug treatment experiments, vehicle control (DMSO) or 10 μM Mdivi-1+20 μM M1 (Sigma) were added to cultures daily starting on day 3. For in vitro survival assays, cells were activated for 3 days as described, then cultured in either IL-2 at 5×10⁴ cells/mL or IL-15 at 1×10⁵ cells/mL in 96 well round bottom plates. Survival was analyzed by 7AAD exclusion using flow cytometry. Bone marrow cells were differentiated for 7 days into BM-Macs by culturing in complete medium (RPMI 1640 supplemented with 10% FBS, 100 U/mL penicillin/streptomycin, 2 mM L-glutamine) with 20 ng/mL mouse macrophage colony-stimulating factor (M-CSF; PeproTech) or into BM-DCs using 20 ng/mL granulocyte-macrophage colony-stimulating factor (GM-CSF; PeproTech). BM-Macs and BM-DCs were stimulated using 20 ng/mL LPS (Sigma), 50 ng/mL IFN-γ (R&D Systems), or 20 ng/mL IL-4 (PeproTech). BM-DCs were cultured in 5 ng/mL GM-CSF during stimulation experiments.

Flow Cytometry and Spinning Disk Confocal Microscopy:

Fluorochrome-conjugated monoclonal antibodies were purchased from eBioscience, BD Pharmingen, or Biolegend and staining performed as previously described (Chang et al., 2015). OVA-specific CD8⁺ T cells from spleen, lymph node, or blood were quantified by direct staining with H2-K^{b}OVA257-264 (K^{b}OVA) MHC-peptide tetramers. MitoTracker, TMRE, CMxROS, MitoSOX, and Hoechst staining was performed according to the manufacturer's instructions (Life Technologies). Nos2 protein levels in BM-Macs were quantified after fixation and permeabilization using the transcription factor staining buffer set (eBioscience) and a directly conjugated antibody against Nos2 (clone CXNFT, eBioscience). Cells were collected on FACS Calibur, Canto II, LSR II, and Fortressa flow cytometers (BD Biosciences) and analyzed using FlowJo (TreeStar) software. Cells were sorted using a FACS Aria II. Cells were imaged live on glass bottom dishes coated with fibronectin or poly-D-lysine (Sigma) in TCM containing IL-2 or IL-15 (MatTek) using a LSM 510 META confocal scanning microscope (Zeiss), an Olympus Confocal Microscope FV1000, or a Zeiss spinning disk confocal with a Evolve (EMCCD) camera. Cells were kept in a humidified incubation chamber at 37° C. with 5% CO₂ during image collection. Images were deconvolved and analyzed using ImageJ (NIH). Brightness and contrast were adjusted in Adobe Photoshop CS.

Transmission Electron Microscopy:

Cells were fixed in 2% paraformaldehyde, 2.5% glutaraldehyde in 100 mM sodium cocodylate containing 0.05% malachite green. Following fixation, samples were washed in cocodylate buffer and post fixed in 1% osmium tetroxide. After extensive washing in H₂O, samples were stained with 1% aqueous uranyl acetate for 1 hour and washed again. Samples were dehydrated in ethanol and embedded in Eponate 12 resin (Ted Pella). Cut sections were stained with uranyl acetate and lead citrate and then imaged using a JOEL 1200 EX transmission electron microscope equipped with an 8 MP ATMP digital camera (Advanced Microscopy Techniques).

Metabolism Assays:

Oxygen consumption rates (OCR) and extracellular acidification rates (ECAR) were measured in XF media (non-buffered RPMI 1640 containing 25 mM glucose, 2 mM L-glutamine, and 1 mM sodium pyruvate) under basal conditions and in response to 200 μM etomoxir (Tocris), 1 μM oligomycin, 1.5 μM fluoro-carbonyl cyanide phenylhydrazone (FCCP) and 100 nM rotenone+1 μM antimycin A, or 50 ng/mL phorbol 12-myristate 13-acetate (PMA)+500 ng/mL ionomycin (all Sigma) using a 96 well XF or XFe Extracellular Flux Analyzer (EFA) (Seahorse Bioscience). For mitochondrial fission inhibition experiments, cells were plated in XF media containing 10 μM Mdivi-1 or vehicle control (DMSO), followed by injection into port A with XF media, αCD3/CD28-conjugated beads (1 bead/cell; Dynabeads), or 20 ng/mL LPS±50 ng/mL IFN-γ.

Glucose Tracing:

Cells were activated with OVA peptide and cultured in glucose free TCM (prepared with dialyzed FBS) supplemented with 11 mM glucose. On day 3 of culture, cells were washed and cultured overnight in TCM replaced with 11 mM D-[1,2¹³C] labeled glucose. For harvest, cells were rinsed with cold 150 mM ammonium acetate (NH4AcO), and metabolites extracted using 1.2 mL of 80% MeOH kept on dry ice. 10 nM norvaline (internal standard) was added. Following mixing and centrifugation, the supernatant was collected, transferred into glass vials and dried via centrifugal evaporation. Metabolites were resuspended in 50 μL 70% ACN and 5 μL of this solution used for mass spectrometer-based analysis performed on a Q Exactive (Thermo Scientific) coupled to an UltiMate 3000RSLC (Thermo Scientific) UHPLC system. Mobile phase A was 5 mM NH₄AcO, pH 9.9, B was ACN, and the separation achieved on a Luna 3u NH₂ 100 A (150×2.0 mm) (Phenomenex) column. The flow was kept at 200 μL/min, and the gradient was from 15% A to 95% A in 18 min, followed by an isocratic step for 9 min and re-equilibration for 7 min. Metabolites we detected and quantified as area under the curve (AUC) based on retention time and accurate mass 3 p.p.m.) using TraceFinder 3.3 (Thermo Scientific) software.

Adoptive Transfers:

For in vivo memory T cell experiments, ≦1×10⁴ OT-I⁺ CD8⁺ cells/mouse from donor splenocytes were transferred intravenously (i.v.) into congenic recipient mice. Blood samples or spleens were collected at indicated time points and analyzed by flow cytometry. For in vivo survival experiments, 1-2×10⁶ day 6 IL-2 T_{E} treated cells/mouse were injected i.v. into naïve C57BL/6 mice. Cells were recovered two days later from the spleen or lymph nodes and analyzed by flow cytometry or isolated from spleens >3 weeks 6 days after LmOVA infection. For adoptive cellular immunotherapy experiments, 1-5×10⁶ day 6 IL-2 T_{E} treated cells/mouse were injected i.v. into previously EL4-OVA tumor inoculated mice and measured for tumor volume growth.

RT-PCR and Western Blotting:

RNA isolations were done by using the RNeasy kit (Qiagen) and single-strand cDNA was synthesized using the High Capacity cDNA Reverse Transcription Kit (Applied Biosystems). Genomic DNA was extracted using the QIAamp DNA micro kit (Qiagen) to determine mtDNA/nDNA ratios. All RT-PCR was performed with Taqman primers using an Applied Biosystems 7000 sequence detection system. The expression levels of mRNA were normalized to the expression of a housekeeping gene (β-actin). For western blot analyses, cells were washed with ice cold PBS and lysed in 1× lysis buffer (Cell Signaling Technologies) supplemented with 1 mM PMSF. Samples were freeze-thawed 3 times and centrifuged at 20,000×g for 10 min at 4° C. Cleared protein lysate was denatured with LDS loading buffer for 10 min at 70° C. For native lysis, cells were resuspended in native lysis buffer (Life Technologies), supplemented with increasing percentages of digitonin, MgCl, and micrococcal nuclease. After nuclease incubation at RT for 1 h, lysates were cleared by centrifugation at 20,000×g for 30 min at 4° C. For mitochondrial membrane solubilization analyses, both the cleared supernatant and pellet were denatured with LDS loading buffer for 10 min at 70° C. Samples were run on precast 4-12% bis-tris protein gels (Life Technologies). Proteins were transferred onto nitrocellulose membranes using the iBLOT 2 system (Life Technologies). Membranes were blocked with 5% w/v milk and 0.1% Tween-20 in TBS and incubated with the appropriate antibodies in 5% w/v BSA in TBS with 0.1% Tween-20 overnight at 4° C. The following antibodies were used: Opa1 (BD), rodent OXPHOS complex proteins cocktail (Abcam), Calnexin (Santa Cruz), and 13-Actin, Mfn2, Drp1, Drp1^{pS616} (Cell Signaling Technologies). All primary antibody incubations were followed by incubation with secondary HRP-conjugated antibody (Pierce) in 5% milk and 0.1% Tween-20 in TBS and visualized using SuperSignal West Pico or femto Chemiluminescent Substrate (Pierce) on Biomax MR film (Kodak).

Retroviral Transduction:

Activated OT-I splenocytes were transduced with control (empty vector) or Mfn1, Mfn2, Opa1 expressing retrovirus by centrifugation for 90 minutes in media containing hexadimethrine bromide (8 μg/mL; Sigma) and IL-2 (100 U/mL). GFP or human CD8 were markers for retroviral expression.

Cytotoxicity Assay:

EL4-OVA tumor cells were pre-treated with 100 U/mL murine IFN-γ for 24 hours before use. To generate target cells, 1×10⁶ tumor cells were labeled with 0.5 μM Cell Proliferation Dye e670 (eBioscience) in PBS for 8 minutes at room temperature, washed twice with PBS and 10,000 cells were seeded per well in 96-well round bottom plates. IL-2 T_{E} cells treated with DMSO or M1+Mdivi-1 were co-cultured with target cells at the indicated effector/target cell ratios and incubated for 12 hours at 37° C. in 5% CO₂. To generate reference cells, 1×10⁶ tumor cells were labeled with 5 μM Cell Proliferation Dye e670 in PBS and incubated on ice. 10,000 reference cells were added before cells were stained with PoPro™-1 dead cell staining dye (Life Technologies). IL-2 T_{E} cell killing efficiency was analyzed by flow cytometry and data defined as percentage of live cells normalized to reference cells.

Statistical Analysis:

Comparisons for two groups were calculated using unpaired two-tailed student's t-tests, comparisons for more than two groups were calculated using one-way ANOVA followed by Bonferroni's multiple comparison tests. Comparisons over time were calculated using two-way ANOVA followed by Bonferroni's multiple comparison tests.

### REFERENCES FOR THE EXAMPLES

Archer, S. L. (2014). Mitochondrial fission and fusion in human diseases. N Engl J Med, 370, 1074.
Baixauli, F., Martin-Cofreces, N. B., Morlino, G., Carrasco, Y. R., Calabia-Linares, C., Veiga, E., Serrador, J. M. & Sanchez-Madrid, F. (2011). The mitochondrial fission factor dynamin-related protein 1 modulates T-cell receptor signalling at the immune synapse. EMBO J, 30, 1238-50.
Benard, G., Bellance, N., James, D., Parrone, P., Fernandez, H., Letellier, T. & Rossignol, R. (2007). Mitochondrial bioenergetics and structural network organization. J Cell Sci, 120, 838-48.
Benard, G. & Rossignol, R. (2008). Ultrastructure of the mitochondrion and its bearing on function and bioenergetics. Antioxid Redox Signal, 10, 1313-42.
Buck, M. D., O'Sullivan, D. & Pearce, E. L. (2015). T cell metabolism drives immunity. J Exp Med, 212, 1345-60.
Campello, S., Lacalle, R. A., Bettella, M., Manes, S., Scorrano, L. & Viola, A. (2006). Orchestration of lymphocyte chemotaxis by mitochondrial dynamics. J Exp Med, 203, 2879-86.
Carrio, R., Bathe, O. F. & Malek, T. R. (2004). Initial antigen encounter programs CD8+ T cells competent to develop into memory cells that are activated in an antigen-free, IL-7- and IL-15-rich environment. J Immunol, 172, 7315-23.
Cassidy-Stone, A., Chipuk, J. E., Ingerman, E., Song, C., Yoo, C., Kuwana, T., Kurth, M. J., Shaw, J. T., Hinshaw, J. E., Green, D. R. & Nunnari, J. (2008). Chemical inhibition of the mitochondrial division dynamin reveals its role in Bax/Bak-dependent mitochondrial outer membrane permeabilization. Dev Cell, 14, 193-204.
Cereghetti, G. M., Stangherlin, A., Martins de Brito, O., Chang, C. R., Blackstone, C., Bernardi, P. & Scorrano, L. (2008). Dephosphorylation by calcineurin regulates translocation of Drp1 to mitochondria. Proc Natl Acad Sci USA, 105, 15803-8.
Chan, D. C. (2012). Fusion and fission: interlinked processes critical for mitochondrial health. Annu Rev Genet, 46, 265-87.
Chang, C. H., Curtis, J. D., Maggi, L. B., Jr., Faubert, B., Villarino, A. V., O'Sullivan, D., Huang, S. C., van der Windt, G. J., Blagih, J., Qiu, J., Weber, J. D., Pearce, E. J., Jones, R. G. & Pearce, E. L. (2013). Posttranscriptional control of T cell effector function by aerobic glycolysis. Cell, 153, 1239-51.
Chang, C. H., Qiu, J., O'Sullivan, D., Buck, M. D., Noguchi, T., Curtis, J. D., Chen, Q., Gindin, M., Gubin, M. M., van der Windt, G. J., Tonc, E., Schreiber, R. D., Pearce, E. J. & Pearce, E. L. (2015). Metabolic Competition in the Tumor Microenvironment Is a Driver of Cancer Progression. Cell, 162, 1229-41.
Chen, H., Detmer, S. A., Ewald, A. J., Griffin, E. E., Fraser, S. E. & Chan, D. C. (2003). Mitofusins Mfn1 and Mfn2 coordinately regulate mitochondrial fusion and are essential for embryonic development. J Cell Biol, 160, 189-200.
Chen, H., McCaffery, J. M. & Chan, D. C. (2007). Mitochondrial fusion protects against neurodegeneration in the cerebellum. Cell, 130, 548-62.
Choi, S. W., Gerencser, A. A. & Nicholls, D. G. (2009). Bioenergetic analysis of isolated cerebrocortical nerve terminals on a microgram scale: spare respiratory capacity and stochastic mitochondrial failure. J Neurochem, 109, 1179-91.
Chouchani, E. T., Pell, V. R., Gaude, E., Aksentijevic, D., Sundier, S. Y., Robb, E. L., Logan, A., Nadtochiy, S. M., Ord, E. N., Smith, A. C., Eyassu, F., Shirley, R., Hu, C. H., Dare, A. J., James, A. M., Rogatti, S., Hartley, R. C., Eaton, S., Costa, A. S., Brookes, P. S., Davidson, S. M., Duchen, M. R., Saeb-Parsy, K., Shattock, M. J., Robinson, A. J., Work, L. M., Frezza, C., Krieg, T. & Murphy, M. P. (2014). Ischaemic accumulation of succinate controls reperfusion injury through mitochondrial ROS. Nature, 515, 431-5.
Cipolat, S., Martins de Brito, O., Dal Zilio, B. & Scorrano, L. (2004). OPA1 requires mitofusin 1 to promote mitochondrial fusion. Proc Natl Acad Sci USA, 101, 15927-32.
Civiletto, G., Varanita, T., Cerutti, R., Gorletta, T., Barbaro, S., Marchet, S., Lamperti, C., Viscomi, C., Scorrano, L. & Zeviani, M. (2015). Opa1 overexpression ameliorates the phenotype of two mitochondrial disease mouse models. Cell Metab, 21, 845-54.
Cogliati, S., Frezza, C., Soriano, M. E., Varanita, T., Quintana-Cabrera, R., Corrado, M., Cipolat, S., Costa, V., Casarin, A., Gomes, L. C., Perales-Clemente, E., Salviati, L., Fernandez-Silva, P., Enriquez, J. A. & Scorrano, L. (2013). Mitochondrial cristae shape determines respiratory chain supercomplexes assembly and respiratory efficiency. Cell, 155, 16071.
Cui, G., Staron, M. M., Gray, S. M., Ho, P. C., Amezquita, R. A., Wu, J. & Kaech, S. M. (2015). IL-7-Induced Glycerol Transport and TAG Synthesis Promotes Memory CD8+ T Cell Longevity. Cell, 161, 750-61.
Dawson, A. G. (1979). Oxidation of cytosolic NADH formed during aerobic metabolism in mammalian cells. Trends in Biochemical Sciences, 4, 171-176.
de Brito, O. M. & Scorrano, L. (2008). Mitofusin 2 tethers endoplasmic reticulum to mitochondria. Nature, 456, 605-10.
Deberardinis, R. J., Lum, J. J. & Thompson, C. B. (2006). Phosphatidylinositol 3-kinase-dependent modulation of carnitine palmitoyltransferase 1A expression regulates lipid metabolism during hematopoietic cell growth. J Biol Chem, 281, 37372-80.
Everts, B., Amiel, E., Huang, S. C., Smith, A. M., Chang, C. H., Lam, W. Y., Redmann, V., Freitas, T. C., Blagih, J., van der Windt, G. J., Artyomov, M. N., Jones, R. G., Pearce, E. L. & Pearce, E. J. (2014). TLR-driven early glycolytic reprogramming via the kinases TBK1-IKKvarepsilon supports the anabolic demands of dendritic cell activation. Nat Immunol, 15, 323-32.
Ferrick, D. A., Neilson, A. & Beeson, C. (2008). Advances in measuring cellular bioenergetics using extracellular flux. Drug Discov Today, 13, 268-74.
Fox, C. J., Hammerman, P. S. & Thompson, C. B. (2005). Fuel feeds function: energy metabolism and the T-cell response. Nat Rev Immunol, 5, 844-52.
Frank, M., Duvezin-Caubet, S., Koob, S., Occhipinti, A., Jagasia, R., Petcherski, A., Ruonala, M. O., Priault, M., Salin, B. & Reichert, A. S. (2012). Mitophagy is triggered by mild oxidative stress in a mitochondrial fission dependent manner. Biochim Biophys Acta, 1823, 2297-310.
Frezza, C., Cipolat, S., Martins de Brito, O., Micaroni, M., Beznoussenko, G. V., Rudka, T., Bartoli, D., Polishuck, R. S., Danial, N. N., De Strooper, B. & Scorrano, L. (2006). OPA1 controls apoptotic cristae remodeling independently from mitochondrial fusion. Cell, 126, 177-89.
Friedman, J. R. & Nunnari, J. (2014). Mitochondrial form and function. Nature, 505, 335-43.
Gomes, L. C., Di Benedetto, G. & Scorrano, L. (2011). During autophagy mitochondria elongate are spared from degradation and sustain cell viability. Nat Cell Biol, 13, 589-98.
Houtkooper, R. H., Pirinen, E. & Auwerx, J. (2012). Sirtuins as regulators of metabolism and healthspan. Nat Rev Mol Cell Biol, 13, 225-38.
Huang, S. C., Everts, B., Ivanova, Y., O'Sullivan, D., Nascimento, M., Smith, A. M., Beatty, W., Love-Gregory, L., Lam, W. Y., O'Neill, C. M., Yan, C., Du, H., Abumrad, N. A., Urban, J. F., Jr., Artyomov, M. N., Pearce, E. L. & Pearce, E. J. (2014). Cell-intrinsic lysosomal lipolysis is essential for alternative activation of macrophages. Nat Immunol, 15, 846-55.
Ingerman, E., Perkins, E. M., Marino, M., Mears, J. A., McCaffery, J. M., Hinshaw, J. E. & Nunnari, J. (2005). Dnm1 forms spirals that are structurally tailored to fit mitochondria. J Cell Biol, 170, 1021-7.
Ishihara, N., Otera, H., Oka, T. & Mihara, K. (2013). Regulation and physiologic functions of GTPases in mitochondrial fusion and fission in mammals. Antioxid Redox Signal, 19, 389-99.
Kaech, S. M., Tan, J. T., Wherry, E. J., Konieczny, B. T., Surh, C. D. & Ahmed, R. (2003). Selective expression of the interleukin 7 receptor identifies effector CD8 T cells that give rise to long-lived memory cells. Nat Immunol, 4, 1191-8.
Krawczyk, C. M., Holowka, T., Sun, J., Blagih, J., Amiel, E., DeBerardinis, R. J., Cross, J. R., Jung, E., Thompson, C. B., Jones, R. G. & Pearce, E. J. (2010). Toll-like receptor-induced changes in glycolytic metabolism regulate dendritic cell activation. Blood, 115, 4742-9.
Labrousse, A. M., Zappaterra, M. D., Rube, D. A. & van der Bliek, A. M. (1999). C. elegans dynamin-related protein DRP-1 controls severing of the mitochondrial outer membrane. Mol Cell, 4, 815-26.
Liesa, M. & Shirihai, O. S. (2013). Mitochondrial dynamics in the regulation of nutrient utilization and energy expenditure. Cell Metab, 17, 491-506.
MacIver, N. J., Michalek, R. D. & Rathmell, J. C. (2013). Metabolic regulation of T lymphocytes. Annu Rev Immunol, 31, 259-83.
Marsboom, G., Toth, P. T., Ryan, J. J., Hong, Z., Wu, X., Fang, Y. H., Thenappan, T., Piao, L., Zhang, H. J., Pogoriler, J., Chen, Y., Morrow, E., Weir, E. K., Rehman, J. & Archer, S. L. (2012). Dynamin-related protein 1-mediated mitochondrial mitotic fission permits hyperproliferation of vascular smooth muscle cells and offers a novel therapeutic target in pulmonary hypertension. Circ Res, 110, 1484-97.
Maus, M. V., Fraietta, J. A., Levine, B. L., Kalos, M., Zhao, Y. & June, C. H. (2014). Adoptive immunotherapy for cancer or viruses. Annu Rev Immunol, 32, 189-225.
Mishra, P., Carelli, V., Manfredi, G. & Chan, D. C. (2014). Proteolytic cleavage of Opa1 stimulates mitochondrial inner membrane fusion and couples fusion to oxidative phosphorylation. Cell Metab, 19, 630-41.
Mishra, P. & Chan, D. C. (2016). Metabolic regulation of mitochondrial dynamics. J Cell Biol, 212, 379-87.
Nicholls, D. G. (2009). Spare respiratory capacity, oxidative stress and excitotoxicity. Biochem Soc Trans, 37, 1385-8.
Nicholls, D. G., Darley-Usmar, V. M., Wu, M., Jensen, P. B., Rogers, G. W. & Ferrick, D. A. (2010). Bioenergetic profile experiment using C2C12 myoblast cells. J Vis Exp.
Nunnari, J. & Suomalainen, A. (2012). Mitochondria: in sickness and in health. Cell, 148, 114559.
O'Sullivan, D. & Pearce, E. L. (2015). Targeting T cell metabolism for therapy. Trends Immunol, 36, 71-80.
O'Sullivan, D., van der Windt, G. J., Huang, S. C., Curtis, J. D., Chang, C. H., Buck, M. D., Qiu, J., Smith, A. M., Lam, W. Y., DiPlato, L. M., Hsu, F. F., Birnbaum, M. J., Pearce, E. J. & Pearce, E. L. (2014). Memory CD8(+) T cells use cell-intrinsic lipolysis to support the metabolic programming necessary for development. Immunity, 41, 75-88.
Patten, D. A., Wong, J., Khacho, M., Soubannier, V., Mailloux, R. J., Pilon-Larose, K., MacLaurin, J. G., Park, D. S., McBride, H. M., Trinkle-Mulcahy, L., Harper, M. E., Germain, M. & Slack, R. S. (2014). OPA1-dependent cristae modulation is essential for cellular adaptation to metabolic demand. EMBO J, 33, 2676-91.
Pearce, E. L. & Pearce, E. J. (2013). Metabolic pathways in immune cell activation and quiescence. Immunity, 38, 633-43.
Pearce, E. L., Poffenberger, M. C., Chang, C. H. & Jones, R. G. (2013). Fueling immunity: insights into metabolism and lymphocyte function. Science, 342, 1242454.
Pearce, E. L., Walsh, M. C., Cejas, P. J., Harms, G. M., Shen, H., Wang, L. S., Jones, R. G. & Choi, Y. (2009). Enhancing CD8 T-cell memory by modulating fatty acid metabolism. Nature, 460, 103-7.
Rambold, A. S., Cohen, S. & Lippincott-Schwartz, J. (2015). Fatty acid trafficking in starved cells: regulation by lipid droplet lipolysis, autophagy, and mitochondrial fusion dynamics. Dev Cell, 32, 678-92.
Rambold, A. S., Kostelecky, B., Elia, N. & Lippincott-Schwartz, J. (2011a). Tubular network formation protects mitochondria from autophagosomal degradation during nutrient starvation. Proc Natl Acad Sci USA, 108, 10190-5.
Rambold, A. S., Kostelecky, B. & Lippincott-Schwartz, J. (2011b). Fuse or die: Shaping mitochondrial fate during starvation. Commun Integr Biol, 4, 752-4.
Restifo, N. P., Dudley, M. E. & Rosenberg, S. A. (2012). Adoptive immunotherapy for cancer: harnessing the T cell response. Nat Rev Immunol, 12, 269-81.
Roth, D., Krammer, P. H. & Gulow, K. (2014). Dynamin related protein 1-dependent mitochondrial fission regulates oxidative signalling in T cells. FEBS Lett, 588, 1749-54.
Samant, S. A., Zhang, H. J., Hong, Z., Pillai, V. B., Sundaresan, N. R., Wolfgeher, D., Archer, S. L., Chan, D. C. & Gupta, M. P. (2014). SIRT3 deacetylates and activates OPA1 to regulate mitochondrial dynamics during stress. Mol Cell Biol, 34, 807-19.
Santel, A., Frank, S., Gaume, B., Herrler, M., Youle, R. J. & Fuller, M. T. (2003). Mitofusin-1 protein is a generally expressed mediator of mitochondrial fusion in mammalian cells. J Cell Sci, 116, 2763-74.
Schluns, K. S., Williams, K., Ma, A., Zheng, X. X. & Lefrancois, L. (2002). Cutting edge: requirement for IL-15 in the generation of primary and memory antigen-specific CD8 T cells. J Immunol, 168, 4827-31.
Sebastian, D., Hernandez-Alvarez, M. I., Segales, J., Sorianello, E., Munoz, J. P., Sala, D., Waget, A., Liesa, M., Paz, J. C., Gopalacharyulu, P., Oresic, M., Pich, S., Burcelin, R., Palacin, M. & Zorzano, A. (2012). Mitofusin 2 (Mfn2) links mitochondrial and endoplasmic reticulum function with insulin signaling and is essential for normal glucose homeostasis. Proc Natl Acad Sci USA, 109, 5523-8.
Sena, L. A., Li, S., Jairaman, A., Prakriya, M., Ezponda, T., Hildeman, D. A., Wang, C. R., Schumacker, P. T., Licht, J. D., Perlman, H., Bryce, P. J. & Chandel, N. S. (2013). Mitochondria are required for antigen-specific T cell activation through reactive oxygen species signaling. Immunity, 38, 225-36.
Serasinghe, M. N., Wieder, S. Y., Renault, T. T., Elkholi, R., Asciolla, J. J., Yao, J. L., Jabado, O., Hoehn, K., Kageyama, Y., Sesaki, H. & Chipuk, J. E. (2015). Mitochondrial division is requisite to RAS-induced transformation and targeted by oncogenic MAPK pathway inhibitors. Mol Cell, 57, 521-36.
Smith-Garvin, J. E., Koretzky, G. A. & Jordan, M. S. (2009). T cell activation. Annu Rev Immunol, 27, 591-619.
Taguchi, N., Ishihara, N., Jofuku, A., Oka, T. & Mihara, K. (2007). Mitotic phosphorylation of dynamin-related GTPase Drp1 participates in mitochondrial fission. J Biol Chem, 282, 11521-9.
van der Windt, G. J., Everts, B., Chang, C. H., Curtis, J. D., Freitas, T. C., Amiel, E., Pearce, E. J. & Pearce, E. L. (2012). Mitochondrial respiratory capacity is a critical regulator of CD8+ T cell memory development. Immunity, 36, 68-78.
van der Windt, G. J., O'Sullivan, D., Everts, B., Huang, S. C., Buck, M. D., Curtis, J. D., Chang, C. H., Smith, A. M., Ai, T., Faubert, B., Jones, R. G., Pearce, E. J. & Pearce, E. L. (2013). CD8 memory T cells have a bioenergetic advantage that underlies their rapid recall ability. Proc Natl Acad Sci USA, 110, 14336-41.
Vander Heiden, M. G., Cantley, L. C. & Thompson, C. B. (2009). Understanding the Warburg effect: the metabolic requirements of cell proliferation. Science, 324, 1029-33.
Varanita, T., Soriano, M. E., Romanello, V., Zaglia, T., Quintana-Cabrera, R., Semenzato, M., Menabo, R., Costa, V., Civiletto, G., Pesce, P., Viscomi, C., Zeviani, M., Di Lisa, F., Mongillo, M., Sandri, M. & Scorrano, L. (2015). The OPA1-dependent mitochondrial cristae remodeling pathway controls atrophic, apoptotic, and ischemic tissue damage. Cell Metab, 21, 834-44.
Wai, T. & Langer, T. (2016). Mitochondrial Dynamics and Metabolic Regulation. Trends Endocrinol Metab, 27, 105-17.
Wakabayashi, J., Zhang, Z., Wakabayashi, N., Tamura, Y., Fukaya, M., Kensler, T. W., Iijima, M. & Sesaki, H. (2009). The dynamin-related GTPase Drp1 is required for embryonic and brain development in mice. J Cell Biol, 186, 805-16.
Wang, D., Wang, J., Bonamy, G. M., Meeusen, S., Brusch, R. G., Turk, C., Yang, P. & Schultz, P. G. (2012). A small molecule promotes mitochondrial fusion in mammalian cells. Angew Chem Int Ed Engl, 51, 9302-5.
Wang, L., Ishihara, T., lbayashi, Y., Tatsushima, K., Setoyama, D., Hanada, Y., Takeichi, Y., Sakamoto, S., Yokota, S., Mihara, K., Kang, D., Ishihara, N., Takayanagi, R. & Nomura, M. (2015). Disruption of mitochondrial fission in the liver protects mice from diet-induced obesity and metabolic deterioration. Diabetologia, 58, 2371-80.
Wang, R. & Green, D. R. (2012). Metabolic checkpoints in activated T cells. Nat Immunol, 13, 907-15.
Williams, M. A. & Bevan, M. J. (2007). Effector and memory CTL differentiation. Annu Rev Immunol, 25, 171-92.
Yadava, N. & Nicholls, D. G. (2007). Spare respiratory capacity rather than oxidative stress regulates glutamate excitotoxicity after partial respiratory inhibition of mitochondrial complex I with rotenone. J Neurosci, 27, 7310-7.
Youle, R. J. & Karbowski, M. (2005). Mitochondrial fission in apoptosis. Nat Rev Mol Cell Biol, 6, 657-63.
Youle, R. J. & van der Bliek, A. M. (2012). Mitochondrial fission, fusion, and stress. Science, 337, 1062-5.
Yu, T., Robotham, J. L. & Yoon, Y. (2006). Increased production of reactive oxygen species in hyperglycemic conditions requires dynamic change of mitochondrial morphology. Proc Natl Acad Sci USA, 103, 2653-8.
Zanna C, Ghelli A, Porcelli A M, Karbowski M, Youle R J, Schimpf S, Wissinger B, Pinti M, Cossarizza A, Vidoni S, Valentino M L, Rugolo M, Carelli V. OPA1 mutations associated with dominant atrophy impair oxidative phosphorylation and mitochondrial fusion. (2008). Brain, 131, 352-67.
Zhang, Z., Wakabayashi, N., Wakabayashi, J., Tamura, Y., Song, W. J., Sereda, S., Clerc, P., Polster, B. M., Aja, S. M., Pletnikov, M. V., Kensler, T. W., Shirihai, O. S., Iijima, M., Hussain, M. A. & Sesaki, H. (2011). The dynamin-related GTPase Opa1 is required for glucose-stimulated ATP production in pancreatic beta cells. Mol Biol Cell, 22, 2235-45.
Zorzano, A., Liesa, M., Sebastian, D., Segales, J. & Palacin, M. (2010). Mitochondrial fusion proteins: dual regulators of morphology and metabolism. Semin Cell Dev Biol, 21, 56674.

What is claimed is:

1. A method of reducing immune cell activation in a subject, the method comprising administering to the subject a composition comprising one or more compounds to inhibit mitochondrial fission, or one or more compounds that promote mitochondrial fusion, or a combination thereof.

2. A method of claim 1, wherein the one or more compounds inhibiting mitochondrial fission are Drp1 inhibitors.

3. The method of claim 2, wherein the Drp1 inhibitor is Mdivi-1.

4. The method of claim 1, wherein one of the compounds that promotes mitochondrial fusion is M1.

5. The method of claim 1, wherein the composition comprises of at least one compound that inhibits mitochondrial fission and at least one compound that promotes mitochondrial fusion.

6. The method of claim 1, wherein the immune cells are T cells, macrophages, or dendritic cells.

7. A method of reducing inflammation in a subject, the method comprising administering to the subject a composition comprising one or more compounds to inhibit mitochondrial fission or one or more compounds that promote mitochondrial fusion, or a combination thereof.

8. A method of claim 7, wherein the one or more compounds inhibiting mitochondrial fission are Drp1 inhibitors.

9. The method of claim 7, wherein one of the compounds inhibiting mitochondrial fission is Mdivi-1.

10. The method of claim 7, wherein one of the compounds that promotes mitochondrial fusion is M1.

11. The method of claim 7, wherein the composition comprises at least one compound that inhibits mitochondrial fission and at least one compound that promotes mitochondrial fusion.

12. The method of claim 7, wherein the inflammation is due to induction of aerobic glycolysis.

13. The method of claim 7, wherein the inflammation is due to sepsis, obesity, rheumatoid arthritis, multiple sclerosis, Crohn's disease, irritable bowel disease, colitis, psoriasis, inflammatory liver disease, or nonalcoholic fatty liver disease.

14. The method of claim 7, wherein the inflammation is due to increase in the number of inflammatory immune cells and decrease in number of T regulatory cells.

15. The method of claim 14, further comprising the increase in number T regulatory cells.

16. The method of claim 14, further comprising the decrease in number of T helper 17 cells.

17. A method of reducing at least one detectable marker for inflammation in a subject, the method comprising administering to the subject a composition comprising one or more compounds to inhibit mitochondrial fission, or one or more compounds that promote mitochondrial fusion, or a combination thereof.

18. The method of claim 17, wherein the marker for inflammation is selected from the group consisting of cytokines, inflammatory immune cells, lactate, C-reactive protein (CRP), mitochondrial structure, mitochondrial function, and metabolic function of immune cells.

19. The method of claim 17, wherein the marker for inflammation is detected in a biological sample obtained from the subject.

20. A method of claim 17, wherein the one or more compounds inhibiting mitochondrial fission are Drp1 inhibitors.


SEQUENCE LISTING (1 sequence)

SEQ ID NO: 1 - protein, 8 aa, Artificial Sequence
  SYNTHESIZED
SIINFEKL